(19) United States
(12) Patent Application Publication
(10) Pub. No.: US 2023/0150929 A1
(43) Pub. Date: May 18, 2023

(54) SELECTIVE NON-CYCLIC NUCLEOTIDE ACTIVATORS FOR THE CAMP SENSOR EPAC1

(71) Applicant: The Board of Regents of The University of Texas System, Austin, TX (US)

(72) Inventors: Jia Zhou, Galveston, TX (US); Stephen Yarwood, Edinburgh (GB); Pingyuan Wang, Galveston, TX (US); Urszula Luchowska-Stanska, Edinburgh (GB); Boy van Basten, Edinburgh (GB)

(21) Appl. No.: 17/912,485

(22) PCT Filed: Mar. 17, 2021
(86) PCT No.: PCT/US2021/022839

§ 371 Date: Sep. 16, 2022

Related U.S. Application Data
(60) Provisional application No. 62/991,068, filed on Mar. 17, 2020.

(51) Int. Cl. C07C 311/51; C07D 215/20; C07D 215/22; C07D 239/78; C07D 217/04; C07D 487/04; C07D 213/74; C07D 209/04; C07D 231/56; C07D 213/61; C07D 231/12; C07D 307/38

(52) U.S. Cl. CPC C07C 311/51; C07D 209/04; C07D 213/61; C07D 213/74; C07D 215/20; C07D 215/22; C07D 217/04; C07D 231/12; C07D 231/56; C07D 239/78; C07D 307/38; C07D 487/04; C07C 2602/10

(57) ABSTRACT

The invention relates generally to novel EPAC1 activators, such as Formula I and II and the preparation thereof as well as the use of EPAC1 activators disclosed herein as to selectively activate EPAC1 in cells.

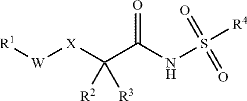

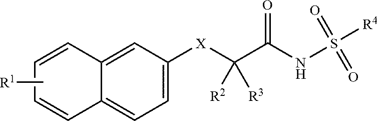

### CROSS-REFERENCE TO RELATED APPLICATIONS

This application claims the benefit of Provisional Appl. No. 62/991,068, filed Mar. 17, 2020. The content of the aforesaid applications are relied upon and are incorporated by reference herein in their entirety.

### FIELD OF THE INVENTION

The field of the invention relates generally to novel non-cyclic nucleotide EPAC1 activators and the preparation thereof as well as the use of thereof as to selectively activate EPAC1 in cells.

### BACKGROUND

This background information is provided for the purpose of making information believed by the applicant to be of possible relevance to the present invention. No admission is necessarily intended, nor should it be construed, that any of the preceding information constitutes prior art against the present invention.

Cyclic adenosine monophosphate (cAMP, FIG. 1), a well-known prototypical second messenger, is synthesized in cells from adenosine triphosphate (ATP) by adenylate cyclases (ACs).¹ cAMP plays its functional roles mainly through activation of three downstream mediators including protein kinase A (PKA),²⁻⁴ cyclic nucleotide-regulated ion channels⁵ and exchange proteins directly activated by cAMP (EPACs).⁶⁻¹⁰ EPAC proteins are multi-domain proteins that act as cAMP-regulated guanine nucleotide exchange factors (GEFs) to catalyze the exchange of guanosine diphosphate (GDP) for guanosine triphosphate (GTP) for the Ras like small GTPases (Rap1 and Rap2).^{11,}¹² Two members of EPAC protein family, EPAC1 and EPAC2, have been identified. EPAC1 and EPAC2 share about 68% sequence homology in human cells.¹³ Both isoforms can be found in different concentrations in mature and developing human tissues. In mice, the expression of EPAC1 is relatively ubiquitous, while the expression of EPAC2 is largely restricted to the central nervous system (CNS), testis and adrenal glands.¹ In cells, the EPAC protein adopts an inactive conformation when the cAMP concentration is at a low level. In contrast, EPAC enzyme activity is induced following elevations in intracellular cAMP levels.¹⁴ Subsequently, active EPAC proteins serve as GEFs for Rap proteins.¹⁵ Although the delineation of the cAMP-EPAC signaling pathway is relatively new, it has been receiving more and more attention due to its extensive and attractive biological functions within the CNS and endocrine, cardiovascular and immune systems. ^{1,}^{10,}¹⁶

Accordingly, tremendous efforts have been made to identify small-molecule EPAC modulators as chemical probes and drug candidates over the past two decades.¹⁰ In recent years, progress have made in the discovery of efficient non-cyclic nucleotide small molecule EPAC antagonists with drug-like profiles as pharmacological tools and potential drug candidates, and several developed EPAC antagonists are under preclinical studies ¹⁷⁻²⁴ as potential therapeutics for cancer,²⁵ infections,^{26,27} obesity,²⁸ chronic pain²⁹ and CNS diseases.³⁰

Growing evidence demonstrates that EPAC1 protein protects the retina against ischemia/reperfusion-induced neuronal damage³¹ and promotes neuronal differentiation and neurite proliferation.³²⁻³⁴ Use of EPAC knockout mouse models also indicates that EPAC proteins have an essential role in learning, memory and social connection.³⁵ In addition, activation of EPAC 1 suppresses inflammation via promoting the expression of suppressor of cytokine signaling 3 (SOCS3) which blocks Interleukin 6 (IL6)-induced Janus kinase (JAK)/signal transducer and activator of transcription 3 (STAT3) signaling pathway in vascular endothelial cells (VECs).³⁶⁻³⁸ EPAC1 can also exert the anti-inflammatory activity by reducing the expression of inflammatory mediators including toll-like receptor 4 (TLR4), high-mobility group box 1 (HMGB1), tumor necrosis factor α (TNFα) and interleukin-1β(IL-1(β) in human retinal endothelial cells (RECs).^{39,}⁴⁰ Moreover, EPAC plays a crucial role in cardiac cell protection⁴¹ and energy balance.^{28,}⁴² Therefore, up-regulating the activity of EPAC proteins may also offer an avenue for novel therapeutics, including drug addiction,⁴³ hyperalgesia,⁴⁴ cardiac and cardiovascular diseases^{41,45} and inflammation.⁴⁶

Currently, most reported EPAC agonists are derived from cAMP (e.g. compound 2⁴⁷ and 4⁴⁸, FIG. 1).¹⁰ However, these cAMP-derived EPAC agonists suffer from off-target side effects or poor pharmacokinetic (PK) profiles, which limit the potential of these cAMP-derived EPAC agonists for further biological applications. ^{10,}⁴⁶ Furthermore, cyclic phosphates afford limited potential for further synthetic modifications, limiting their potential as drug development candidates. Hence, potent and selective non-cyclic nucleotide small-molecule EPAC agonists with drug-like properties are urgently needed. The development of non-cyclic nucleotide EPAC1 activators of the invention meets this unmet need. The inventors have surprisingly discovered a series of non-cyclic nucleotide EPAC1 ligands, including 25g (PW0381) 25q (PW0521) 25n (PW0577), 25u (PW0606), 25e (PW0624) and 25f (PW0625), which can activate EPAC1 protein in cells and exhibit excellent selectivity towards EPAC1 over related enzymes. Moreover, 25n is better tolerated than a previously identified EPAC1-selective partial agonist (1942), in terms of protein stability of EPAC1 in cells, following long-term exposure. These new EPAC1 partial agonists may therefore not only act as useful pharmacological tools for EPAC function elucidation, but also promising drug leads for the treatment of a variety of human diseases.

### BRIEF DESCRIPTION OF THE FIGURES

FIG. 1. Structures of cAMP, and representative reported EPAC agonists.

FIG. 2. Structural modifications on I942.

FIG. 3. Relative binding affinity of selected hit compounds, in comparison with hit 3, were tested using the 8-NBD-cAMP competition assay. (A) Representative dose-response curves for EPAC1-CNBD binding ffinity. (B) Representative dose-response curves for EPAC1-ΔDEP binding affinity. The data are the mean ± SEM of at least three independent experiments.

FIG. 4. Ability of compounds 25g, 25q and 25n to promote cellular EPAC1 activity in the presence of the EPAC1 agonist, compound 2.cells. U2OS cells stably transfected with EPAC1 were stimulated with the indicated compounds. Active, GTP-bound Rap1 was pulled down from cell lysates and its levels were visualized by western blotting and quantified densitometrically. Data from at least three independent experiments presented as mean ± SEM with significant increases in Rap1-GTP levels in comparison to cells treated with 2 being indicated; ^{∗} p < 0.05 and ^{∗∗} p < 0.01.

FIG. 5. PKA activation results of representative newly discovered EPAC1 agonists. U2OS cells stably transfected with EPAC1 were treated with 100 µM of test compounds, cyclic nucleotide EPAC1 agonist 2 or 10 µM of forskolin and rolipram (F/R), cyclic AMP elevating agents, as a positive control. Active, phosphorylated VASP (P-VASP) was visualized using a phospho-specific, anti-VASP antibody. None of the test compounds promoted VASP phosphorylation in cells.

FIGS. 6A-B. Identification of compounds including 25g (PW0381) 25q (PW0521) 25n (PW0577), 25u (PW0606), 25e (PW0624) and 25f (PW0625) as EPAC1 activators in an in vitro GEF screen. The figure demonstrates relative fluorescence from EPAC1 activation assays with 10 µM of all synthesized compounds. Selected compounds have been highlighted as indicated.

FIGS. 7A-B. Ability of compounds 25e, 25f, 25g, 25q 25n and 25u to promote EPAC1 and EPAC2 activity in cells. U2OS cells stably transfected with EPAC1 or EPAC2 were stimulated with the indicated compounds. Cyclic nucleotide EPAC1 and EPAC2 agonist D-007 and S-220, respectively (2 and 4, FIG. 1) as well as 3 (FIG. 1) were used as positive controls. Active, GTP-bound Rap 1 was pulled down from cell lysates and its levels were visualized by western blotting. Experiments were carried out on at least 3 separate occasions. FIG. 7A. illustrates the experimental results for compounds 25e, 25f, and 25u. FIG B. illustrates the experimental results for compounds 25g, 25q, and 25n.

FIG. 8. The immunoblots in FIG. 7 were quantified densitometrically and the data from at least three independent experiments are presented here as mean ± SEM with significant increases in Rap1-GTP levels in EPAC1-expressing cells, relative to EPAC2-expressing cells are indicated; ^{∗∗} p < 0.01.

FIG. 9. Chemical structure of 8-NBD-cAMP

FIG. 10. Ability of selected compounds to interact with EPAC1 in cells. HEK293T cells stably transfected with EPAC1 were treated with hit compounds from the screen. EPAC1 was immunoprecipitated from cell lysates using an activation-selective antibody, visualized by western blotting and its levels were quantified densitometrically. Cyclic nucleotide EPAC1 agonist 2 was used as positive control. Compounds 25g, 25q and 25n promoted significant increases in EPAC1 immunoprecipitation, which suggests that they crossed the cell membrane, interacted with EPAC1 and by changing its conformation, enabled a more effective immunoprecipitation. Data from at least three independent experiments are presented in the bar graph as mean ± SEM with significant increases in immunoprecipitated EPAC1 levels in comparison to vehicle-treated control indicated; ^{∗∗} p < 0.01, ^{∗∗∗} p < 0.001.

### SUMMARY

It is to be understood that both the foregoing general description of the invention and the following detailed description are exemplary, and thus do not restrict the scope of the invention.

Exchange protein directly activated by cAMP (EPAC) proteins play a central role in various biological functions, and activation of the EPAC1 protein has shown potential benefits for the treatment of inflammation, energy disorders, central nervous system dysfunction and other human diseases.

1942 was previously identified and characterized as a novel non-cyclic nucleotide small molecule EPAC1 partial agonist with an IC50 value of about 35 µM. Further studies indicate that 1942 promotes the activity of the EPAC1/Rap1 pathway to suppresses pro-inflammatory signalling in vascular endothelial cells. Therefore, 1942 has been considered as a suitable hit for further hit-to-lead chemical optimization through rational drug design strategies to improve its binding and activation potencies as well as drug-like properties.

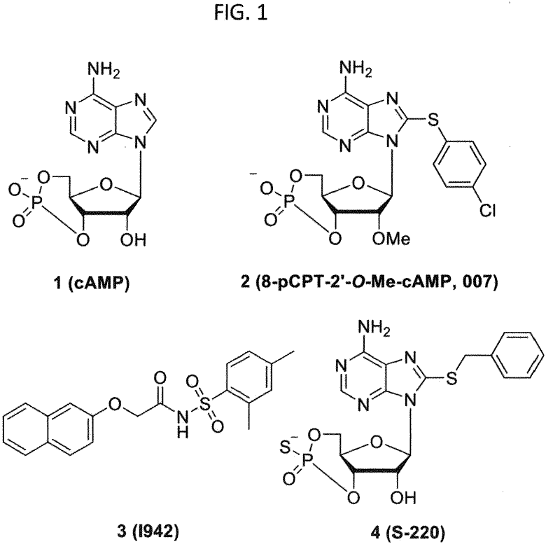

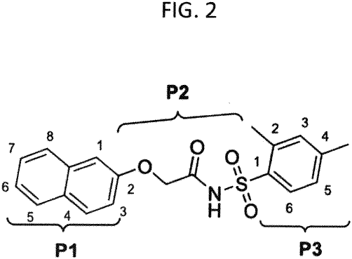

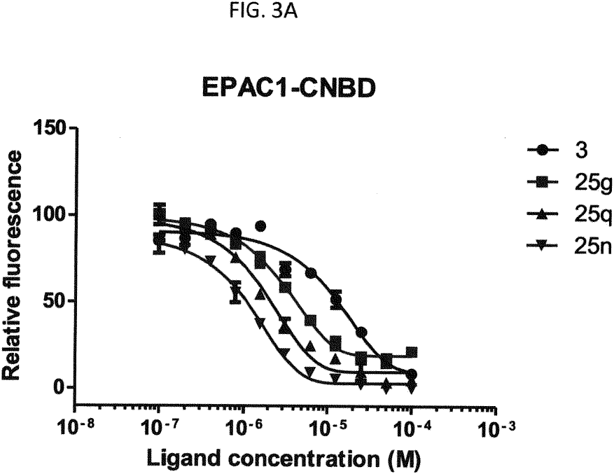

The inventors optimized the naphthyloxy group (P1, highlighted in red, FIG. 2) and the N-acylsulfonamide linker (P2, FIG. 2), as well as m-xylyl group (P3, highlighted in blue, FIG. 1) for systematic structure-activity relationship (SAR) studies, and surprisingly discovered a novel class of compounds that selectively activate EPAC1 over related enzymes.

Compounds including 25 g (PW0381), 25q (PW0521), 25n (PW0577), 25u (PW0606), 25e (PW0624), and 25f (PW0625) were identified as potent EPAC1 binders, with IC50 values ranging from low micromolar to sub-micromolar level. Additionally, the characterization in an in vitro activity assay show that these compounds are partial agonists of EPAC1. In U2OS cells the compounds induce EPAC1, but not EPAC2 or PKA activity. This is remarkable as the EPAC1 agonistic effect in vitro is only 2% of that of cAMP.

One aspect of our invention is a novel class of compounds that can activate the enzyme EPAC1 in cells and therefore form the basis of novel drugs to treat diseases, including drug addiction, hyperalgesia, cardiac and cardiovascular disease and inflammation. There is currently no IP covering this type of development.

One aspect of the invention pertains to compounds of Formula I or a pharmaceutically acceptable salt thereof, wherein:

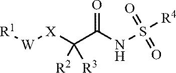

wherein:
R¹ is independently chosen from H, alkyl, alkoxy, halogen, cyan, amino, hydroxyl, NO₂, —CF₃, ,—CBr₃, —CI₃, —OCF₃,—OCBr₃, and —OCI₃;
W is independently chosen from forming a 5-12 membered aryl, heteroaryl or heterocycle having 1-3 heteroatoms
X is independently chosen from O, S, NH and CH₂;
or W and X are optionally joined to form a 5-12 membered heteroaryl or heterocycle having 1-3 heteroatoms and optionally substituted with one or more substituents selected from H, alkyl, alkoxy, halogen, cyan, amino, NO₂, hydroxyl, CF₃ or —OCF₃;
R² and R³ is independently chosen from H, alkyl and F;
R⁴ is

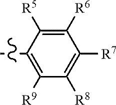

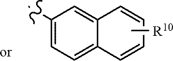

wherein R⁵, R⁶, R⁷, R⁸, R⁹ and R¹⁰ is independently chosen from H, alkyl, cycloalkyl, alkenyl, aryl, heteroaryl, benzyl, alkoxy, halogen, cyan, nitro, amino, hydroxyl, CF₃ or —OCF₃, wherein R⁵, R⁶, R⁷, R⁸, R⁹ and R¹⁰ is optionally substituted with one or more chosen substituents chosen from hydroxyl, cyan, amino, halogen, heteroaryl and heterocycle, wherein said heteroaryl and said heterocycle is optionally substituted with one or more substituents selected from H, alkyl, alkoxy, halogen, cyan, amino, NO₂, hydroxyl, CF₃ and —OCF₃;

Another aspect of the invention pertains to compounds of Formula II or a pharmaceutically acceptable salt thereof, wherein:

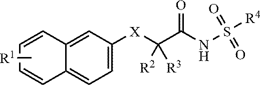

wherein:
R¹ is independently chosen from H, alkyl, alkoxy, halogen, cyan, amino, hydroxyl, nitro, —CF₃, ,—CBr₃, —CI₃, —OCF₃,—OCBr₃, and —OCI₃;
X is independently chosen from O, S, NH and CH₂;
R² and R³ is independently chosen from H, alkyl and F;
R⁴ is

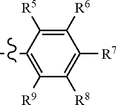

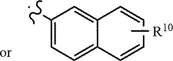

wherein R⁵, R⁶, R⁷, R⁸, R⁹ and R¹⁰ is independently chosen from H, alkyl, cycloalkyl, alkenyl, aryl, heteroaryl, benzyl, alkoxy, halogen, cyan, nitro, amino, hydroxyl, CF₃ or —OCF₃, wherein R⁵, R⁶, R⁷, R⁸, R⁹ and R¹⁰ is optionally substituted with one or more chosen substituents chosen from hydroxyl, cyan, amino, halogen heteroaryl and heterocycle, wherein said heteroaryl and said heterocycle is optionally substituted with one or more substituents selected from H, alkyl, alkoxy, halogen, cyan, amino, NO₂, hydroxyl, CF₃ and —OCF₃; Another aspect of the invention pertains to generally to use of compounds of the invention to selectively activate EPAC1 in cells.

### DETAILED DESCRIPTION

### 1.0. Definitions

For the purposes of promoting an understanding of the principles of the invention, reference will now be made to certain embodiments and specific language will be used to describe the same. It will nevertheless be understood that no limitation of the scope of the invention is thereby intended, and alterations and modifications in the illustrated article of manufacture, and further applications of the principles of the invention as illustrated therein are herein contemplated as would normally occur to one skilled in the art to which the invention relates.

Unless defined otherwise, all technical and scientific terms used herein have the same meaning as commonly understood by one of ordinary skill in the art to which this invention pertains.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

As used herein, the term “about” refers to a ±10% variation from the nominal value. It is to be understood that such a variation is always included in any given value provided herein, whether or not it is specifically referred to.

The use of “or” means “and/or” unless stated otherwise.

The use of “a” or “an” herein means “one or more” unless stated otherwise or where the use of “one or more” is clearly inappropriate.

The use of “comprise,” “comprises,” “comprising,” “include,” “includes,” and “including” are interchangeable and not intended to be limiting. Furthermore, where the description of one or more embodiments uses the term “comprising,” those skilled in the art would understand that, in some specific instances, the embodiment or embodiments can be alternatively described using the language “consisting essentially of” and/or “consisting of.”

As used herein, the terms “cell” and “cells” refer to any types of cells from any animal, such as, without limitation, rat, mice, monkey, and human.

The term “salt” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the acetate, hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These can include cations based on the alkali and alkaline earth metals, such as sodium, lithium, potassium, calcium, magnesium, and the like, as well as non-toxic ammonium, tetramethylammonium, tetraethylammonium, methylamine, dimethylamine, trimethylamine, triethylamine, ethylamine and the like (See, for example, S. M. Berge et al., “Pharmaceutical Salts,” J. Pharm. Sci., 1977, 66:1-19, which is incorporated herein by reference in its entirety).

The term “alkyl” as used herein by itself or as part of another group refers to both straight and branched chain radicals, and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The term “alkyl” may include methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, undecyl, and dodecyl.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of O, and N. The heteroatom(s) may be placed at any interior position of the heteroalkyl group or at the position at which the alkyl group is attached to the remainder of the molecule. Up to two heteroatoms may be consecutive.

The term “alkylene” as used herein refers to straight and branched chain alkyl linking groups, i.e., an alkyl group that links one group to another group in a molecule. In some embodiments, the term “alkylene” may include —(CH₂)ₙ— where n is 2-8.

The term “aryl” means a polyunsaturated hydrocarbon substituent. Aryl groups can be monocyclic or polycyclic (e.g., 2 to 3 rings that are fused together or linked covalently). Non-limiting examples of aryl and heteroaryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

The term “heteroaryl” as used herein refers to groups having 5 to 14 ring atoms; 6, 10 or 14 7π-electrons shared in a cyclic array; and containing carbon atoms and 1, 2 or 3 oxygen, nitrogen or sulfur heteroatoms. Examples of heteroaryl groups include thienyl, imadizolyl, oxadiazolyl, isoxazolyl, triazolyl, pyridyl, pyrimidinyl, pyridazinyl, furyl, pyranyl, thianthrenyl, pyrazolyl, pyrazinyl, indolizinyl, isoindolyl, isobenzofuranyl, benzoxazolyl, xanthenyl, 2H-pyrrolyl, pyrrolyl, 3H-indolyl, indolyl, indazolyl, purinyl, 4H-quinolizinyl, isoquinolyl, quinolyl, phthalazinyl, naphthyridinyl, quinazolinyl, phenanthridinyl, acridinyl, perimidinyl, phenanthrolinyl, phenazinyl, isothiazolyl, phenothiazinyl, isoxazolyl, furazanyl, and phenoxazinyl groups. Especially preferred heteroaryl groups include 1,2,3-triazole, 1,2,4-triazole, 5-amino 1,2,4-triazole, imidazole, oxazole, isoxazole, 1,2,3-oxadiazole, 1,2,4-oxadiazole, 3-amino-1,2,4-oxadiazole, 1,2,5-oxadiazole, 1,3,4-oxadiazole, pyridine, and 2-aminopyridine.

The term “heteroarylene” as used herein by itself or as part of another group refers to a heteroaryl linking group, i.e., a heteroaryl group that links one group to another group in a molecule.

An “amino” group refers to an —NH₂ group.

A “carboxylic acid” group refers to a CO₂H group.

An “alkynyl group” refers to a straight or branched chain radical of 2-20 carbon atoms, unless the chain length is limited thereto, wherein there is at least one triple bond between two of the carbon atoms in the chain, including, but not limited to, acetylene, 1-propylene, 2-propylene, and the like. In some embodiments, “alkynyl group” refers to an alkynyl chain, which is 2 to 10 carbon atoms in length. In other embodiments, “alkynyl group” refers to an alkynyl chain, which is more 2 to 8 carbon atoms in length. In further embodiments, “alkynyl group” refers to an alkynyl chain, which is from 2 to 4 carbon atoms in length.

An “amido” group refers to an —CONH₂ group. An alkylamido group refers to an -CONHR group wherein R is a straight chained, or branched alkyl. In some embodiments, R may be taken together with the —(C═O)— group to form a ring, which may be fused with, or bonded to, to a substituted or unsubstituted aryl, heteroaryl, or heterocyclic ring.

A dialkylamido group refers to an -CONRR′ group wherein R and R′ are may straight-chained, or branched, alkyl or may be taken together to form a ring, which may be fused with, or bonded to, to a substituted or unsubstituted aryl, heteroaryl, or heterocyclic ring.

The term “halogen” or “halo” or “halide” as used herein by itself or as part of another group refers to chlorine, bromine, fluorine or iodine.

The term “hydroxy” or “hydroxyl” as used herein by itself or as part of another group refers to an —OH group.

An “alkoxy” group refers to an —O—alkyl group wherein “alkyl” is as defined above. In one embodiment, the alkyl group has 1-12 carbons. In another embodiment, the alkyl group has 1-7 carbons. In a further embodiment, the alkyl group has 1-6 carbons. In another embodiment, the alkyl group has 1-4 carbons.

The term “heterocycle” or “heterocyclic ring”, as used herein except where noted, represents a stable 5- to 7-membered monocyclic-, or stable 7- to 11-membered bicyclic heterocyclic ring system, any ring of which may be saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. Rings may contain one oxygen or sulfur, one to three nitrogen atoms, or one oxygen or sulfur combined with one or two nitrogen atoms. The heterocyclic ring may be attached at any heteroatom or carbon atom that results in the creation of a stable structure.

Examples of such heterocyclic groups include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

The term “alkylamino” as used herein by itself or as part of another group refers to an amino group which is substituted with one alkyl group having from 1 to 6 carbon atoms. The term “dialkylamino” as used herein by itself or as part of another group refers to an amino group which is substituted with two alkyl groups, each having from 1 to 6 carbon atoms.

The term “arylene” as used herein by itself or as part of another group refers to an aryl linking group, i.e., an aryl group that links one group to another group in a molecule.

The term “cycloalkyl” as used herein by itself or as part of another group refers to cycloalkyl groups containing 3 to 9 carbon atoms, more preferably, 3 to 8 carbon atoms. Typical examples are cyclopropyl, cyclobutyl, cyclopentyl, cyclohexyl, cycloheptyl, cyclooctyl and cyclononyl.

Various groups are described herein as substituted or unsubstituted (i.e., optionally substituted). Optionally substituted groups may include one or more substituents independently selected from: halogen, nitro, cyano, hydroxy, amino, mercapto, formyl, carboxy, oxo, carbamoyl, alkyl, heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, and substituted or unsubstituted heteroaryl. In certain aspects, the optional substituents may be further substituted with one or more substituents independently selected from: halogen, nitro, cyano, hydroxy, amino, mercapto, formyl, carboxy, carbamoyl (—C(O)NR₂), unsubstituted alkyl, unsubstituted heteroalkyl, alkoxy, alkylthio, alkylamino, (alkyl)₂amino, alkylsulfinyl, alkyl sulfonyl, aryl sulfonyl, unsubstituted cycloalkyl, unsubstituted heterocyclyl, unsubstituted aryl, or unsubstituted heteroaryl. Exemplary optional substituents include, but are not limited to: —OH, oxo (═O), C1, —F, Br, C₁-₄alkyl, phenyl, benzyl, —NH₂, —NH(C₁₋₄alkyl), —N(C1—4alkyl)₂. —NO2, —S(C₁—₄alkyl), —SO₂(C₁—₄alkyl), —CO₂(C₁—₄alkyl), and —O(C₁—₄alkyl).

An “alkoxy” group refers to an — O—alkyl group wherein alkyl is as defined above.

A “thio” group refers to an —SH group. An “alkylthio” group refers to an -SR group wherein R is alkyl as defined above.

The term “heterocycle” or “heterocyclic ring”, as used herein except where noted, represents a stable 5- to 7-membered mono- or bicyclic or stable 7- to 10-membered bicyclic heterocyclic ring system any ring of which may be saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. Especially useful are rings containing one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of such heterocyclic groups include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

The moeity:

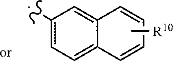

as used herein encompasses where the substituent (as exemplified by R¹⁰) is present on any secondary carbon (C) atom of the naphthyl ring system moiety.

ABBREVIATIONS USED
cAMP refers to cyclic adenosine monophosphate;
ATP refers to adenosine triphosphate;
ACs refers to adenylate cyclases;
PKA refers to protein kinase A;
EPAC refers to exchange proteins directly activated by cAMP;
GEF refers to guanine nucleotide exchange factor;
CNS refers to central nervous system;
GDP refers to guanosine diphosphate;
GTP refers to guanosine triphosphate;
TLR4 refers to toll-like receptor 4;
HMGB1 refers to high-mobility group box 1;
TNFα refers to tumor necrosis factor α;
IL-1β refers to interleukin-1β;
RECs refers to retinal endothelial cells;
SOCS3 refers to suppressor of cytokine signaling 3;
VECs refers to vascular endothelial cells;
IL6 refers to interleukin 6;
JAK refers to Janus kinase;
STAT3 refers to signal transducer and activator of transcription 3;
PK refers to pharmacokinetics;
HTS refers to high-throughput screening;
VCAM1 refers to vascular cell adhesion molecule 1;
SAR refers to structure-activity relationship;
THF refers to tetrahydrofuran;
DMF refers to N,N-dimethylformamide;
MOMC1 refers to chloromethyl methyl ether;
Boc refers to tert-butyl carbamate;
DEAD refers to diethyl azodicarboxylate;
DMAP refers to 4-dimethylaminopyridine;
EDCI, refers to 1-ethyl-(3-dimethylaminopropyl)carbonyldiimide hydrochloride;
Pd(dppf)C12 refers to 1′1-bis(diphenylphosphino)fewocene palladium (II)chloride;
XantPhos refers to 4,5-bis(diphenylphosphino)-9,9-dimethylxanthene;
RFI refers to Relative fluorescence intensity;
CNBD refers to cAMP binding domain;
GPCR refers to G protein coupled receptor;
TNFα refers to Tumor necrosis factor-alpha;
HUVECs refers to Human Umbilical Vein Endothelial Cells;
TLC refers to thin-layer chromatography;
UV refers to ultraviolet;
TMS refers to tetramethylsilane;
HRMS refers to high-resolution mass spectra;
HPLC, refers to high-performance liquid chromatography;
TFA refers to trifluoroacetic acid;
EtOAc refers to ethyl acetate; and
DCM refers to dichloromethane.

### Compounds

The inventors have surprisingly discovered certain novel small molecules that may be used as to selectively activate EPAC1 in cells.

One aspect of the invention pertains to compounds of Formula I, or a pharmaceutically acceptable salt thereof, wherein:

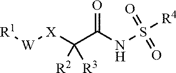

wherein:
R¹ is independently chosen from H, alkyl, alkoxy, halogen, cyan, amino, hydroxyl, NO₂, CF₃ and —OCF₃;
W is independently chosen from forming a 5-12 membered aryl, heteroaryl or heterocycle having 1-3 heteroatoms
X is independently chosen from O, S, NH and CH₂;
or W and X are optionally joined to form a 5-12 membered heteroaryl or heterocycle having 1-3 heteroatoms and optionally substituted with one or more substituents selected from H, alkyl, alkoxy, halogen, cyan, amino, NO₂, hydroxyl, CF₃ or —OCF₃;
R² and R³ is independently chosen from H, alkyl and F;
R⁴ is

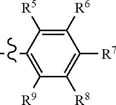

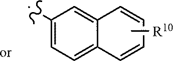

wherein R⁵, R⁶, R⁷, R⁸, R⁹ and R¹⁰ is independently chosen from H, alkyl, cycloalkyl, alkenyl, aryl, heteroaryl, benzyl, alkoxy, halogen, cyan, nitro, amino, hydroxyl, CF₃ and —OCF₃, wherein R⁵, R⁶, R⁷, R⁸, R⁹ and R¹⁰ is optionally substituted with one or more chosen substituents chosen from hydroxyl, cyan, amino, halogen, heteroaryl and heterocycle, wherein heteroaryl and heterocycle is optionally substituted with one or more substituents selected from H, alkyl, alkoxy, halogen, cyan, amino, NO₂, hydroxyl, CF₃ and —OCF₃;

In some embodiments, R⁴ is selected from the group consisting of 3,5-dimethylphenyl, 2-fluorophenyl,3-fluorophenyl, 4-fluorophenyl, 3-fluoro-4-nitrophenyl, 3-fluoro-4-aminophenyl, 2,4-dimethoxyphenyl, 2,5-dimethoxyphenyl, 3,4-dimethoxyphenyl, 3-bromo-5-methylphenyl, 3-bromo-5-methylphenyl, 3,5-dichlorophenyl, 2-methoxy-4-nitrophenyl, 2-methoxy-4-aminophenyl, 2,5-dimethxoylphenyl, 3,4-dimethoxyphenyl, 2-naphthyl, 3-(5-fluoropyridin-3-yl)-5-methylphenyl, 3-(furan-2-yl)-5-methylphenyl, 3-methyl-5-(1-methyl-1H-pyrazol-5-ylphenyl, 3-methyl5-(3-(trifluoromethyl)pyridin-2-yl)aminophenyl, 3-methyl5-(5 -(trifluoromethyl)pyridin-2-yl)aminophenyl, 4-methylphenyl, 3 -nitrophenyl, phenyl, 3-methoxyphenyl, cyclohexyl, 3-(3-furanyl)phenyl, 3-biphenyl, methyl 3-benzoyl, and 2,4-dimethylphenyl.

Another aspect of the invention pertains to compounds of Formula II, or a pharmaceutically acceptable salt thereof wherein:

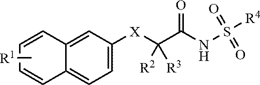

wherein:
R¹ is independently chosen from H, alkyl, alkoxy, halogen, cyan, amino, hydroxyl, nitro, CF₃ and —OCF₃;
X is independently chosen from O, S, NH and CH₂;
R² and R³ is independently chosen from H, alkyl and F;
R⁴ is [00136]

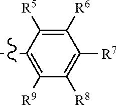

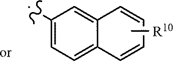

wherein R⁵, R⁶, R⁷, R⁸, R⁹ and R¹⁰ is independently chosen from H, alkyl, cycloalkyl, alkenyl, aryl, heteroaryl, benzyl, alkoxy, halogen, cyan, nitro, amino, hydroxyl, CF₃ and —OCF₃, wherein R⁵, R⁶, R⁷, R⁸, R⁹ and R¹⁰ is optionally substituted with one or more chosen substituents chosen from hydroxyl, cyan, amino, halogen heteroaryl and heterocycle, wherein said heteroaryl and said heterocycle is optionally substituted with one or more substituents selected from H, alkyl, alkoxy, halogen, cyan, amino, NO₂, hydroxyl, CF₃ and —OCF₃;

In some embodiments, R⁴ is selected from the group consisting of 3,5-dimethylphenyl, 2-fluorophenyl,3-fluorophenyl, 4-fluorophenyl, 3-fluoro-4-nitrophenyl, 3-fluoro-4-aminophenyl, 2,4-dimethoxyphenyl, 2,5-dimethoxyphenyl, 3,4-dimethoxyphenyl, 3-bromo-5-methylphenyl, 3-bromo-5-methylphenyl, 3,5-dichlorophenyl, 2-methoxy-4-nitrophenyl, 2-methoxy-4-aminophenyl, 2,5-dimethxoylphenyl, 3,4-dimethoxyphenyl, 2-naphthyl, 3-(5-fluoropyridin-3 -yl)-5-methylphenyl, 3 -(furan-2-yl)-5 -methylphenyl, 3 -methyl-5-(1-methyl-1H-pyrazol-5-ylphenyl, 3-methyl5-(3-(trifluoromethyl)pyridin-2-yl)aminophenyl, 3-methyl5-(5-(trifluoromethyl)pyridin-2-yl)aminophenyl, 4-methylphenyl, 3-nitrophenyl, phenyl, 3-methoxyphenyl, cyclohexyl, 3-(3-furanyl)phenyl, 3-biphenyl, methyl 3-benzoyl, and 2,4-dimethylphenyl.

Another aspect of the invention pertains to compounds of Formula IIa, or pharmaceutically acceptable salts thereof wherein:

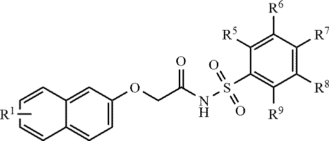

R¹ is independently chosen from H, alkyl, alkoxy, halogen, cyan, amino, hydroxyl, nitro, —CF₃, ,—CBr₃, —CI₃, —OCF₃,—OCBr₃, and —OCI₃;
wherein R⁵, R⁶, R⁷, R⁸, and R⁹ is independently chosen from H, alkyl, cycloalkyl, alkenyl, aryl, heteroaryl, benzyl, alkoxy, halogen, cyan, nitro, amino, hydroxyl, CF₃ and —OCF₃, wherein R⁵, R⁶, R⁷, R⁸, and R⁹ is optionally substituted with one or more chosen substituents chosen from hydroxyl, cyan, amino, halogen, heteroaryl and heterocycle, wherein said heteroaryl and said heterocycle is optionally substituted with one or more substituents selected from H, alkyl, alkoxy, halogen, cyan, amino, NO₂, hydroxyl, CF₃ and —OCF₃;

In further embodiment, the invention encompasses any one of the following compounds or a pharmaceutically acceptable sale thereof:

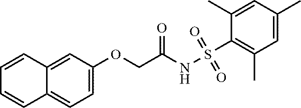 [SMILES: Cc1cc(C)c(S(=O)(=O)NC(=O)COc2ccc3ccccc3c2)c(C)c1]

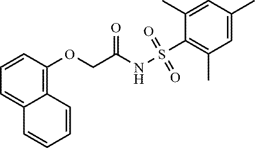 [SMILES: Cc1cc(C)c(S(=O)(=O)NC(=O)COc2cccc3ccccc23)c(C)c1]

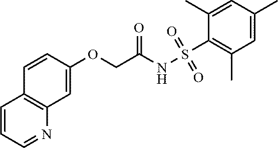 [SMILES: Cc1cc(C)c(S(=O)(=O)NC(=O)COc2ccc3cccnc3c2)c(C)c1]

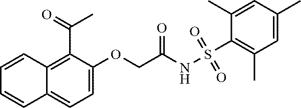 [SMILES: CC(=O)c1c(OCC(=O)NS(=O)(=O)c2c(C)cc(C)cc2C)ccc2ccccc12]

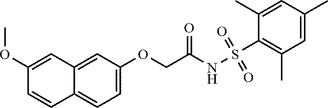 [SMILES: COc1ccc2ccc(OCC(=O)NS(=O)(=O)c3c(C)cc(C)cc3C)cc2c1]

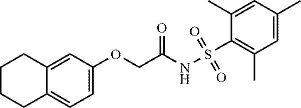 [SMILES: Cc1cc(C)c(S(=O)(=O)NC(=O)COc2ccc3c(c2)CCCC3)c(C)c1]

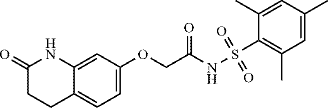 [SMILES: Cc1cc(C)c(S(=O)(=O)NC(=O)COc2ccc3c(c2)NC(=O)CC3)c(C)c1]

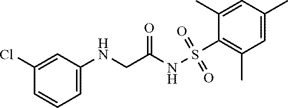 [SMILES: Cc1cc(C)c(S(=O)(=O)NC(=O)CNc2cccc(Cl)c2)c(C)c1]

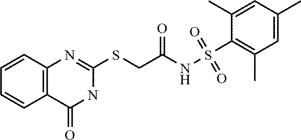 [SMILES: Cc1cc(C)c(S(=O)(=O)NC(=O)CSc2nc3ccccc3c(=O)[nH]2)c(C)c1]

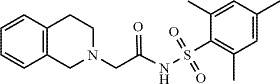 [SMILES: Cc1cc(C)c(S(=O)(=O)NC(=O)CN2CCc3ccccc3C2)c(C)c1]

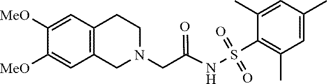 [SMILES: COc1cc2c(cc1OC)CN(CC(=O)NS(=O)(=O)c1c(C)cc(C)cc1C)CC2]

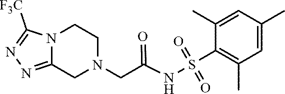 [SMILES: Cc1cc(C)c(S(=O)(=O)NC(=O)CN2CCn3c(nnc3C(F)(F)F)C2)c(C)c1]

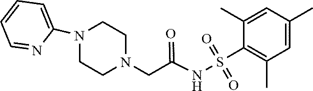 [SMILES: Cc1cc(C)c(S(=O)(=O)NC(=O)CN2CCN(c3ccccn3)CC2)c(C)c1]

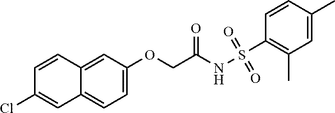 [SMILES: Cc1ccc(S(=O)(=O)NC(=O)COc2ccc3cc(Cl)ccc3c2)c(C)c1]

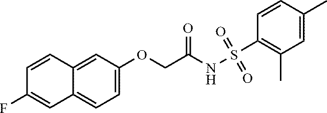 [SMILES: Cc1ccc(S(=O)(=O)NC(=O)COc2ccc3cc(F)ccc3c2)c(C)c1]

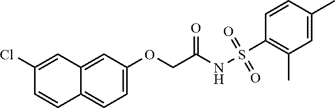 [SMILES: Cc1ccc(S(=O)(=O)NC(=O)COc2ccc3ccc(Cl)cc3c2)c(C)c1]

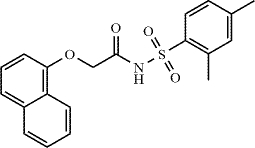 [SMILES: Cc1ccc(S(=O)(=O)NC(=O)COc2cccc3ccccc23)c(C)c1]

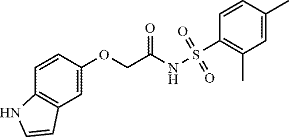 [SMILES: Cc1ccc(S(=O)(=O)NC(=O)COc2ccc3[nH]ccc3c2)c(C)c1]

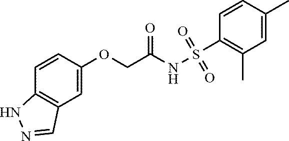 [SMILES: Cc1ccc(S(=O)(=O)NC(=O)COc2ccc3[nH]ncc3c2)c(C)c1]

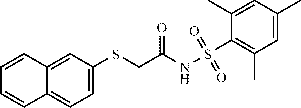 [SMILES: Cc1cc(C)c(S(=O)(=O)NC(=O)CSc2ccc3ccccc3c2)c(C)c1]

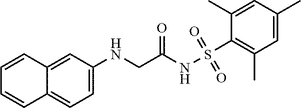 [SMILES: Cc1cc(C)c(S(=O)(=O)NC(=O)CNc2ccc3ccccc3c2)c(C)c1]

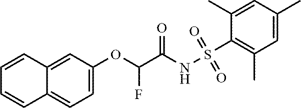 [SMILES: Cc1cc(C)c(S(=O)(=O)NC(=O)C(F)Oc2ccc3ccccc3c2)c(C)c1]

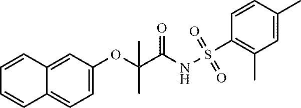 [SMILES: Cc1ccc(S(=O)(=O)NC(=O)C(C)(C)Oc2ccc3ccccc3c2)c(C)c1]

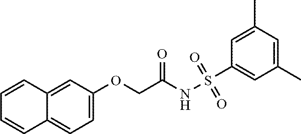 [SMILES: Cc1cc(C)cc(S(=O)(=O)NC(=O)COc2ccc3ccccc3c2)c1]

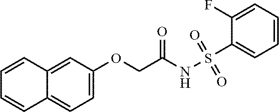 [SMILES: O=C(COc1ccc2ccccc2c1)NS(=O)(=O)c1ccccc1F]

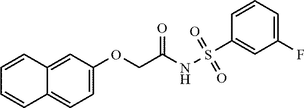 [SMILES: O=C(COc1ccc2ccccc2c1)NS(=O)(=O)c1cccc(F)c1]

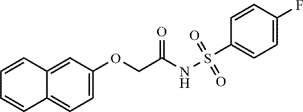 [SMILES: O=C(COc1ccc2ccccc2c1)NS(=O)(=O)c1ccc(F)cc1]

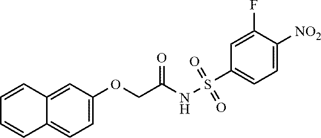 [SMILES: O=C(COc1ccc2ccccc2c1)NS(=O)(=O)c1ccc([N+](=O)[O-])c(F)c1]

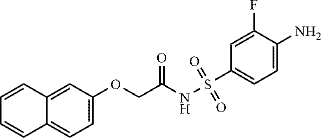 [SMILES: Nc1ccc(S(=O)(=O)NC(=O)COc2ccc3ccccc3c2)cc1F]

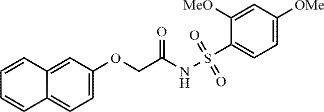 [SMILES: COc1ccc(S(=O)(=O)NC(=O)COc2ccc3ccccc3c2)c(OC)c1]

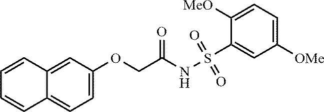 [SMILES: COc1ccc(OC)c(S(=O)(=O)NC(=O)COc2ccc3ccccc3c2)c1]

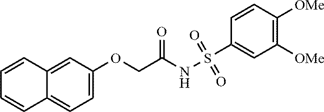 [SMILES: COc1ccc(S(=O)(=O)NC(=O)COc2ccc3ccccc3c2)cc1OC]

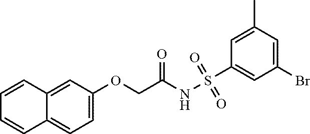 [SMILES: Cc1cc(Br)cc(S(=O)(=O)NC(=O)COc2ccc3ccccc3c2)c1]

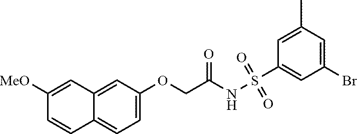 [SMILES: COc1ccc2ccc(OCC(=O)NS(=O)(=O)c3cc(C)cc(Br)c3)cc2c1]

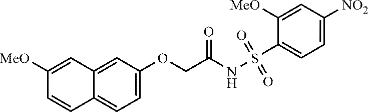 [SMILES: COc1ccc2ccc(OCC(=O)NS(=O)(=O)c3ccc([N+](=O)[O-])cc3OC)cc2c1]

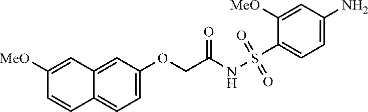 [SMILES: COc1ccc2ccc(OCC(=O)NS(=O)(=O)c3ccc(N)cc3OC)cc2c1]

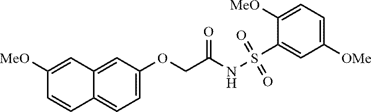 [SMILES: COc1ccc(OC)c(S(=O)(=O)NC(=O)COc2ccc3ccc(OC)cc3c2)c1]

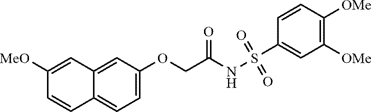 [SMILES: COc1ccc2ccc(OCC(=O)NS(=O)(=O)c3ccc(OC)c(OC)c3)cc2c1]

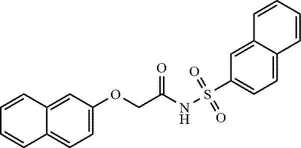 [SMILES: O=C(COc1ccc2ccccc2c1)NS(=O)(=O)c1ccc2ccccc2c1]

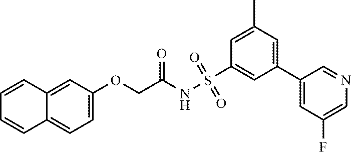 [SMILES: Cc1cc(-c2cncc(F)c2)cc(S(=O)(=O)NC(=O)COc2ccc3ccccc3c2)c1]

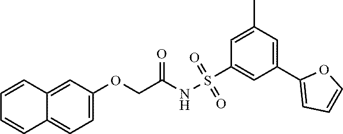 [SMILES: Cc1cc(-c2ccco2)cc(S(=O)(=O)NC(=O)COc2ccc3ccccc3c2)c1]

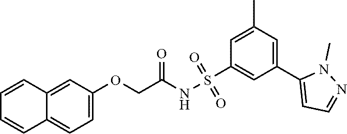 [SMILES: Cc1cc(-c2ccnn2C)cc(S(=O)(=O)NC(=O)COc2ccc3ccccc3c2)c1]

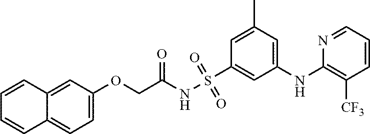 [SMILES: Cc1cc(Nc2ncccc2C(F)(F)F)cc(S(=O)(=O)NC(=O)COc2ccc3ccccc3c2)c1]

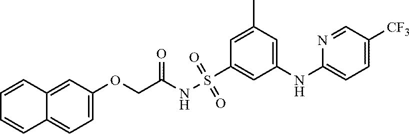 [SMILES: Cc1cc(Nc2ccc(C(F)(F)F)cn2)cc(S(=O)(=O)NC(=O)COc2ccc3ccccc3c2)c1]

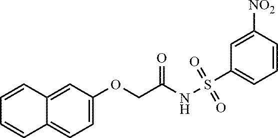 [SMILES: O=C(COc1ccc2ccccc2c1)NS(=O)(=O)c1cccc([N+](=O)[O-])c1]

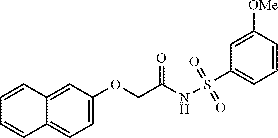 [SMILES: COc1cccc(S(=O)(=O)NC(=O)COc2ccc3ccccc3c2)c1]

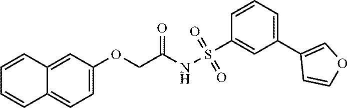 [SMILES: O=C(COc1ccc2ccccc2c1)NS(=O)(=O)c1cccc(-c2ccoc2)c1]

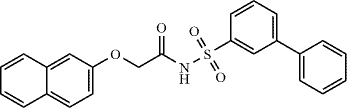 [SMILES: O=C(COc1ccc2ccccc2c1)NS(=O)(=O)c1cccc(-c2ccccc2)c1]

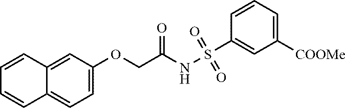 [SMILES: COC(=O)c1cccc(S(=O)(=O)NC(=O)COc2ccc3ccccc3c2)c1]

A further aspect of the invention pertains to compounds of Formula IIb, or a pharmaceutically acceptable salt thereof wherein:

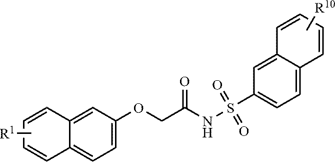

wherein:
R¹ is independently chosen from H, alkyl, alkoxy, halogen, cyan, amino, hydroxyl, nitro, —CF₃, ,—CBr₃, —CI₃, —OCF₃,—OCBr₃, and —OCI₃;
wherein R¹⁰ is independently chosen from H, alkyl, cycloalkyl, alkenyl, aryl, heteroaryl, benzyl, alkoxy, halogen, cyan, nitro, amino, hydroxyl, —CF₃, ,—CBr₃, —CI₃, —OCF₃,—OCBr₃, and —OCI₃, wherein R¹⁰ is optionally substituted with one or more chosen substituents chosen from hydroxyl, cyan, amino, halogen, heteroaryl and heterocycle, wherein said heteroaryl and said heterocycle is optionally substituted with one or more substituents selected from H, alkyl, alkoxy, halogen, cyan, amino, NO₂, hydroxyl, CF₃ and —OCF₃;

Another aspect of the invention pertains to compounds of Formula IIc, or a pharmaceutically acceptable salt thereof, wherein:

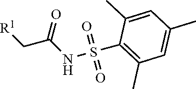

R¹ of Formula IIc may be a 5-12 membered heteroaryl or heterocycle having 1-3 heteroatoms and optionally substituted with one or more substituents selected from H, alkyl, alkoxy, halogen, cyan, amino, NO₂, hydroxyl, CF₃ and —OCF₃;
R¹ of Formula IIc may be chosen from any of the following moieties:

| | | |
|---|---|---|
| 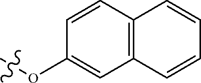 | 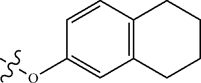 | 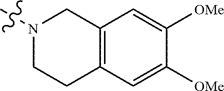 |
| 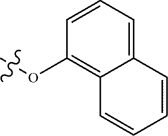 | 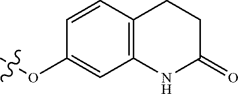 | 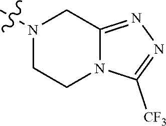 |
| 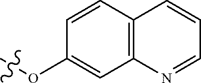 | 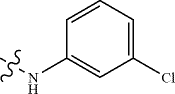 | 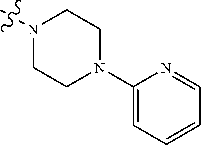 |
| 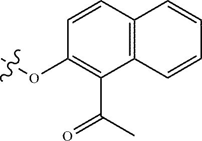 | 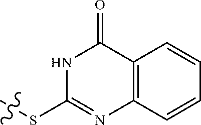 | |
| 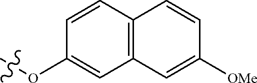 | 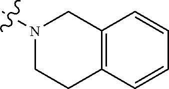 | |

Another aspect of the invention pertains to compounds of Formula IId, or a pharmaceutically acceptable salt thereof, wherein:

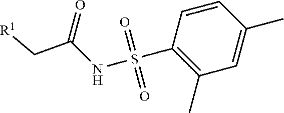

R¹ of Formula IId may be a 5-12 membered heteroaryl or heterocycle having 1-3 heteroatoms and optionally substituted with one or more substituents selected from H, alkyl, alkoxy, halogen, cyan, amino, NO₂, hydroxyl, CF₃ or —OCF₃;
R¹ of Formula IId may be chosen from any of the following moieties:

| | |
|---|---|
| 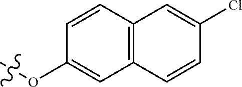 | 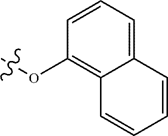 |
| 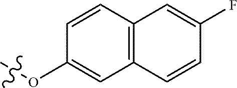 | 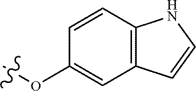 |
| 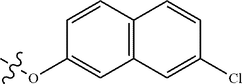 | 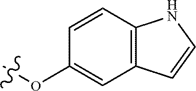 |

Another aspect of the invention pertains to compounds of Formula IIe, or pharmaceutically acceptable salts thereof wherein:

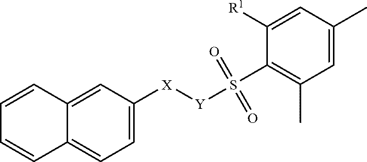

R¹ of Formula IIe may be H or alkyl (e.g., methyl);
X of Formula IIe may be O, S, or amino (such as NH);
Y of Formula IIe may be chosen from any of the following moieties:

| | |
|---|---|
| 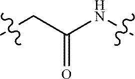 | 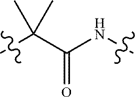 |
| 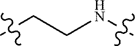 | 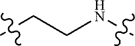 |
| 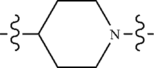 | 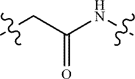 |
| 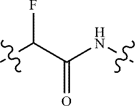 | |

In further embodiments, the inventions encompasses compounds of Formula IIe wherein:

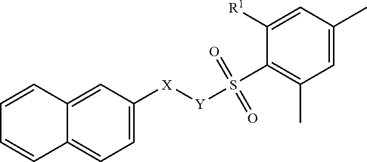

| | | |
|---|---|---|
| R¹ | X | Y |
| alkyl | O orS | 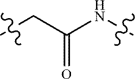 |
| alkyl | amino (e.g. NH) | 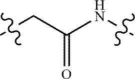 |
| alkyl | O orS | 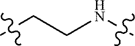 |
| alkyl | O orS | 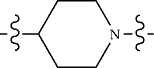 |
| alkyl | OorS | 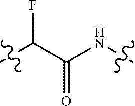 |
| H | O or S | 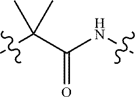 |
| H | O or S | 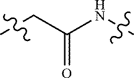 |
| H | O orS | 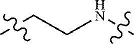 |

Another aspect of the invention pertains to compounds of Formula IIf, or a pharmaceutically acceptable sal thereof wherein:

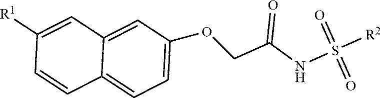

wherein R¹ is H, alkoxy (e.g. methoxy, ethoxy, n-propoxy, isopropoxy) and R² is selected from the group consisting of 3,5-dimethylphenyl, 2-fluorophenyl,3-fluorophenyl, 4-fluorophenyl, 3-fluoro-4-nitrophenyl, 3-fluoro-4-aminophenyl, 2,4-dimethoxyphenyl, 2,5-dimethoxyphenyl, 3,4-dimethoxyphenyl, 3-bromo-5-methylphenyl, 3-bromo-5-methylphenyl, 3,5-dichlorophenyl, 2-methoxy-4-nitrophenyl, 2-methoxy-4-aminophenyl, 2,5-dimethxoylphenyl, 3,4-dimethoxyphenyl, 2-naphthyl, 3-(5-fluoropyridin-3-yl)-5-methylphenyl, 3-(furan-2-yl)-5-methylphenyl, 3-methyl-5-(1-methyl-1H-pyrazol-5-ylphenyl, 3-methyl5-(3 -(trifluoromethyl)pyridin-2-yl)aminophenyl, 3 -methyl5 -(5-(trifluoromethyl)pyridin-2-yl)aminophenyl, 4-methylphenyl, 3 -nitrophenyl, phenyl, 3-methoxyphenyl, cyclohexyl, 3-(3-furanyl)phenyl, 3-biphenyl, methyl 3-benzoyl, and 2,4-dimethylphenyl.

### 2.1. Synthesis of Compounds of the Invention

The description of preparation of certain compounds of the invention is meant to be exemplary of certain embodiments of the invention. The reagents and reactant used for synthetic conversions outlined herein and below is merely exemplary. The invention contemplates using the same or different reagents discussed herein to achieve preparation of the compounds of the invention.

Certain embodiments of the invention may be synthesized using the synthetic routes for these newly synthesized EPAC1 partial agonists are outlined in Schemes 1-5. The m-xylyl group of compound 3 was replaced with 2,4,6-trimethylbenzene group to obtain the compound 9a, and its synthetic procedure is depicted in Scheme 1. The intermediate 6 was obtained by reaction of the starting material - 2,4,6-trimethylbenzenesulfonyl chloride (5), with NH_{3•}H₂O in THF. Coupling of intermediate 6 with commercially available 2-bromoacetyl bromide gave the intermediate 7. Compound 9a may be produced via substitution reaction of intermediate 7 with commercially available naphthalen-2-ol (8a) in the presence of K₂CO₃ in a yield of 68%. Compounds 9b-m may be prepared by further modifications of compound 7 through the replacement of the P1 moiety with various bicyclic or heterocyclic rings (Scheme 1). These molecules may be synthesized by reaction of intermediate 7 with commercially available materials 8b-m following a similar synthetic procedure to that of compound 9a.

Scheme 1. Synthesis of compounds 9a-m with modification on the P1 moiety^{a}

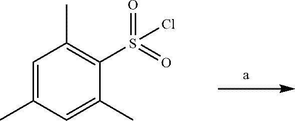

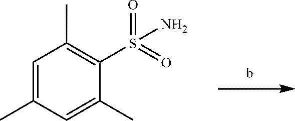

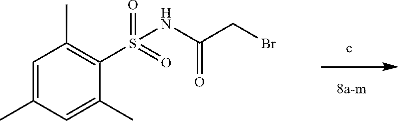

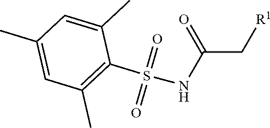

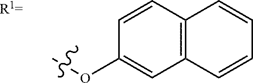

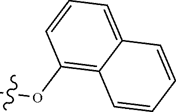

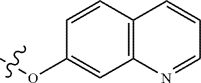

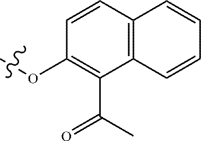

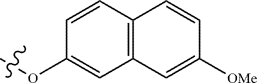

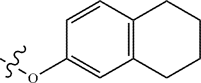

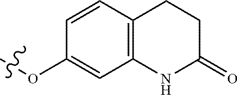

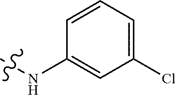

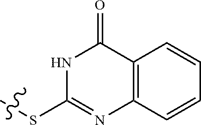

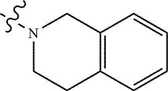

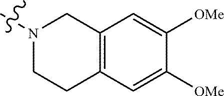

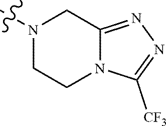

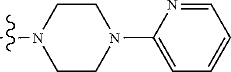

| | |
|---|---|
| 8a = naphthalen-2-ol | 8h = 3-chloroaniline |
| 8b = naphthalen-1-ol | 8i = 2-mercaptoquinazolin-4(3H)-one |
| 8c = quinolin-7-ol | 8j = 1,2,3,4-tetrahydroisoquinoline |
| 8d = 1-acetyl-2-naphthol | 8k = 6,7-dimethoxy-1,2,3,4-tetrahydroisoquinoline |
| 8e = 7-methoxynaphthalen-2-ol | 8l= 3-(trifluoromethyl)-5,6,7,8-tetrahydro- |
| 8f = 5,6,7,8-tetrahydronaphthalen-2-ol | [1,2,4]triazolo[4,3-a]pyrazine |
| 8g = 7-hydroxy-3,4-dihydroquinolin-2(1H)-one | 8m = 1-(pyridin-2-yl)piperazine |

^{a}Reagents and conditions: (a) NH₄OH(_{aq}), THF, 0° C. to rt, overnight, 94%; (b) 2-bromoacetyl bromide, toluene, reflux, 5 h, 67%; (c) 8a-m, K₂CO₃, dry DMF, rt, overnight, 48%-81%.

Halonaphthols 8n-p may be prepared from the corresponding bromonaphthols via a MOM-protection/lithiation-trapping/deprotection sequence (Scheme 2). Then, a range of aryloxyacetic acids 10n-s may be prepared by reaction of the corresponding arylalcohol 8 with ethylbromoacetate and K₂CO₃ in acetone at reflux for 16 h, followed by a solvent swap to MeOH and hydrolysis using aqueous NaOH to give 10n-s in 11-99% yields. The required sulfonylamide was synthesized by reaction of 2,4-dimethylbenzenesulfonyl chloride 11 with aqueous ammonia in THF, giving the product in 93% yield. Finally, an EDCI-mediated amide coupling produced target compounds 9n-s in 16-88% yield.

Scheme 2. Synthesis of m-xylyl compounds 9n-s with modification on the P1 moiety^{a}

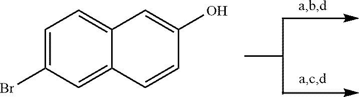

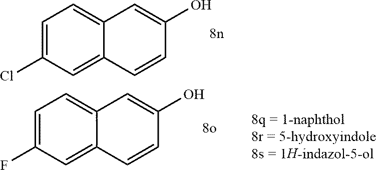

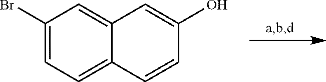

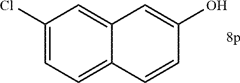

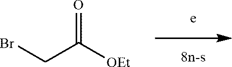

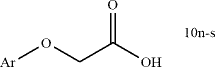

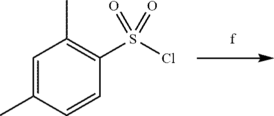

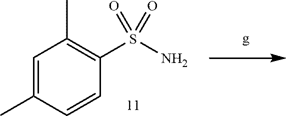

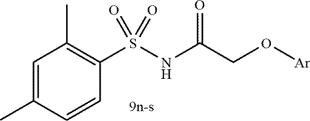

^{a}Reagents and conditions: (a) NaH, THF, rt, 0.5 h then MOMCl, THF, rt, 2 h, 58-60%; (b) n-BuLi, THF, -78° C., 0.5 h then N-chlorosuccinimide, THF, -78° C. to rt, 16 h, 21-29%; (c) n-BuLi, THF, -78° C., 0.5 h then N-fluorobenzenesulfonimide, THF, -78° C. to rt, 16 h, 38%; (d) HCl(_{aq}), MeOH, 50° C., 2 h, 86-95%; (e) 8n-s, K₂CO₃, acetone, reflux, 16 h then NaOH(_{aq}), MeOH, rt, 3 h, 11-99%; (f) NH₄OH(_{aq}), THF, rt, 16 h, 93%; (g) EDCI, DMAP, CH₂Cl₂, rt, 72 h, 16-88%.

As depicted in Scheme 3, compounds 12a-g were prepared by replacing the P2 moiety of compound 3 with different linkers to investigate the P2 role in EPAC1 binding potency. Compounds 12a and 12b were obtained following a similar synthetic procedure to that of compound 9a by substitution reaction of intermediate 7 with compounds 13 and 14, respectively. The intermediates 15 and 16 were produced via Mitsnobu coupling reaction with compound 8a as the starting reagent. Deprotection of intermediates 15 and 16 followed by coupling with 5 led to compounds 12c and 12d, respectively. Intermediate 17 was synthesized from compound 8a and ethyl 2-bromo-2-fluoroacetate with the K₂CO₃ as the base. Hydrolysis of the intermediate 17 under basic conditions yielded the key intermediate 18, followed by the subsequent coupling with intermediate 6 leading to the final compound 12e in a yield of 86%. m-Xylyl analogues 12f and 12 g were also synthesized for direct comparison to 3. Dimethylnaphthoxyacid 19 was synthesized via treatment of 2-naphthol with chloroform and acetone in the presence of sodium hydroxide, giving 19 in 22% after reflux for 4 h. A carbodiimide coupling with sulfonamide 11 then gave 12f in 8% yield. Naphthoxyamine 20 was obtained from 2-naphthol after reaction with 2-chloroethylamine in the presence of a base (KOH) in 57% yield, after which reaction with the required sulfonyl chloride gave 12 g in 36%.

Scheme 3. Synthesis of compounds 12a-g with modification on the P2 moiety^{a}

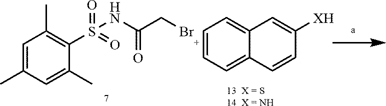

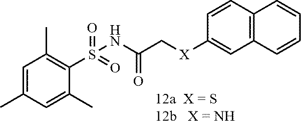

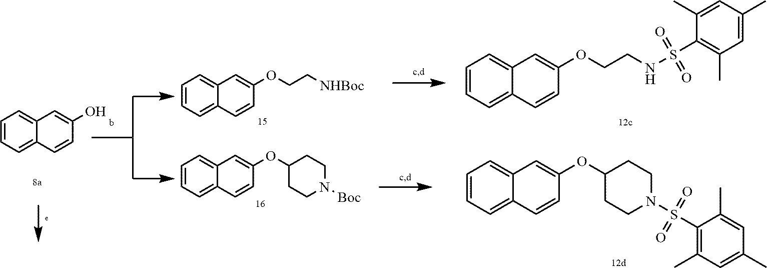

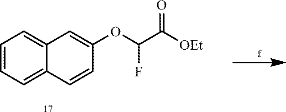

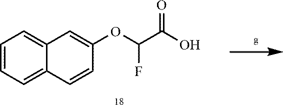

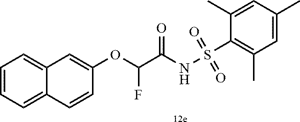

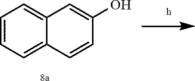

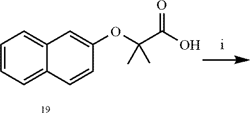

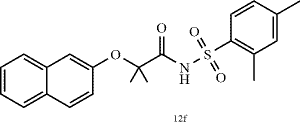

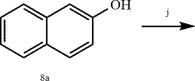

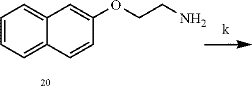

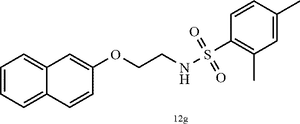

^{a}Reagents and conditions: (a) NaH, THF, 0° C. to rt, overnight, 40-76%; (b) tert-butyl (2-hydroxyethyl)carbamate or tert-butyl 4-hydroxypiperidine-1-carboxylate, PPh₃, DEAD, THF, rt, overnight, 81-88%; (c) CF₃COOH, CH₂Cl₂, rt, 5 h, quant.; (d) 5, NEt₃, DMAP, CH₂Cl₂, rt, 8 h, 91-92%; (e) ethyl 2-bromo-2-fluoroacetate, K₂CO₃, dry DMF, rt, overnight, 40%; (f) i) LiOH, THF, H₂O, rt, overnight; ii) 4N HCl, 76%; (g) 6, EDCI, DAMP, DMF, rt, overnight, 86%; (h) 2-bromo-2-methylpropanoic acid, acetone, CHC1₃, NaOH, reflux, 4 h, 22%; (i) EDCI, DMAP, CH₂Cl₂, rt, 48 h, 8%; (j) 2-chloroethylamine hydrochloride, KOH, 3:1 PhMe:dioxane, reflux, 18 h, 57%; (k) 2,4-dimethylbenzenesulfonyl chloride, CH₂Cl₂, rt, 20 min, 36%.

Compounds 21 and 22 were synthesized from corresponding compounds 8a and 8e, and were further hydrolyzed into intermediates 23 and 24, respectively. Compounds 25a-e, 25g-h, 25i-m and 25o-q were obtained by reaction of intermediates 23 and 24 with various commercially available substituted benzenesulfonamides following a similar prepare procedure to that of compound 12e. Hydrogenation of compounds 25e and 25m produced compounds 25f and 25n, respectively. Compounds 25r-v were prepared from compound 25j via the C-N coupling reaction under the palladium catalyzed conditions.

Scheme 4. Synthesis of compound 25a-v with modification on the P3 moiety^{a}

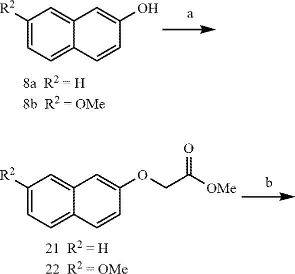

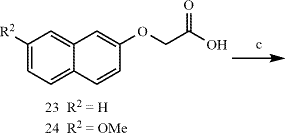

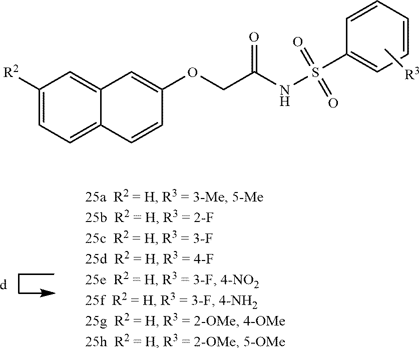

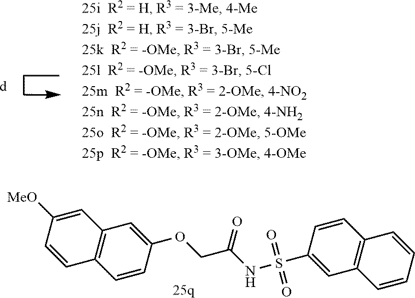

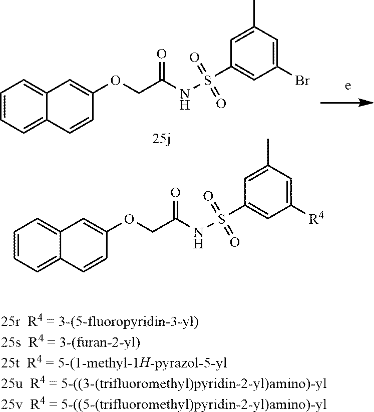

^{a}Reagents and conditions: (a) methyl 2-bromoacetate, K₂CO₃, dry DMF, rt, overnight, 84-87%; (b) i) LiOH, THF, H₂O, rt, overnight; ii) 4N HCl, 86-88%; (c) substituted benzenesulfonamide, EDCI, DAMP, DMF, rt, overnight, 39-87%; (d) Pd/C, H₂, MeOH, 50° C., 3 h, 92-94%; (e) for 25a-c, R⁴ B(OH)₂, Pd(dppf)Cl₂, K₂CO₃, 1,4-dioxane, H₂O, 110° C., overnight, 63-79%; for 25u and 25v, R⁴H, Pd(OAc)₂, XantPhos, K₂CO₃, 1,4-dioxane, 100° C., overnight, 50-72%.

Additional analogues were prepared via a different synthetic route (Scheme 5); sulfonamides 26-27 were prepared from the corresponding sulfonyl chlorides by treatment with aqueous ammonia. Biarylsulfonamides 30 and 31 were prepared from a precursor bromosulfonamide via Suzuki-Miyaura coupling. Compounds 25w-ad were then synthesized from naphthoxy acid 23 and the corresponding sulfonamides using a carbodiimide coupling.

Scheme 5. Synthesis of compound 25w-ad with modification on the P3 moiety^{a}

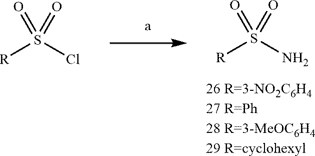

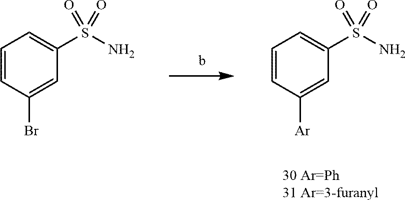

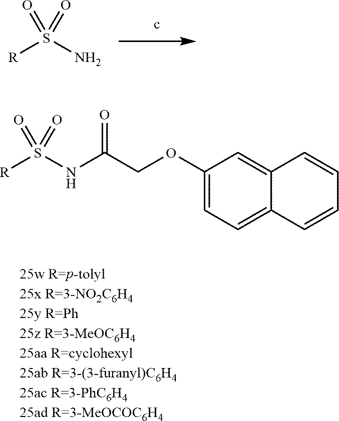

^{a}Reagents and conditions: (a) NH₄OH(_{aq}), THF, rt, 16 h, 51-96%; (b) PhB(OH)₂ or 3-furanylB(OH)₂, Pd(PPh₃)₂Cl₂, K₂CO₃, 1,4-dioxane, reflux, 16 h, 81-93%; (c) 23, EDCI, DMAP, CH₂Cl₂, rt, 48 h, 8-77%.

One aspect of the invention pertains to generally to use of compounds of the invention to selectively activate EPAC1 in cells.

### EXAMPLES

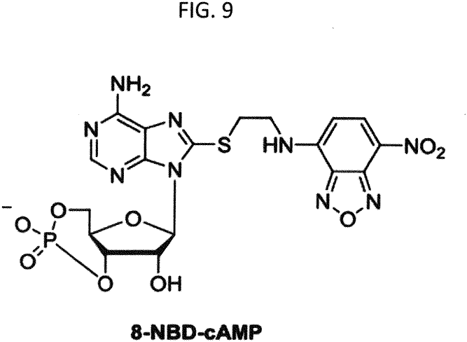

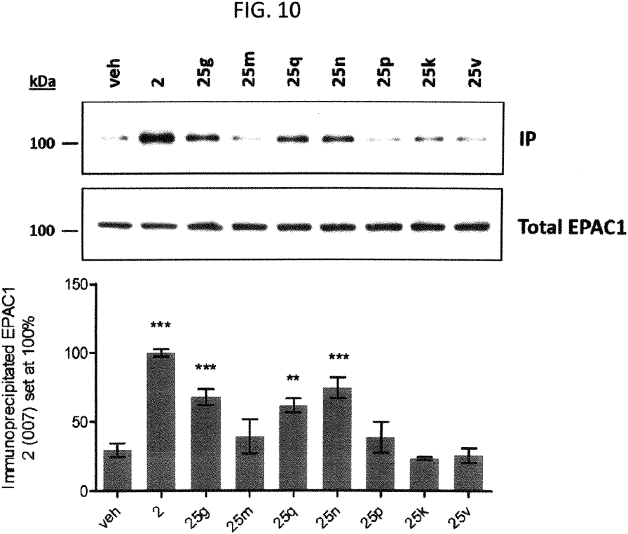

Biochemical Evaluation of EPAC1 Binding and SAR Studies. All the final target compounds have been evaluated for their binding to recombinant forms of either the isolated EPAC1 CNBD (EPAC1-CNBD) or a truncated version of the full-length protein that contains the CNBD, but lacks the N-terminal DEP domain (EPAC1-ΔDEP) using a fluorescence-based competition assay,^{49,}⁵⁰ and screening hit 3 was used as the reference compound.⁵¹ EPAC binders compete with the fluorescent ligand 8-NBD-cAMP (FIG. 9),⁵⁰ and by displacing it from the protein binding pocket, they reduce its fluorescence. Therefore, relative fluorescence intensity (RFI, described in Experimental section) is used to indicate the affinity of the final target compounds binding with EPAC1 and the results are shown in Tables 1-4. Hit compounds were subsequently tested for EPAC1 binding in a cell-based model, using EPAC1 immunoprecipitation with an activation-selective antibody (as described in Experimental section). Compounds which interacted with EPAC1 in cells were chosen for further studies (FIG. 10). We previously reported a series of 2,4,6-trimethylbenzenesulfonamide derivatives as potent and selective EPAC2 antagonists.²² Therefore, compound 9a initially designed by replacing the m-xylyl group of compound 3 with 2,4,6-trimethylbenzene group, assuming that it might enhance EPAC1 binding affinity. As shown in Table 1, the results indicate that compound 9a has a similar affinity for EPAC 1 as compound 3. Further modification of compound 9a by changing the substituted position (9b), adding a nitrogen atom (9c) or appending an acetyl group (9d) onto the naphthalene ring showed no significant improvement on the binding potency compared to compound 3. However, compound 9e, with a methoxy substitution at the 7-position of the naphthalene ring of 9a, exhibited about 1.7-fold improvement in binding potency, when compared to 9a. Reduction of the naphthalene ring of 9a into tetrahydronaphthane ring (9f) or dihydroquinolin-2(1H)-one ring (9g) showed slightly decreased binding potency. However, replacement of the P1 moiety with various bicyclic or heterocyclic rings (9h-m) resulted in a loss of the EPAC1 binding potency. These results suggest that the substitution position on the naphthalene ring of 9a is very important for EPAC1 binding affinity. In addition, adding an electron-donating group to the 7-position on the naphthalene ring of 9a benefits the EPAC1 affinity. However, replacing the naphthalene ring of 9a with other bicyclic ring and heterocyclic ring was found not favorable.

TABLE 1

EPAC binding affinities of compounds 9a-m with modifications on the P1 moiety

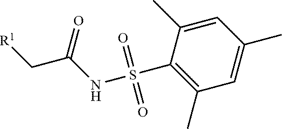

| Compd | R¹ | RFI (%)^{a} | Compd | R¹ | RFI (%)^{a} |
|---|---|---|---|---|---|
| 9a | 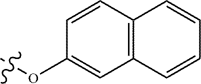 | 81 ± 1 | 9h | 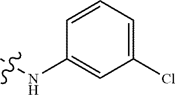 | 88 ± 9 |
| 9b | 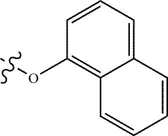 | 94 ± 5 | 9i | 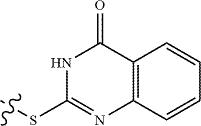 | 95 ± 4 |
| 9c | 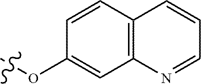 | 92 ± 2 | 9j | 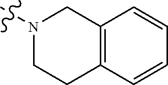 | 102 ± 1 |
| 9d | 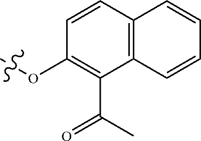 | 104 ± 7 | 9k | 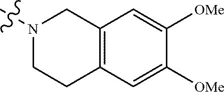 | 102 ± 5 |
| 9e | 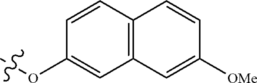 | 49 ± 3 | 9l | 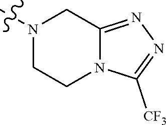 | 104 ± 2 |
| 9f | 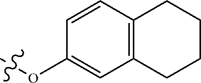 | 77 ± 2 | 9m | 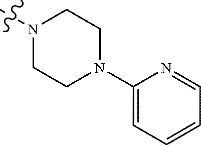 | 119 ± 8 |
| 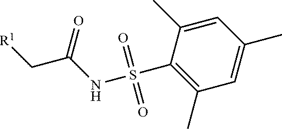 | | | | | |
| 9g | 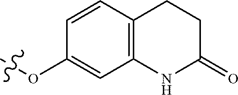 | 77 ± 4 | 3 | | 61 ± 3 |

^{a}The relative fluorescence intensity (RFI) values are the mean ± SEM of at least three independent experiments.

A further short series of compounds exploring the P1 moiety was prepared and tested for their interaction with the EPAC1 CNBD while maintaining the m-xylyl ring of 3 (Table 2). Thus, chloro- and fluoro-naphthyl analogues 9n-p, 9q and 3 were found to display similar affinity. [00183] Table 2. EPAC1 binding activities of m-xylyl compounds 9n-s with modifications on the P1 moiety

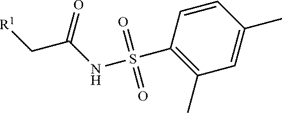

| Compd | R¹ | RFI (%)^{a} | Compd | R¹ | RFI (%)^{a} |
|---|---|---|---|---|---|
| 9n | 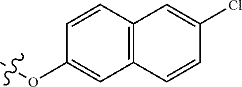 | 50 ± 2 | 9q | 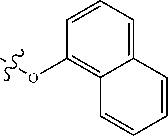 | 71 ± 1 |
| 9o | 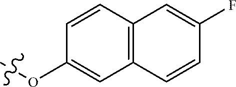 | 55 ± 1 | 9r | 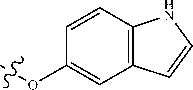 | 91 ± 2 |
| 9p | 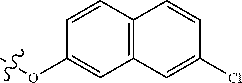 | 66 ± 2 | 9s | 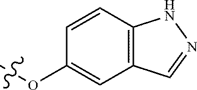 | 94 ± 4 |

^{a}The relative fluorescence intensity (RFI) values are the mean ± SEM of at least three independent experiments.

To further explore the SAR of the P2 moiety of 9a, compounds 12a-g were synthesized to probe the impact of linker on EPAC binding (Table 3). Interestingly, all these interventions (with the exception of 12e) were found to completely inhibit EPAC binding potency, even in the case of replacing the oxygen atom with its bioisostere sulfur atom (12a). Increased P2 steric bulk (12d, 12f) as well as a significant reduction in the pKa of the 3 N-acylsulfonamide proton (12c, 12d, 12g) resulted in a partial or complete loss of binding activity, in line with our previously postulated binding modes, ⁴⁶ which suggest that the acidic N-acylsulfonamide motif of 3 occupies a similar volume to the cAMP phosphate, and that the oxymethylene unit threads a narrow solvent channel.⁴⁶ These findings suggest that significant modifications on the P2 moiety of 9a are not amenable for EPAC binding enhancement.

TABLE 3

EPAC1 binding activities of compounds 12a-g with modification on the P2 moiety

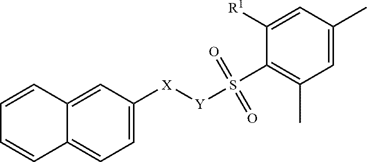

| Compd | R¹ | X | Y | RFI (%)^{a} |
|---|---|---|---|---|
| 12a | Me | S | 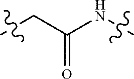 | 92 ± 2 |
| 12b | Me | NH | 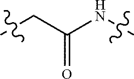 | 97 ± 5 |
| 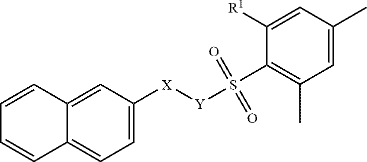 | | | | |
| 12c | Me | O | 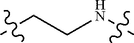 | 105 ± 4 |
| 12d | Me | O | 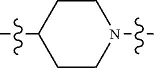 | 105 ± 3 |
| 12e | Me | O | 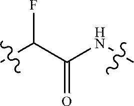 | 82 ± 4 |
| 12f | H | O | 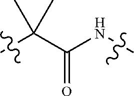 | 76 ± 3 |
| 12g | H | O | 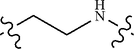 | 81 ± 3 |
| 3 | H | O | 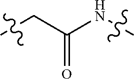 | 61 ± 3 |

^{a}The values are the mean ± SEM of at least three independent experiments.

TABLE 4

EPAC1 binding activities of compounds 25a-25ad with modifications on the P3 moiety

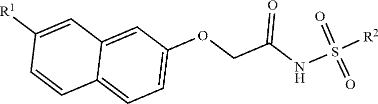

| Compd | R¹ | R² | RFI (%)^{a} |
|---|---|---|---|
| 25a | H | 3,5-dimethylphenyl | 38 ± 2 |
| 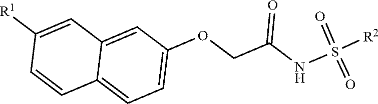 | | | |
| 25b | H | 2-fluorophenyl | 69 ± 2 |
| 25c | H | 3-fluorophenyl | 82 ± 4 |
| 25d | H | 4-fluorophenyl | 81 ± 4 |
| 25e | H | 3-fluoro-4-nitrophenyl | - |
| 25f | H | 3-fluoro-4-aminophenyl | 81 ± 8 |
| 25g | H | 2,4-dimethoxyphenyl | 23 ± 4 |
| 25h | H | 2,5-dimethoxyphenyl | 72 ± 4 |
| 25i | H | 3,4-dimethoxyphenyl | 50 ± 3 |
| 25j | H | 3-bromo-5-methylphenyl | 58 ± 2 |
| 25k | OMe | 3-bromo-5-methylphenyl | 29 ± 8 |
| 25l | OMe | 3,5-dichlorophenyl | 54 ± 6 |
| 25m | OMe | 2-methoxy-4-nitrophenyl | 24 ± 2 |
| 25n | OMe | 2-methoxy-4-aminophenyl | 7 ± 1 |
| 25o | OMe | 2,5-dimethxoylphenyl | 49 ± 4 |
| 25p | OMe | 3,4-dimethoxyphenyl | 23 ± 4 |
| 25q | OMe | 2-naphthyl | 14 ± 3 |
| 25r | H | 3-(5-fluoropyridin-3-yl)-5-methylphenyl | 36 ± 3 |
| 25s | H | 3 -(furan-2 -yl)-5-methylphenyl | 40 ± 1 |
| 25t | H | 3-methyl-5-(1-methyl-1H-pyrazol-5-ylphenyl | 34 ± 5 |
| 25u | H | 3-methyl5-(3-(trifluoromethyl)pyridin-2-yl)aminophenyl | 56 ± 6 |
| 25v | H | 3-methyl5-(5-(trifluoromethyl)pyridin-2-yl)aminophenyl | 20 ± 3 |
| 25w | H | 4-methylphenyl | 44 ± 2 |
| 25x | H | 3-nitrophenyl | 67 ± 3 |
| 25y | H | Phenyl | 49 ± 2 |
| 25z | H | 3-methoxyphenyl | 33 ± 3 |
| 25aa | H | Cyclohexyl | 81±3 |
| 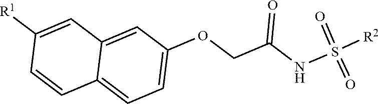 | | | |
| 25ab | H | 3-(3-furanyl)phenyl | 53 ± 2 |
| 25ac | H | 3-biphenyl | 35± 1 |
| 25ad | H | Methyl 3-benzoyl | 71 ± 1 |
| 3 | H | 2,4-dimethylphenyl | 61 ± 3 |

^{a}The values are the mean ± SEM of at least three independent experiments. “-” means not detected.

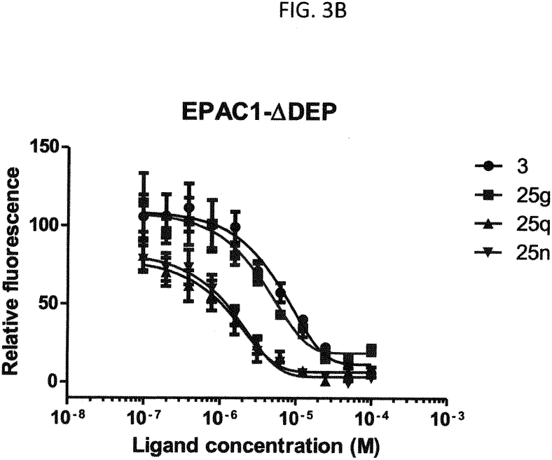

As listed in Table 4, a series of benzenesulfonamide derivatives with different substitution patterns and electronic properties were synthesized to explore the importance of the P3 moiety for EPAC1 binding affinity. Moving the dimethyl group from 2,4-position to 3,5-position, about 1.6-fold binding potency increase was observed (25a vs 3). Adding electron withdrawing groups was not tolerated and decreased the binding potency (3 vs 25b, 25c and 25d), while sterically similar benzenesulfonamide derivatives with electron donating substitutes were also investigated (3 vs 25g, 25h and 25i). It was found that compound 25 g was much more potent than the screening hit (3) with an IC₅₀ of 4.8 µM for the EPAC1-CNBD and an IC₅₀ of 4.9 µM for EPAC1-ΔDEP (aa. 149-881; FIG. 3 and Table 5). As shown in Table 1, adding an electron donating substitute at 7-position on the naphthalene ring could improve the binding potency and this conclusion was further validated by comparing 25k with 25j. Having identified that the electron donating substitutes at 7-position on the naphthalene ring and benzenesulfonamide maintain good binding potency, we then designed a series of compounds with electron donating substitutes on both sides (25k-p) were prepared, all of which displayed increased binding potency. Compound 25n was shown to be the best compound among this series with IC₅₀ values reaching sub-micromolar binding potency for both the EPAC1-CNBD and EPAC1-ΔDEP (IC₅₀ = 0.9 µM and IC₅₀ = 0.6 µM, respectively, FIG. 3 and Table 5). Interestingly, compound 25q with naphthalene rings on both sides also displays significantly enhanced binding potency, with EPAC1-CNBD binding potency (IC₅₀ = 2.2 µM, FIG. 3 and Table 5) at low micromolar level and EPAC1-full activity achieved sub-micromolar binding potency (IC₅₀ = 0.5 µM, FIG. 3 and Table 5). This result suggests that substituents with larger size (e.g. pyridine ring) on the benzenesulfonamide may be tolerated. For additional SAR studies, a heterocyclic or phenyl ring was added onto the benzene ring of 3 with or without substitutes leading to compounds 25r-v, 25ab and 25ac (Table 4). The binding potency of these compounds increased by adding the heterocyclic ring on the benzene ring of 3. Especially, compound 25v exhibited about 3-fold increased binding potency higher than the lead compound 3. Altogether, compounds with electron donating substituents at the benzene ring of 3 exhibited more favorable binding properties than compounds with electron withdrawing groups. Non-aromatic analogues of the 3 m-xylyl ring (25aa) were also investigated; replacement with a cyclohexyl ring resulted in a complete loss of affinity. Meanwhile, an additional electron donating substituent at 7-position on the naphthalene ring of 3 further improved the binding potency. Moreover, compounds which have naphthalene rings on both sides or a heterocyclic ring substituent on the benzene ring, showed positive results for the binding potency improvement.

TABLE 5

IC₅₀ values of selected EPAC1 activators for EPAC1-CNBD and EPAC1-ΔDEP

| Compound | EPAC1-CNBD | | EPAC1-ΔDEP | |
|---|---|---|---|---|
| | IC₅₀ µm^{a} | Significance in comparison to 3 | IC₅₀ µM)^{a} | Significance in comparison to 3 |
| 3 | 12.0 ± 1.1 | – | 6.7 ± 1.5 | – |
| 25g | 4.8 ± 0.4 | ∗∗∗ | 4.9 ± 1.1 | ns |
| 25q | 2.2 ± 0.2 | ∗∗∗ | 0.5 ± 0.1 | ∗∗ |
| 25n | 0.9 ± 0.1 | ∗∗∗ | 0.6 ± 0.1 | ∗∗ |

^{a}The values are the mean ± SEM of at least three independent experiments. Significance in comparison to compound 3 was determined by one-way ANOVA with Tukey post-hoc test; ^{∗∗} p < 0.01, ^{∗∗∗} p < 0.001, “ns” means not significant.

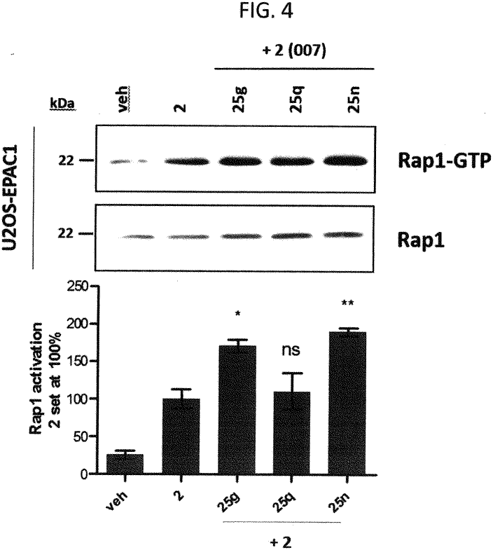

Potential of Newly Discovered EPAC1 Binders to Activate EPAC1 Given the improved affinity of 25g, 25q, and 25n for EPAC1 observed in the 8-NBD-cAMP competition assay, their ability to activate EPAC1 cells expressing EPAC1 to determine if compounds 25g, 25n and 25q can activate cellular EPAC activity was next investigated, by measuring the nucleotide loading state of Rap 1 (FIG. 4). In these assays, a selective binding protein was used to isolate active, GTP-bound Rap1 from cell extracts, which was demonstrated by western blotting as described in the Experimental Procedures section. Experiments were carried out in the presence of the EPAC1-selective cAMP analogue 2 (007), to determine whether or not 25g, 25n and 25q acted as agonists or partial agonists in cells Experiments revealed that compounds 25g and 25n enhanced, as opposed to inhibited, activation of EPAC1 by compound 2, indicating that they are acting as agonists, rather than partial agonists in cells (FIG. 4). Together these results indicate that 25g and 25n and 25q bind strongly to EPAC1 in binding assays and activate EPAC1 in cells.

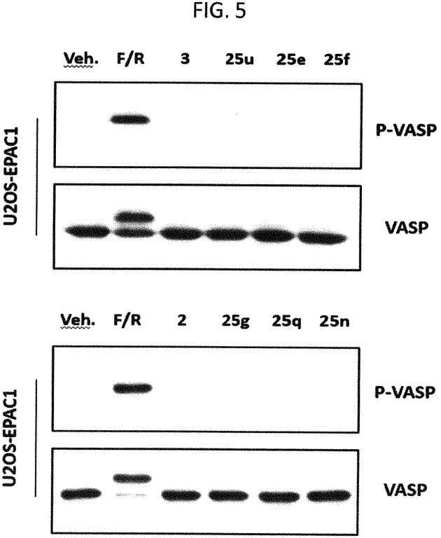

PKA and GPCR Selectivity. To investigate the EPAC selectivity of these EPAC agonists, compounds 25g, 25n and 25q were selected for further PKA activation studies, and the results are shown in FIG. 5. PKA activation was assessed by monitoring phosphorylation state of a downstream PKA effector, vasodilator-stimulated phosphoprotein (VASP). In western blot studies, these three compounds did not induce any PKA activation. This result suggests our newly designed EPAC agonists have excellent EPAC/PKA selectivity, without potential PKA activation side effects. Additionally, compounds 25n and 25q were further chosen for the counter screening study of over 40 GPCR targets.⁵² In vitro functional selectivity profiles of these three compounds were investigated for their affinity across a broad panel of over 40 GPCR proteins (Supporting Information, Table S1), indicating that these EPAC agonists are highly specific and none of them displays the potential off-target effects towards these tested GPCR proteins.

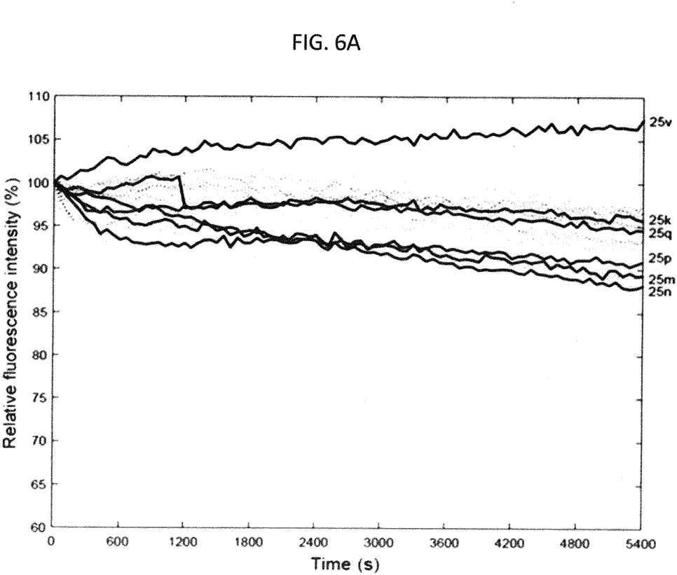

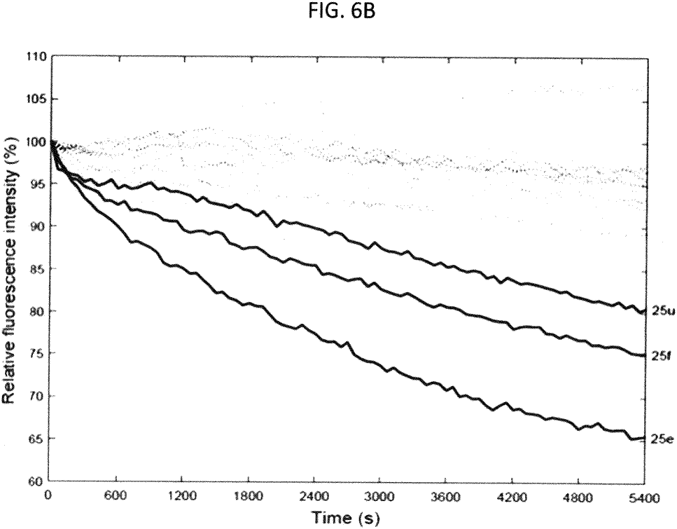

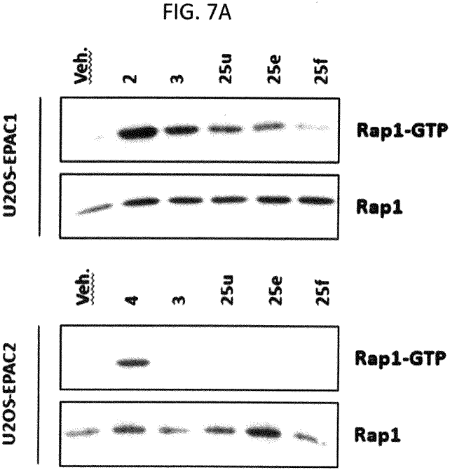

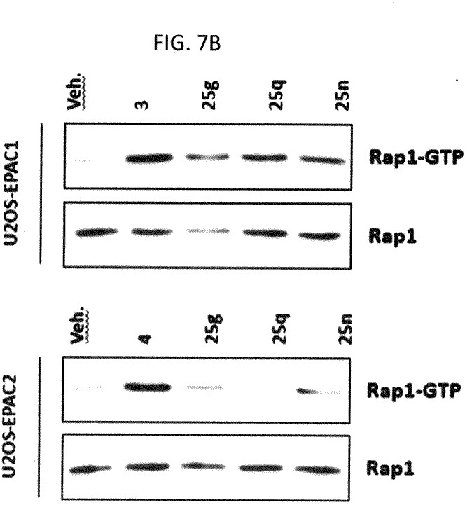

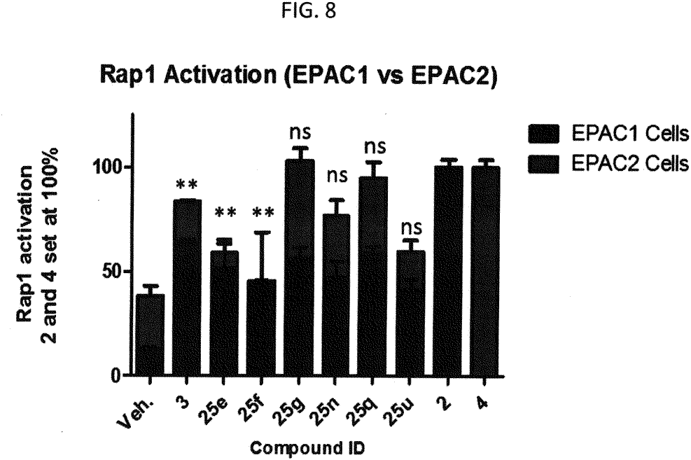

Series Expansion and EPAC1 vs EPAC2 Selectivity. GEF assay was used to further screen all newly synthesis analogues at 10 µM, to identify additional activating compounds to expand the series of EPAC1 agonists (FIG. 6). Using this approach three more compounds, 25e, 25f and 25u were discovered that appeared to promote EPAC1 activity more effectively than 25g, 25n and 25q (FIG. 6) and were unable to activate PKA (FIG. 6). Having now identified an expanded exemplary series of compounds as potential EPAC1 activators the ability of 25e, 25f, 25g, 25n and 25u to activate EPAC1 and EPAC2 in cellular Rap1 activation assays (FIG. 7) were compared. For these U2OS cells transfected with either EPAC1 or EPAC2 (FIG. 7) was used. From these assays it was found that all compounds in the series induced Rap1 activation in EPAC1 cells, with compounds 25e and 25u promoting levels of activation roughly equivalent to the positive control, compound 3 (FIG. 7). Densitometry was carried out on all immunoblots and the ratio of EPAC1 to EPAC2 activation was calculated and presented in a bar graph in FIG. 8. Results demonstrated that compounds 25e and 25f demonstrated the best selectivity, in terms of their ability to activate EPAC1 over EPAC2.

### Experimental Section

General. All commercially available starting materials and solvents were reagent grade and used without further purification. Reactions were performed under a nitrogen atmosphere in dry glassware with magnetic stirring. Preparative column chromatography was performed using silica gel 60, particle size 0.063-0.200 mm (70-230 mesh, flash). Analytical TLC was carried out employing silica gel 60 F254 plates (Merck, Darmstadt). Visualization of the developed chromatograms was performed with detection by UV (254 nm). NMR spectra were recorded on a Bruker-600 or AV300 (¹H, 300 MHz; ¹³C, 75.5 MHz) or Bruker AV400 (¹H, 400 MHz, ¹³C, 101 MHz) spectrometer. ¹H and ¹³C NMR spectra were recorded with TMS as an internal reference or referenced to solvent. Chemical shifts downfield from TMS were expressed in ppm, and J values were given in Hz. High-resolution mass spectra (HRMS) were obtained from Thermo Fisher LTQ Orbitrap Elite mass spectrometer or form the EPSRC UK National Mass Spectrometry Facility at Swansea University. Parameters include the following: nano ESI spray voltage was 1.8 kV, capillary temperature was 275° C., and the resolution was 60000; ionization was achieved by positive mode. Purity of final compounds was determined by analytical HPLC, which was carried out on a Shimadzu HPLC system (model: CBM-20A LC-20AD SPD-20A UV/vis). HPLC analysis conditions: Waters µBondapak C18 (300 mm × 3.9 mm), flow rate 0.5 mL/min, UV detection at 270 and 254 nm, linear gradient from 10% acetonitrile in water (0.1% TFA) to 100% acetonitrile (0.1% TFA) in 20 min, followed by 30 min of the last-named solvent. All biologically evaluated compounds are >95% pure.

2,4,6-Trimethylbenzenesulfonamide (6). To a solution of 2,4,6-trimethylbenzenesulfonyl chloride (5) (1.1 g, 5 mmol) in THF (10 mL) was added 35% NH₄OH(_{aq}) (3.5 mL). The mixture was stirred at room temperature overnight, and then added with 20 mL water and then extracted with EtOAc (15 mL × 2). The combined EtOAc extracts were successively washed with brine, then dried with Na₂SO₄, filtered, and concentrated to give the desired compound 5 as a white solid (0.94 g, 94%). ¹H NMR (300 MHz, Chloroform-d) δ 6.99 (s, 2H), 4.81 (s, 2H), 2.68 (s, 6H), 2.33 (s, 3H).

2-Bromo-N-(mesitylsulfonyl)acetamide (7). Compound 6 (1.0 g, 5 mmol) was dissolved in 25 mL dry toluene and stirred at room temperature. Bromoacetyl bromide (1.7 mL, 20 mmol) was added dropwise to the reaction mixture and it was stirred at reflux for 5 hours. After 5 hours the reaction mixture was cooled to room temperature and then placed on an ice. The product crystalized out of the toluene and was collected by vacuum filtration and rinsed with cold toluene. Compound 7 was obtained as a gray solid (1.1 g, 67%). ¹H NMR (300 MHz, Methanol-d₄) δ 7.05 (d, J= 0.6 Hz, 2H), 3.81 (s, 2H), 2.69 (s, 6H), 2.33 (s, 3H).

N-(Mesitylsulfonyl)-2-(naphthalen-2-yloxy)acetamide (9a). To a solution of 7 (64 mg, 0.2 mmol) in dry DMF (1 mL) was added K₂CO₃ (55 mg, 0.4 mmol) and naphthalen-2-ol (29 mg, 0.2 mmol). The mixture was stirred at room temperature overnight, added with 5 mL water and then extracted with EtOAc (10 mL × 3). The combined EtOAc extracts were successively washed with brine, then dried with Na₂SO₄, filtered, and concentrated to the residue. This residue was further purified by preparative TLC plates (CH₂Cl₂/MeOH = 50:1) to produce compound 9a as a white solid (49 mg, 68%). ¹H NMR (300 MHz, Chloroform-d) δ 9.14 (s, 1H), 7.82 (d, J= 8.5 Hz, 2H), 7.65 (d, J= 8.0 Hz, 1H), 7.54 - 7.39 (m, 2H), 7.20 (dd, J = 9.0, 2.6 Hz, 1H), 7.00 (d, J = 2.6 Hz, 1H), 6.91 (s, 2H), 4.59 (s, 2H), 2.61 (s, 6H), 2.32 (s, 3H). ¹³C NMR (75 MHz, Chloroform-d) δ 166.8, 154.4, 144.0, 140.7, 134.1, 132.1, 132.0, 130.3, 129.7, 127.7, 127.0, 126.8, 124.7, 117.9, 107.6, 67.3, 23.0, 21.1. HRMS (ESI) calcd for C₂₁H₂₁NO₄SNa 406.1089 (M + Na)⁺, found 406.1068.

N-(Mesitylsulfonyl)-2-(naphthalen-1-yloxy)acetamide (9b). Following the synthetic procedure of compound 9a, compound 9b was obtained as a white solid (48 mg, 67%). ¹H NMR (300 MHz, Chloroform-d) δ 9.07 (s, 1H), 8.27 - 8.14 (m, 1H), 7.93 - 7.82 (m, 1H), 7.67 - 7.52 (m, 3H), 7.34 (t, J= 8.0 Hz, 1H), 6.99 (s, 2H), 6.69 (d, J = 7.7 Hz, 1H), 4.68 (s, 2H), 2.64 (s, 6H), 2.34 (s, 3H). ¹³C NMR (75 MHz, Chloroform-d) δ 166.8, 152.3, 144.0, 140.7, 134.7, 132.1, 127.9, 127.0, 126.2, 125.4, 125.0, 122.7, 121.0, 105.9, 67.8, 22.67, 21.1. HRMS (ESI) calcd for C₂₁H₂₁NO₄SNa 406.1089 (M + Na)⁺, found 406.1068.

N-(Mesitylsulfonyl)-2-(quinolin-7-yloxy)acetamide (9c). Following the synthetic procedure of compound 9a, compound 9c as a white solid (38 mg, 48%). ¹H NMR (300 MHz, Chloroform-d) δ 8.87 (dd, J = 4.5, 1.7 Hz, 1H), 8.12 (dd, J = 8.2, 1.7 Hz, 1H), 7.76 (d, J= 9.0 Hz, 1H), 7.40 - 7.31 (m, 2H), 7.26 (dd, J = 9.0, 2.5 Hz, 1H), 6.95 (s, 2H), 6.35 (s, 1H), 4.64 (s, 2H), 2.68 (s, 6H), 2.31 (s, 3H). ¹³C NMR (75 MHz, Chloroform-d) δ 166.3, 157.4, 150.8, 149.1, 144.0, 140.6, 136.0, 132.1, 129.6, 124.4, 119.9, 119.0, 108.9, 67.1, 22.7, 21.1. HRMS (ESI) calcd for C₂₀H₂₀N₂O₄SNa 407.1041 (M + Na)⁺, found 407.1025.

2-((1-Acetylnaphthalen-2-yl)oxy)-N-(mesitylsulfonyl)acetamide (9d). Following the synthetic procedure of compound 9a, compound 9d as a white solid (52 mg, 61%). ¹H NMR (300 MHz, Chloroform-d) δ 10.33 (s, 1H), 7.96 -7.85 (m, 2H), 7.73 (dd, J = 8.4, 1.1 Hz, 1H), 7.60 (ddd, J = 8.4, 6.8, 1.4 Hz, 1H), 7.50 (ddd, J = 8.1, 6.8, 1.3 Hz, 1H), 7.15 (d, J = 9.1 Hz, 1H), 6.92 (s, 2H), 4.69 (s, 2H), 2.77 (s, 3H), 2.61 (s, 6H), 2.29 (s, 3H). ¹³C NMR (75 MHz, Chloroform-d) δ 205.5, 150.4, 143.6, 140.7, 132.4, 131.9, 130.5, 130.0, 129.7, 128.6, 128.3, 126.5, 125.4, 123.7, 113.8, 68.7, 32.9, 22.5, 21.0. HRMS (ESI) calcd for C₂₃H₂₃NO₅SNa 448.1195 (M + Na)⁺, found 448.1180.

N-(Mesitylsulfonyl)-2-((7-methoxynaphthalen-2-yl)oxy)acetamide (9e). Following the synthetic procedure of compound 9a, compound 9e as a white solid (46 mg, 56%). ¹H NMR (300 MHz, Chloroform-d) δ 9.06 (s, 1H), 7.72 (t, J = 8.6 Hz, 2H), 7.09 (dd, J = 8.9, 2.5 Hz, 1H), 7.04 (dd, J = 8.9, 2.6 Hz, 1H), 6.96 (dd, J = 9.4, 2.6 Hz, 2H), 6.92 (s, 2H), 4.58 (s, 2H), 3.92 (s, 3H), 2.61 (s, 6H), 2.31 (s, 3H). ¹³C NMR (75 MHz, Chloroform-d) δ 166.8, 158.6, 155.0, 143.9, 140.7, 135.6, 132.0, 130.0, 129.2, 125.1, 117.3, 115.1, 107.0, 105.5, 67.3, 55.3, 22.7, 21.0. HRMS (ESI) calcd for C₂₂H₂₃NO₅SNa 436.1195 (M + Na)⁺, found 436.1175.

N-(Mesitylsulfonyl)-2-((5,6,7,8-tetrahydronaphthalen-2-yl)oxy)acetamide (9f). Following the synthetic procedure of compound 9a, compound 9f as a white solid (42 mg, 54%). ¹H NMR (300 MHz, Chloroform-d) δ 9.01 (s, 1H), 7.05 - 6.96 (m, 3H), 6.65 (dd, J = 8.4, 2.8 Hz, 1H), 6.56 (d, J = 2.7 Hz, 1H), 4.43 (s, 2H), 2.72 (d, J = 5.4 Hz, 4H), 2.66 (s, 6H), 2.33 (s, 3H), 1.87 -1.74 (m, 4H), 1.58 (s, 4H). ¹³C NMR (75 MHz, Chloroform-d) δ 167.0, 154.3, 143.9, 140.7, 138.9, 132.0, 131.7, 130.4, 114.5, 112.4, 67.4, 29.6, 28.6, 23.2, 23.0, 22.7, 21.1. HRMS (ESI) calcd for C₂₁H₂₅NO₄SNa 410.1402 (M + Na)⁺, found 410.1386.

N-(Mesitylsulfonyl)-2-((2-oxo-1,2,3,4-tetrahydroquinolin-6-yl)oxy)acetamide (9 g). Following the synthetic procedure of compound 9a, compound 9 g as a white solid (38 mg, 48%). ¹H NMR (300 MHz, DMSO-d₆) δ 12.41 (s, 1H), 9.90 (s, 1H), 7.05 (s, 2H), 6.71 (d, J = 8.3 Hz, 1H), 6.59 (d, J = 7.8 Hz, 2H), 4.56 (s, 2H), 2.80 - 2.73 (m, 2H), 2.59 (s, 6H), 2.38 (dd, J = 8.5, 6.4 Hz, 2H), 2.27 (s, 3H). ¹³C NMR (75 MHz, DMSO-d₆) δ 170.1, 168.2, 153.1, 143.4, 140.1, 133.5, 132.9, 132.0, 125.2, 116.1, 114.4, 113.5, 66.8, 30.7, 25.5, 22.5, 20.9. HRMS (ESI) calcd for C₂₀H₂₂N₂O₅SNa 425.1147 (M + Na)⁺, found 425.1134.

2-((3-Chlorophenyl)amino)-N-(mesitylsulfonyl)acetamide (9h). Following the synthetic procedure of compound 9a, compound 9h as a white solid (41 mg, 56%). ¹H NMR (300 MHz, Chloroform-d) δ 9.24 (s, 1H), 7.11 (t, J = 7.9 Hz, 1H), 6.99 (s, 2H), 6.88 - 6.81 (m, 1H), 6.47 - 6.38 (m, 2H), 4.43 (s, 1H), 3.79 (d, J = 3.3 Hz, 2H), 2.57 (s, 6H), 2.34 (s, 3H). ¹³C NMR (75 MHz, Chloroform-d) δ 169.2, 147.2, 144.0, 140.6, 135.5, 132.1, 131.9, 130.6, 119.9, 113.0, 111.7, 48.7, 22.6, 21.1. HRMS (ESI) calcd for C₁₇H₁₉ClN₂O₃SNa 389.0703 (M + Na)⁺, found 389.0688.

N-(Mesitylsulfonyl)-2-((4-oxo-3,4-dihydroquinazolin-2-yl)thio)acetamide (9i). Following the synthetic procedure of compound 9a, compound 9i as a white solid (33 mg, 79%). ¹H NMR (300 MHz, Methanol-d₄) δ 8.10 (dd, J= 8.2, 1.6 Hz, 1H), 7.74 (t, J = 7.6 Hz, 1H), 7.43 (td, J= 6.6, 6.1, 2.9 Hz, 2H), 6.90 (s, 2H), 3.99 (s, 2H), 2.65 (s, 6H), 2.20 (s, 3H). ¹³C NMR (75 MHz, Methanol-d₄) δ 167.6, 166.6, 148.3, 143.2, 140.1, 134.4, 132.9, 131.4, 125.8, 119.6, 47.3, 47.0, 46.7, 34.0, 21.5, 19.6. HRMS (ESI) calcd for C₁₉H₁₉N₃O₄S₂Na 440.0715 (M + Na)⁺, found 440.0698.

2-(3,4-Dihydroisoquinolin-2(1H)-yl)-N-(mesitylsulfonyl)acetamide (9j). Following the synthetic procedure of compound 9a, compound 9j as a white solid (35 mg, 82%). ¹H NMR (300 MHz, Chloroform-d) δ 7.23 - 7.16 (m, 4H), 7.02 (d, J = 8.1 Hz, 3H), 3.73 (s, 2H), 3.20 (s, 2H), 3.00 (t, J = 6.0 Hz, 2H), 2.86 (t, J = 5.9 Hz, 2H), 2.70 (s, 6H), 2.34 (s, 3H). ¹³C NMR (75 MHz, Chloroform-d) δ 169.0, 143.6, 140.5, 133.0, 132.0, 128.8, 126.8, 126.4, 126.1, 61.3, 56.0, 51.5, 28.8, 22.8, 21.1. HRMS (ESI) calcd for C₂₀H₂₅N₂O₃S 373.1586 (M + H)⁺, found 373.1574.

2-(6,7-Dimethoxy-3,4-dihydroisoquinolin-2(1H)-yl)-N-(mesitylsulfonyl)acetamide (9k). Following the synthetic procedure of compound 9a, compound 9k as a white solid (38 mg, 81%). ¹H NMR (300 MHz, Chloroform-d) δ 6.99 (s, 2H), 6.64 (s, 1H), 6.50 (s, 1H), 3.89 (s, 3H), 3.87 (s, 3H), 3.67 (s, 2H), 3.18 (s, 2H), 2.87 (dd, J = 10.8, 4.6 Hz, 4H), 2.70 (s, 6H), 2.33 (s, 3H). ¹³C NMR (75 MHz, Chloroform-d) δ 169.1, 148.1, 147.6, 143.6, 140.4, 132.6, 132.0, 125.0, 124.8, 111.5, 109.3, 61.1, 56.0, 56.0, 55.6, 51.5, 28.2, 22.8, 21.1. HRMS (ESI) calcd for C₂₂H₂₉N₂O₅S 433.1797 (M + H)⁺, found 433.1782.

N-(Mesitylsulfonyl)-2-(3-(trifluoromethyl)-5,6-dihydro-[1,2,4]triazolo[4,3-a]pyrazin-7(8H)-yl)acetamide (9l). Following the synthetic procedure of compound 9a, compound 9l as a white solid (146 mg, 70%). ¹H NMR (300 MHz, Chloroform-d) δ 9.85 (s, 1H), 7.00 (s, 2H), 4.24 (t, J = 5.6 Hz, 2H), 4.01 (s, 2H), 3.37 (s, 2H), 3.15 - 3.04 (m, 2H), 2.67 (s, 6H), 2.33 (s, 3H). ¹³ C NMR (75 MHz, Chloroform-d) δ 167.2, 150.7, 144.1, 140.5, 132.1, 131.9, 77.2, 60.2, 49.5, 49.0, 43.1, 22.8, 21.1. HRMS (ESI) calcd for C₁₇H₂₁F₃N₅O₃S 432.1317 (M + H)⁺, found 432.1304.

N-(Mesitylsulfonyl)-2-(4-(pyridin-2-yl)piperazin-1-yl)acetamide (9 m). Following the synthetic procedure of compound 9a, compound 9 m as a white solid (32 mg, 80%). ¹H NMR (300 MHz, Chloroform-d) δ 8.26 - 8.18 (m, 1H), 7.52 (ddd, J = 9.1, 7.5, 2.0 Hz, 1H), 7.00 (s, 2H), 6.68 (dd, J = 7.6, 4.9 Hz, 2H), 3.62 (t, J = 5.0 Hz, 4H), 3.07 (s, 2H), 2.72 (s, 6H), 2.65 (t, J = 5.0 Hz, 4H), 2.32 (s, 3H). ¹³C NMR (75 MHz, Chloroform-d) δ 168.6, 159.1, 148.0, 143.7, 140.4, 137.6, 132.4, 132.0, 113.9, 107.2, 61.7, 53.3, 45.2, 22.8, 21.1. HRMS (ESI) calcd for C₂₀H₂₇N₄O₃S 403.1804 (M + H)⁺, found 403.1788.

2,4-Dimethylbenzenesulfonamide (11). 18.1 M NH₄OH(_{aq}) (21.8 mL, 21.9 mmol) was added dropwise to a stirred solution of 2,4-dimethylbenzenesulfonyl chloride (3.0 g, 14.6 mmol) in THF (15 mL) at 0° C. The resulting solution was allowed to warm to rt and stirred for 16 h. Water was added, and the two layers were separated. The aqueous layer was extracted with EtOAc (30 × 3). The combined organic layers were dried (MgSO₄) and evaporated under reduced pressure to give 11 as a white solid (2.5 g, 93%); 137-139° C.; IR (solid) 3361 (N-H str), 3253 (N-H str), 1314, 1294, 1172, 1154, 1133, 823 cm⁻¹; ¹H NMR (300 MHz, CDCl₃) δ 7.88 (d, J= 8.0 Hz, 1H, Ar), 7.18 - 7.04 (m, 2H, Ar), 4.83 (br s, 2H, NH₂), 2.64 (s, 3H, CH₃), 2.37 (s, 3H, CH₃); ¹³C NMR (101 MHz, CDCl₃) δ 143.7 (C), 137.2 (C), 136.8 (C), 133.3 (CH), 128.5 (CH), 126.9 (CH), 21.4 (CH₃), 20.3 (CH₃). Spectroscopic data consistent with those reported in the literature.⁵³

6-Chloronaphthalen-2-ol (8n). 6-Bromonaphthalen-2-ol (1.0 g, 4.5 mmol) in THF (10 mL) was added dropwise to a stirred suspension of NaH (900 mg 22.5 mmol) in THF (10 mL) at rt for 30 min. MOMCl (0.9 mL, 11.3 mmol) was then added, and the solution was stirred at rt for a further 2 h. The solution was then quenched sequentially with water (10 mL) and MeOH (10 mL). Et₂O was added and the layers were separated. Aqueous layer was extracted with Et₂O (3 × 20 mL). The organic layers were combined and washed with water (20 mL), brine (20 mL), and NaHCO₃(_{aq}), dried (MgSO₄), and evaporated under reduced pressure to give the crude product. Purification by flash column chromatography on silica with petroleum ether:EtOAc (90:10) as eluent gave 2-bromo-6-(methoxymethoxy)naphthalene as a white solid (721 mg, 60%); R_{F} 0.3 (petroleum ether:EtOAc 90:10); mp 65-67° C. ; IR (solid) 2956, 2926, 2852, 2826, 1621, 1586, 1495, 1478, 1464, 1252, 1217, 1196, 1151, 1124, 1076, 1061, 880, 861 cm⁻¹ ;¹H NMR (300 MHz, CDCl₃) δ 7.93 (d, J = 2.0 Hz, 1H, Ar), 7.67 (d, J = 9.0 Hz, 1H, Ar), 7.61 (d, J= 9.0 Hz, 1H, Ar), 7.50 (dd, J= 9.0, 2.0 Hz, 1H, Ar), 7.37 (d, J= 2.5 Hz, 1H, Ar), 7.23 (dd, J= 9.0, 2.5 Hz, 1H, Ar), 5.29 (s, 2H, CH₂), 3.52 (s, 3H, CH₃); ¹³C NMR (75.5 MHz, CDCl₃) δ 155.5 (C), 133.0 (C), 130.7 (C), 129.8 (CH), 129.8 (CH), 128.8 (CH), 128.7 (CH), 120.2 (CH), 117.7 (C), 110.0 (CH), 94.6 (CH₂), 56.3 (CH₃). Spectroscopic data consistent with those reported in the literature.⁵⁴

Next, n-BuLi (1.4 mL of a 2.5 M solution in hexanes, 3.38 mmol) was added to a stirred solution of 2-bromo-6-(methoxymethoxy)naphthalene (600 mg, 2.25 mmol) in THF (10 mL) at -78° C. for 30 min, then a solution of NCS (300 mg, 2.25 mmol) in THF (10 mL) was added and the solution was allowed to warm to rt and stirred for 16 h. The solution was then quenched with water (10 mL). EtOAc (20 mL) was added and the two layers were separated, extracting the aqueous with EtOAc (3 × 20 mL). The combined organic layers were washed with water (20 mL) and brine (20 mL), dried (MgSO₄) and evaporated under reduced pressure to give the crude product. Purification by flash column chromatography on silica with petroleum ether:EtOAc (99:1) as eluent gave product 2-chloro-6-(methoxymethoxy)naphthalene as a white solid (104 mg, 21%), R_{F} 0.2 (petroleum ether:EtOAc 99:1); 55-57° C. ; IR (solid) 2957, 2903, 2829, 1616, 1589, 1500, 1479, 1464, 1269, 1253, 1218, 1124, 1153, 1074, 991, 962, 956, 908 cm⁻¹; ¹H NMR (300 MHz, CDCl₃) δ 7.79 (d, J= 1.5 Hz, 1H, Ar), 7.72 - 7.69 (m, 2H, Ar), 7.44 - 7.40 (m, 2H, Ar), 7.29 (dd, J= 9.0, 2.5 Hz, 1H, Ar), 5.33 (s, 2H, CH₂), 3.57 (s, 3H, CH₃); ¹³C NMR (75.5 MHz, CDCl₃) δ 155.3 (C), 132.8 (C), 130.1 (C), 129.7 (C), 2 × 128.6 (CH and CH), 127.3 (CH), 126.4 (CH), 120.1 (CH), 110.0 (CH), 94.6 (CH₂), 56.2 (CH₃); LRMS (TOF MS ASAP+) m/z 222 ([M+H]⁺, 10), 191 ([M - OMe]⁺, 100), 157 ([M - OMe - Cl]⁺, 30); HRMS (TOF MS ASAP+) m/z C₁₂H₁₁O₂Cl ([M+H]⁺) calcd for 222.0448, found 222.0446.

Next, a solution of 2-chloro-6-(methoxymethoxy)naphthalene 3 (100 mg, 0.47 mmol) in MeOH (5 mL) was stirred and heated at 50° C. and 6 M HCl (10 drops) was added before stirring for a further 2 h. The resulting solution was allowed to cool to rt then EtOAc (20 mL) and water (10 mL) were added and the layers were separated. The organic layer was washed with water (10 mL) and brine (10 mL), dried (MgSO₄) and evaporated under reduced pressure to give the crude product. Purification by flash column chromatography on silica eluting with petroleum ether:EtOAc (90:10) gave 6-chloronaphthalen-2-ol 8n as a tan solid (77 mg, 92%); R_{F} 0.3 (petroleum ether:EtOAc 90:10); 63-66° C. ; IR (solid) 3265 (O—H), 2962, 2425, 1627, 1591, 1575, 1559, 1505, 1466, 1442, 1429, 1386, 1348, 1267, 1240, 1201, 1159, 1148, 1127, 1075, 914, 886, 876, 861, 807 cm⁻¹; ¹H NMR (300 MHz, Acetone-d₆) δ 9.01 (br s, 1H, OH), 7.81 (d, J= 2.0 Hz, 1H, Ar), 7.75 (d, J= 9.0 Hz, 1H, Ar), 7.69 (d, J= 9.0 Hz, 1H, Ar), 7.35 (dd, J= 9.0, 2.0 Hz, 1H, Ar), 7.26 - 7.17 (m, 2H, Ar); ¹³C NMR (75.5 MHz, Acetone-d₆) δ 156.5 (C), 134.2 (C), 129.8 (C), 129.6 (CH), 128.9 (CH), 128.65 (C), 127.45 (CH), 127.1 (CH), 120.4 (CH), 109.9 (CH); Spectroscopic data consistent with those reported in the literature.⁵⁵

2-((6-Chloronaphthalen-2-yl)oxy)acetic acid (10n). A solution of 6-chloronaphthalen-2-ol 8n (75 mg, 0.42 mmol), ethyl bromoacetate (56 µL, 0.5 mmol) and K₂CO₃ (120 mg, 0.84 mmol) in acetone (5 mL) was stirred and heated at reflux for 16 h. The resulting solution was allowed to cool to rt then filtered and evaporated under reduced pressure. NaOH in MeOH was added to the residue and solution was stirred at rt for 3 h. The resulting solution was evaporated under reduced pressure and then acidified with 1 M HCl. Water and EtOAc was added and the two layers were separated. The aqueous layer was extracted with EtOAc (×3). The combined organic layers were washed with brine, dried (MgSO₄) and evaporated under reduced pressure to give 10n as an off-white solid (89 mg, 89%); 176-178° C.; IR (solid) 2911 (O—H), 2586, 1733 (C═O), 1627, 1594, 1503, 1429, 1407, 1389, 1359, 1345, 1209, 1168, 1081, 881, 822 cm⁻¹; ¹H NMR (400 MHz, DMSO-d₆) δ 7.97 (d, J= 2.0 Hz, 1H, Ar), 7.85 (d, J= 4.0 Hz, 1H, Ar), 7.83 (d, J = 4.0 Hz, 1H, Ar), 7.46 (dd, J = 9.0, 2.0 Hz, 1H, Ar), 7.33 (d, J = 2.5 Hz, 1H, Ar), 7.27 (dd, J= 9.0, 2.5 Hz, 1H, Ar), 4.80 (s, 2H, CH₂); ¹³C NMR (75.5 MHz, DMSO-d₆) δ 169.92 (C), 155.96 (C), 132.49 (C), 129.28 (C), 128.83 (CH), 128.72 (CH), 128.09 (C), 126.84 (CH), 126.13 (CH), 119.67 (CH), 107.12 (CH), 64.5 (CH₂); LRMS (ESI) m/z 235 ([M]⁺, 100), 202 ([M - OH]⁺, 10); HRMS (ESI) m/z C₁₂H₉O₃Cl ([M]⁺) calcd for 235.0167, found 235.0170.

2-((6-Chloronaphthalen-2-yl)oxy)-N-((2,4-dimethylphenyl)sulfonyl)acetamide (9n). A solution of 10n (46 mg, 0.19 mmol), sulfonamide 11 (36 mg, 0.19 mmol), EDCI (45 mg, 0.23 mmol) and DMAP (24 mg, 0.19 mmol) in CH₂Cl₂ (5 mL) was stirred at rt for 72 h. Water was added and the two layers were separated. The organic layer was washed with 1 M HCl (5 mL × 3), water, brine, dried (MgSO₄) and evaporated under reduced pressure to give the crude product. Purification by recrystallization from toluene gave product 9n as a white solid (12 mg, 16%); mp 208-210° C.; IR (solid) 3269 (N—H), 1722 (C═O), 1432, 1410, 1356, 1330, 1196, 1174, 1158, 1140, 1057, 1048, 930, 911 cm⁻¹; ¹H NMR (400 MHz, DMSO-d₆) δ 7.96 (d, J = 2.0 Hz, 1H, Ar), 7.87 - 7.78 (m, 2H, Ar), 7.71 (d, J = 9.0 Hz, 1H, Ar), 7.47 (dd, J = 9.0, 2.0 Hz, 1H, Ar), 7.25 - 7.15 (m, 3H, Ar), 7.08 (d, J = 2.5 Hz, 1H, Ar), 4.77 (s, 2H, CH₂), 2.54 (s, 3H, CH₃), 2.31 (s, 3H, CH₃); ¹³C NMR (101 MHz, DMSO-d₆) δ 166.8 (C), 155.7 (C), 143.9 (C), 136.8 (C), 134.5 (C), 132.8 (CH), 132.2 (C), 130.3 (CH), 129.3 (C), 128.7 (CH), 128.6 (CH), 128.2 (C), 126.8 (CH), 126.5 (CH), 126.1 (CH), 119.5 (CH), 107.1 (CH), 66.2 (CH₂), 20.7 (CH₃), 19.4 (CH₃); LRMS (TOF MS ASAP+) m/z 404 ([M+H]⁺, 100), 219 ([M - NHSO₂C₈H₉]⁺, 10); HRMS (TOF MS ASAP+) m/z C₂₀H₁₉NO₄SCl ([M+H]⁺) calcd for 404.0723, found 404.0722.

6-Fluoronaphthalen-2-ol (8o). n-BuLi (0.8 mL of a 2.5 M solution in hexanes, 2.0 mmol) was added to a stirred solution of 2-bromo-6-(methoxymethoxy)naphthalene (prepared during the synthesis of 8o, above) (350 mg, 1.3 mmol) in THF (5 mL) at -78° C. for 30 min, then a solution of NFSI (410 mg, 1.3 mmol) in THF (5 mL) was added and the solution was allowed to warm to rt and stirred for 16 h. The solution was then quenched with water (10 mL). EtOAc (20 mL) was added and the two layers were separated, extracting the aqueous with EtOAc (3 × 20 mL). The combined organic layers were washed with water (20 mL) and brine (20 mL), dried (MgSO₄) and evaporated under reduced pressure to give the crude product. Purification by flash column chromatography on silica with petroleum ether:EtOAc (99:1) as eluent gave 2-fluoro-6-(methoxymethoxy)naphthalene as a white solid (104 mg, 38%), R_{F} 0.2 (petroleum ether:EtOAc 99:1); 50-52° C. ; IR (solid) 2956, 2936, 2909, 1603, 1579, 1510, 1376, 1360, 1226, 1152, 1078, 993, 960 cm⁻¹; ¹H NMR (400 MHz, CDCl₃) δ 7.72 - 7.66 (m, 2H, Ar), 7.40 - 7.36 (m, 2H, Ar), 7.25 - 7.18 (m, 2H, Ar), 5.27 (s, 2H, CH₂), 3.51 (s, 3H, CH₃); ¹³C NMR (101 MHz, CDCl₃) δ 159.7 (d, J= 243.5 Hz, C), 154.7 (d, J = 2.0 Hz, C), 131.5 (C), 130.1 (d, J = 9.0 Hz, C), 129.2 (d, J = 9.0 Hz, CH), 128.7 (d, J = 5.5 Hz, CH), 120.2 (CH), 116.7 (d, J = 25.0 Hz, CH), 110.8 (d, J = 20.5 Hz, CH), 110.4 (CH), 94.8 (CH₂), 56.1 (CH₃); ¹⁹F NMR (376 MHz, CDCl₃) δ -117.8 (td, J= 9.0, 5.5 Hz); LRMS (TOF MS ASAP+) m/z 206 ([M]⁺, 60), 175 ([M - OMe]⁺, 100); HRMS (TOF MS ASAP+) m/z C₁₂H₁₁O₂ F ([M]⁺) calcd for 206.0743, found 206.0742.

Next, A solution of 2-fluoro-6-(methoxymethoxy)naphthalene (103 mg, 0.5 mmol) in MeOH (5 mL) was stirred and heated at 50° C. and 6 M HCl (10 drops) was added before stirring for a further 2 h. The resulting solution was allowed to cool to rt then EtOAc (20 mL) and water (10 mL) were added and the layers were separated. The organic layer was washed with water (10 mL) and brine (10 mL), dried (MgSO₄) and evaporated under reduced pressure to give 6-fluoronaphthalen-2-ol 8o as a tan solid (77 mg, 95%); 55-57° C.; IR (solid) 3265 (O-H), 1602, 1511, 1453, 1379, 1277, 1223, 1138, 1108, 941 cm⁻¹; ¹H NMR (400 MHz, Acetone-d₆) δ 8.63 (br s, 1H, OH), 7.77 (d, J = 9.0, 1H, Ar), 7.74 (dd, J = 9.0, 2.5 Hz, 1H, Ar), 7.49 (dd, J = 9.0, 2.5 Hz, 1H, Ar), 7.26 (dd, J = 9.0, 2.5 Hz, 1H, Ar), 7.23 (dd, J = 9.0, 2.5 Hz, 1H, Ar), 7.19 (dd, J = 9.0, 2.5 Hz, 1H, Ar); ¹³C NMR (101 MHz, Acetone-d₆) δ 159.8 (d, J= 240.0 Hz, C), 155.8 (d, J= 2.5 Hz, C), 133.0 (C), 129.7 (d, J = 8.0 Hz, C), 129.6 (d, J = 5.0 Hz, CH), 129.5 (d, J = 8.5 Hz, CH), 120.5 (CH), 117.0(d, J = 25.5 Hz, CH), 111.4 (d, J = 20.5 Hz, CH), 110.1 (CH); ¹⁹F NMR (376 MHz, Acetone-d₆) δ -120.9 (td, J = 9.5, 6.0 Hz).

2-((6-Fluoronaphthalen-2-yl)oxy)acetic acid (10o). A solution of 6-fluoronaphthalen-2-ol 8o (69 mg, 0.43 mmol), ethyl bromoacetate (60 µL, 0.52 mmol) and K₂CO₃ (118 mg, 0.85 mmol) in acetone (5 mL) was stirred and heated at reflux for 16 h. The resulting solution was allowed to cool to rt then filtered and evaporated under reduced pressure. NaOH in MeOH was added to the residue and solution was stirred at rt for 3 h. The resulting solution was evaporated under reduced pressure and then acidified with 1 M HCl. Water and EtOAc was added and the two layers were separated. The aqueous layer was extracted with EtOAc (15 mL × 3). The combined organic layers were washed with brine, dried (MgSO₄) and evaporated under reduced pressure to give product 10o as an off-white solid (77 mg, 82%) 159-162° C. ; IR (solid) 2915 (O—H), 2853, 2581, 1739 (C=O) 1605, 1513, 1389, 1250, 1226, 1183, 1110, 857 cm⁻¹; ¹H NMR (400 MHz, Acetone-d₆) δ 7.89 - 7.82 (m, 2H, Ar), 7.55 (ddd, J = 10.0, 2.5, 0.5 Hz, 1H, Ar), 7.36 (d, J = 2.5 Hz, 1H, Ar), 7.34 - 7.26 (m, 2H, Ar), 4.85 (s, 2H, CH₂); ¹³C NMR (101 MHz, Acetone-d₆) δ 170.0 (C), 160.4 (d, J = 241.5 Hz, C), 156.7 (d, J= 2.0 Hz, C), 132.5 (C), 130.7 (d, J= 9.0 Hz, C), 130.2 (d, J= 9.0 Hz, CH), 129.7 (d, J= 5.0 Hz, CH), 120.7 (CH), 117.2 (d, J = 25.5 Hz, CH), 111.5 (d, J = 21.0 Hz, CH), 108.4 (CH), 65.6 (CH₂); ¹⁹F NMR (376 MHz, Acetone-d₆) δ -119.5 (td, J= 9.5, 5.5 Hz).

2-((6-Fluoronaphthalen-2-yl)oxy)-N-((2,4-dimethylphenyl)sulfonyl)acetamide (9o). A solution of naphthoxyacetic acid 10o (76 mg, 0.35 mmol), sulfonamide 11 (64 mg, 0.35 mmol), EDCI (79 mg, 0.41 mmol) and DMAP (42 mg, 0.35 mmol) in CH₂Cl₂ (5 mL) was stirred at rt for 72 h. Water was added and the two layers were separated. The organic layer was washed with 1 M HCl (×3), water, brine, dried (MgSO₄) and evaporated under reduced pressure to give the crude product. Purification by flash column chromatography on silica with petroleum ether:EtOAc (80:20) as eluent gave product 15 as a white solid (57 mg, 43%); R_{F} 0.3 (petroleum ether:EtOAc 80:20); mp 185-188° C.; IR (solid) 3269 (N—H), 1721 (C═O), 1585, 1504, 1409, 1330, 1200, 1137, 1049, 933, 860 cm⁻¹; ¹H NMR (300 MHz, Acetone-d₆) δ 10.92 (br s, 1H, NH), 7.97 (d, J= 8.0 Hz, 1H, Ar), 7.83 (d, J= 9.0 Hz, 1H, Ar), 7.75 (dd, J = 9.0, 5.5 Hz, 1H, Ar), 7.55 (dd, J= 10.0, 2.5 Hz, 1H, Ar), 7.37 - 7.23 (m, 2H, Ar), 7.23 - 7.16 (m, 2H, Ar), 7.10 (s, 1H, Ar), 4.77 (s, 2H, CH₂), 2.50 (s, 3H, CH₃), 2.35 (s, 3H, CH₃); ¹³C NMR (75.5 MHz, Acetone-d₆) δ 167.8 (C), 160.4 (d, J = 241.5 Hz, C), 156.1 (d, J = 2.5 Hz, C), 145.4 (C), 138.4 (C), 133.8 (CH), 132.2 (C), 2 × 132.0 (CH and C), 130.8 (d, J = 9.0 Hz, C), 130.2 (d, J = 9.0 Hz, CH), 129.7 (d, J = 5.5 Hz, CH), 127.4 (CH), 120.6 (CH), 117.2 (d, J = 25.5 Hz, CH), 111.5 (d, J = 20.5 Hz, CH), 108.3 (CH), 68.0 (CH₂), 21.3 (CH₃), 20.1 (CH₃); ¹⁹F NMR (282 MHz, Acetone-d₆) δ -119.1 - -119.3 (m); HRMS (ESI) calcd for C₂₀H₁₉NO₄NaS 392.0927 (M - F + Na)⁺, found 392.0915.

7-Chloronaphthalen-2-ol (8p). 7-Bromonaphthalen-2-ol (1.1 g, 5.1 mmol) in THF (10 mL) was added dropwise to a stirred suspension of NaH (1.0 g 25.4 mmol) in THF (10 mL) at rt for 30 min. MOMCl (1.0 mL, 12.7 mmol) was then added, and the solution was stirred at rt for a further 2 h. The solution was then quenched sequentially with water (10 mL) and MeOH (10 mL). Et₂O was added and the layers were separated. Aqueous layer was extracted with Et₂O (3 × 20 mL). The organic layers were combined and washed with water (20 mL), brine (20 mL), and NaHCO₃_{(aq)}, dried (MgSO₄), and evaporated under reduced pressure to give the crude product. Purification by flash column chromatography on silica with petroleum ether:EtOAc (95:5) as eluent gave 2-bromo-7-(methoxymethoxy)naphthalene as a white solid (793 mg, 58%); R_{F} 0.2 (petroleum ether:EtOAc 95:5); mp 60-63° C.; IR (solid) 2961, 2906, 1620, 1590, 1498, 1483, 1451, 1213, 1165, 1144, 1125, 1080, 991, 952, 920, 844 cm⁻¹; ¹H NMR (300 MHz, CDCl₃) δ 7.90 (d, J = 2 Hz, 1H, Ar), 7.73 (d, J = 9.0 Hz, 1H, Ar), 7.63 (d, J = 8.5 Hz, 1H), 7.42 (dd, J= 8.5, 2.0 Hz, 1H), 7.30 (d, J= 2.5 Hz, 1H), 7.22 (dd, J= 9.0, 2.5 Hz, 1H, Ar), 5.29 (s, 2H, CH₂), 3.52 (s, 3H, CH₃); ¹³C NMR (101 MHz, CDCl₃) δ 156.0 (C), 135.9 (C), 129.6 (CH), 129.4 (CH), 129.2 (CH), 128.0 (C), 127.5 (CH), 120.7 (C), 119.5 (CH), 109.4 (CH), 94.7 (CH₂), 56.3 (CH₃); LRMS (TOF MS ASAP+) m/z 266 ([M + H]⁺, 15), 235 ([M - OMe]⁺, 100), 188 ([M - Br]⁺, 5); HRMS (TOF MS ASAP+) m/z C₁₂H₁₁O₂Br ([M + H]⁺) calcd for 265.9942, found 265.9948.

Then, n-BuLi (0.8 mL of a 2.5 M solution in hexanes, 2.0 mmol) was added to a stirred solution of 2-bromo-7-(methoxymethoxy)naphthalene (350 mg, 1.3 mmol) in THF (5 mL) at -78° C. for 30 min, then a solution of NCS (170 mg, 1.3 mmol) in THF (5 mL) was added and the solution was allowed to warm to rt and stirred for 16 h. The solution was then quenched with water (10 mL). EtOAc (20 mL) was added and the two layers were separated, extracting the aqueous with EtOAc (3 × 20 mL). The combined organic layers were washed with water (20 mL) and brine (20 mL), dried (MgSO₄) and evaporated under reduced pressure to give the crude product. Purification by flash column chromatography on silica with petroleum ether:toluene (90:10) as eluent gave 2-bromo-7-(methoxymethoxy)naphthalene as a white solid (82 mg, 29%), R_{F} 0.2 (petroleum ether:toluene 90:10); 50-53° C.; IR (solid) 2902, 2829, 1623, 1589, 1498, 1407, 1252, 1218, 1153, 1073, 991, 955, 907, 881 cm⁻¹; ¹H NMR (300 MHz, CDCl₃) δ 7.75 - 7.67 (m, 3H, Ar), 7.33 - 7.27 (m, 2H, Ar), 7.22 (dd, J= 9.0, 2.5 Hz, 1H, Ar), 5.30 (s, 2H, CH₂), 3.53 (s, 3H, CH₃); ¹³C NMR (101 MHz, CDCl₃) δ 156.0 (C), 135.4 (C), 132.4 (C), 129.5 (CH), 129.3 (CH), 127.8 (C), 125.9 (CH), 125.0 (CH), 119.3 (CH), 109.4 (CH), 94.7 (CH₂), 56.3 (CH₃); LRMS (TOF MS ASAP+) m/z 222 ([M]⁺, 15), 191 ([M - OMe]⁺, 100); HRMS (TOF MS ASAP+) m/z C₁₂H₁₁O₂Cl ([M]⁺) calcd for 222.0448, found 222.0447.

Then, A solution of 2-chloro-7-(methoxymethoxy)naphthalene (82 mg, 0.37 mmol) in MeOH (5 mL) was stirred and heated at 50° C. and 6 M HCl (10 drops) was added before stirring for a further 2 h. The resulting solution was allowed to cool to rt then EtOAc (20 mL) and water (10 mL) were added and the layers were separated. The organic layer was washed with water (10 mL) and brine (10 mL), dried (MgSO₄) and evaporated under reduced pressure to give 7-chloronaphthalen-2-ol as a tan solid (56 mg, 86%); 60-63° C.; IR (solid) 3467 (O—H), 3077, 1623, 1598, 1516, 1471, 1458, 1431, 1385, 1349, 1266, 1232, 1175, 1075, 890, 838 cm⁻¹; ¹H NMR (400 MHz, CDCl₃) δ 7.72 (d, J= 9.0 Hz, 1H, Ar), 7.69 (d, J= 9.0 Hz, 1H, Ar), 7.65 (d, J= 2.0 Hz, 1H, Ar), 7.26 (dd, J= 9.0, 2.0 Hz, 1H, Ar), 7.10 (dd, J= 9.0, 2.5 Hz, 1H, Ar), 7.06 (d, J= 2.5 Hz, 1H, Ar), 5.39 (br s, 1H, OH); ¹³C NMR (101 MHz, CDCl₃) δ 154.5 (C), 135.5 (C), 132.6 (C), 129.9 (CH), 129.5 (CH), 127.3 (C), 125.2 (CH), 124.7 (CH), 118.2 (CH), 108.9 (CH). Spectroscopic data consistent with those reported in the literature.⁵⁵

2-((7-Chloronaphthalen-2-yl)oxy)acetic acid (10p). A solution of 7-chloronaphthalen-2-ol 8p (57 mg, 0.32 mmol), ethyl bromoacetate (40 µL, 0.38 mmol) and K₂CO₃ (90 mg, 0.64 mmol) in acetone (5 mL) was stirred and heated at reflux for 16 h. The resulting solution was allowed to cool to rt then filtered and evaporated under reduced pressure. NaOH in MeOH was added to the residue and solution was stirred at rt for 3 h. The resulting solution was evaporated under reduced pressure and then acidified with 1 M HCl. Water and EtOAc was added and the two layers were separated. The aqueous layer was extracted with EtOAc (×3). The combined organic layers were washed with brine, dried (MgSO₄) and evaporated under reduced pressure to give the crude product. Purification by recrystallization from toluene gave product 10p as an off-white solid (29 mg, 39%); 171-174° C. ; IR (solid) 2905 (O—H), 2584, 1717 (C═O), 1631, 1505, 1425, 1241, 1213, 1177, 1140, 1080, 1069, 835, 772 cm⁻¹; ¹H NMR (400 MHz, Acetone-d₆) δ 7.89 - 7.84 (m, 3H, Ar), 7.34 (dd, J= 9.0, 2.0 Hz, 1H, Ar), 7.31 (d, J= 2.5 Hz, 1H, Ar), 7.25 (dd, J = 9.0, 2.5 Hz, 1H, Ar), 4.86 (s, 2H, CH₂); ¹³C NMR (101 MHz, Acetone- d₆) δ 169.9 (C), 158.1 (C), 136.4 (C), 132.8 (C), 130.5 (CH), 130.4 (CH), 128.6 (C), 126.4 (CH), 125.3 (CH), 119.9 (CH), 107.5 (CH), 65.6 (CH₂); LRMS (ESI) m/z 235 ([M]⁺, 100); HRMS (ESI) m/z C₁₂H₉O₃Cl ([M]⁺) calcd for 235.0167, found 235.0169.

2-((7-Chloronaphthalen-2-yl)oxy)-N-((2,4-dimethylphenyl)sulfonyl)acetamide (9p). A solution of naphthoxyacetic acid 10p (28 mg, 0.12 mmol), sulfonamide 11 (22 mg, 0.12 mmol), EDCI (27 mg, 0.14 mmol) and DMAP (14 mg, 0.12 mmol) in CH₂Cl₂ (5 mL) was stirred at rt for 72 h. Water was added and the two layers were separated. The organic layer was washed with 1 M HCl (×3), water, brine, dried (MgSO₄) and evaporated under reduced pressure to give the crude product. Purification by flash column chromatography on silica with petroleum ether:EtOAc (80:20) as eluent gave product 11 as a white solid (10 mg, 21%); R_{F} 0.3 (petroleum ether:EtOAc 80:20); mp 175-177° C. (decomposition); IR (solid) 3269 (N-H), 2904, 1721 (C═O), 1626, 1500, 1331, 1197, 1155, 1145, 1075, 1000, 860 cm⁻¹; ¹H NMR (300 MHz, Acetone-d₆) δ 10.99 (br s, 1H, NH), 7.98 (d, J = 8.0 Hz, 1H, Ar), 7.90 - 7.82 (m, 2H, Ar), 7.70 (d, J = 2.0 Hz, 1H, Ar), 7.35 (dd, J= 8.5, 2.0 Hz, 1H, Ar), 7.27 - 7.16 (m, 2H, Ar), 7.13 -7.06 (m, 2H, Ar), 4.80 (s, 2H, CH₂), 2.48 (s, 3H, CH₃), 2.36 (s, 3H, CH₃); ¹³C NMR (75.5 MHz, Acetone-d₆) δ 167.4 (C), 157.4 (C), 145.5 (C), 138.5 (C), 136.1 (CH), 135.5 (C), 133.8 (CH), 132.7 (C), 132.1 (C), 2 × 130.5 (CH and CH), 128.6 (C), 127.4 (CH), 126.3 (CH), 125.5 (CH), 119.9 (CH), 107.4 (CH), 67.9 (CH₂), 21.3 (CH₃), 20.1 (CH₃); LRMS (TOF MS ASAP+) m/z 404 ([M+H]⁺, 100), 219 ([M -NHSO₂C₈H_{9]+}, 15); HRMS (TOF MS ASAP+) m/z C₂₀H₁₉NO₄SCl ([M+H]⁺) calcd for 404.0723, found 404.0721.

(Naphthalen-1-yl)oxyacetic acid (10q). A solution of 1-naphthol 8q (2.7 g, 25.0 mmol), ethyl bromoacetate (2.8 mL, 25.0 mmol) and K₂CO₃ (5.8 g, 41.6 mmol) in acetone (38 mL) was stirred and heated at reflux for 16 h. The resulting solution was allowed to cool to rt then filtered and evaporated under reduced pressure. NaOH in MeOH was added to the residue and solution was stirred at rt for 3 h. The resulting solution was evaporated under reduced pressure and then acidified with 1 M HCl. Water and EtOAc was added and the two layers were separated. The aqueous layer was extracted with EtOAc (30 mL × 3). The combined organic layers were washed with brine, dried (MgSO₄) and evaporated under reduced pressure to give 10q as an off-white solid (3.4 g, 67%), m.p. 193-196° C.; IR (solid) 2911, 1741, 1703, 1596, 1422, 1240, 1119 cm⁻¹; ¹H NMR (300 MHz, DMSO-d₆) δ 8.27 - 8.15 (1 H, m), 7.93 - 7.82 (1 H, m), 7.59 - 7.46 (3 H, m), 7.40 (1 H, dd, J = 8.2, 7.6), 6.88 (1 H, dd, J = 7.6, 1.0), 4.88 (2 H, s); ¹³C NMR (75.5 MHz, DMSO-d6) δ 170.0, 153.2, 134.0, 127.4, 126.5, 126.0, 125.3, 124.8, 121.6, 120.4, 105.3, 64.9.

N-((2,4-dimethylphenyl)sulfonyl)-2-(naphthalen-1-yloxy)acetamide (9q). A solution of sulfonamide 11 (183 mg, 0.99 mmol), naphthoxyacetic acid 10q (200 mg, 0.99 mmol), EDCI (228 mg, 1.19 mmol) and DMAP (121 mg, 0.99 mmol) in DCM (20 mL) was stirred for 48 hrs at rt The reaction mixture was diluted with DCM (25 mL) and washed sequentially with 10%HCl (10 mL × 3), water and brine. The organic phase was dried over MgSO₄ and solvent removed under vacuum to give the crude product. Purification by flash column chromatography on silica with 8:2 petrol:EtOAc as eluent give 9q (80 mg, 21%) as a white solid, m.p. 174-177° C.; IR (solid) 3251, 1717, 1598, 1410, 1257, 1159, 1140 cm⁻¹; ¹H NMR (300 MHz, Chloroform-d) δ 8.22 - 8.14 (1 H, m), 8.09 (1 H, d, J = 8.2), 7.90 - 7.81 (1 H, m), 7.61 - 7.51 (3 H, m), 7.31 (1 H, t, J = 8.2, 7.7), 7.20 (1 H, d, J = 8.2), 7.12 - 7.06 (1 H, m), 6.66 (1 H, dd, J = 7.7, 0.8), 4.66 (2 H, s), 2.45 (3 H, s), 2.40 (3 H, s); ¹³C NMR (75.5 MHz, CDCl₃) δ 166.4, 152.4, 145.5, 137.8, 134.8, 133.4, 133.3, 131.9, 128.1, 127.2, 127.2, 126.4, 125.6, 125.1, 122.9, 121.1, 106.0, 68.0, 21.6, 20.3.

Indol-5-yloxyacetic acid (10r). A solution of 5-hydroxyindole (2.3 g, 17.5 mmol), ethyl bromoacetate (2.3 mL, 21.03 mmol), K₂CO₃ (4.8 g, 35.0 mmol) in acetone (22 mL) was stirred and heated at reflux for 16 h. The remaining solid was filtered off, washing with acetone and the filtrate concentrated under reduced pressure. 5 M NaOH(_{aq}) (35 mL) and MeOH (17.5 mL) were added and the resulting solution was stirred at rt for 3 h. MeOH was removed under reduced pressure, and the remaining aqueous solution was acidified via addition of 6 M HCl(_{aq}). The aqueous solution was extracted using EtOAc (20 mL × 3) and the combined organic layers were washed with brine (×2), dried (MgSO₄) and evaporated under reduced pressure to give 10r as a white power, m.p. 149-157° C.; IR (solid) 3398 (O-H str), 3345 (N-H str), 2909, 2586, 1703 (C=O str), 1623, 1505, 1257 cm⁻¹; ¹H NMR (300 MHz, DMSO-d6) δ 10.90 (br s, 1H, OH), 7.26-7.28 (m, 2H, Ar), 6.98 (d, J= 2.5 Hz, 1H, Ar), 6.77 (d, J= 2.5 Hz, 1H, Ar), 6.74 (d, J= 2.5 Hz, 1H, Ar), 6.33 (s, 1H, NH), 4.61 (s, 2H, OCH₂); ¹³C NMR (75.5 MHz, DMSO-d6) δ 171.1 (C), 152.2 (C), 131.8 (C), 128.3 (C), 126.4 (CH), 112.4 (CH), 111.9 (CH), 103.3 (CH), 101.3 (CH), 65.8 (CH₂).

2-((1H-Indol-5-yl)oxy)-N-((2,4-dimethylphenyl)sulfonyl)acetamide (9r). A solution of sulfonamide 11 (174 mg, 0.94 mmol), naphthoxyacetic acid 10r (180 mg, 0.94 mmol), EDCI (216 mg, 1.13 mmol) and DMAP (115 mg, 0.94 mmol) in DCM (9.4 mL) was stirred for 48 hrs at rt The reaction mixture was diluted with DCM (20 mL) and washed sequentially with 10% HCl (5 mL × 3), water and brine. The organic phase was dried (MgSO₄) and solvent removed under vacuum to give the crude product. Purification by flash column chromatography on silica with 7:3 petrol:EtOAc as eluent give 9r (159 mg, 52%) as an orange oil, IR (film) 3417 (N-H str), 3273, 2927, 1727 (C=O str), 1601, 1583, 1480, 1222 cm⁻¹; ¹H NMR (300 MHz, CDCl₃) δ 9.04 (s, 1H, NH), 8.21 (s, 1H, NH), 8.08 (d, J= 8.0 Hz, 1H, Ar), 7.33 (d, J= 9.0 Hz, 1H, Ar), 7.24 (t, J= 3.0 Hz, 1H, Ar), 7.18 (d, J= 8.0 Hz, 1H, Ar), 7.05 (s, 1H, Ar), 6.98 (d, J= 2.5 Hz, 1H, Ar), 6.68 (dd, J = 9.0, 2.5 Hz, 1H, Ar), 6.47-6.43 (m, 1H, Ar), 4.49 (s, 2H, OCH₂), 2.47 (s, 3H, Me), 2.38 (s, 3H, Me); ¹³C NMR (75.5 MHz, CDCl₃) δ 166.9 (C), 151.1 (C), 146.2 (C), 137.6 (C), 133.3 (C), 133.2 (C), 131.9 (CH), 130.6 (CH), 128.3 (C), 127.0 (CH), 125.6 (CH), 112.2 (CH), 112.1 (CH), 104.2 (CH), 102.6 (CH), 68.3 (CH₂), 60.4 (CH₃), 40.8 (CH₃). HRMS (ESI) calcd for: C₁₈H₁₉N₂O₄S 359.1060 (M + H)⁺, found 359.1062.

1H-Indazol-5-yloxyacetic acid (10 s). A solution of 1H-inazol-5-ol (2.8 g, 20.81 mmol), ethyl bromoacetate (2.8 mL, 25.0 mmol) and K₂CO₃ (5.8 g, 41.61 mmol) in acetone (30 mL) was stirred and heated at reflux for 16 h. The remaining solids were filtered off, washing with acetone, and the filtrate was evaporated under reduced pressure. 5 M NaOH(_{aq}) (40 mL) and MeOH (20 mL) were added, and the resulting solution stirred at rt for 3 h. Then, the MeOH was removed under reduced pressure and the remaining aqueous solution acidified via addition of 6 M HCl(_{aq}). The aqueous solution was extracted with EtOAc (30 mL × 3) and the combined organic layers washed with brine, dried (MgSO₄) and evaporated under reduced pressure to give 10 s (4.1 g, 99%) as a brown solid, m.p.: stable under 300° C.; IR (solid) 3339 (O—H + N—H str), 1706 (C═O), 1611, 1511, 1102 cm⁻¹; ¹H NMR (300 MHz, DMSO-d6) δ 12.91 (br s, 2H, OH + NH), 7.93 (d, J= 1.0 Hz 1H, Ar), 7.45 (dt, J= 9.0, 1.0 Hz, 1H, Ar), 7.10 (d, J= 2.0 Hz, 1H, Ar), 7.03 (dd, J= 9.0, 2.0 Hz, 1H, Ar), 4.64 (s, 2H, OCH₂); ¹³C NMR (75.5 MHz, DMSO-d6) δ 170.5 (C), 151.8 (C), 135.9 (C), 132.6 (C), 122.8 (CH), 117.9 (CH), 111.1 (CH), 100.9 (CH), 65.3 (CH₂).

2-((1H-Indazol-5-yl)oxy)-N-((2,4-dimethylphenyl)sulfonyl)acetamide (9 s). A solution of sulfonamide 11 (335 mg, 1.81 mmol), acid 10 s (383 mg, 1.99 mmol), EDCI (385 mg, 2.01 mmol) and DMAP (242 mg, 1.98 mmol) in CH₂Cl₂ (20 mL) was stirred at rt for 48 h. CH₂Cl₂ (25 mL) was added, and the solution washed with 1 M HCl(_{aq}), water and brine then dried (MgSO₄) and evaporated under reduced pressure to give 9 s (520 mg, 88%) as a yellow solid, m.p.: stable under 300° C.; IR (solid) 3360 (N-H str), 3256, 1645 (C=O str), 1564, 1315, 1172. Compound 9 s proved to be sparingly soluble in all NMR solvents investigated. Based on spectra obtained after extended run times, we are confident that pure 9 s has indeed been prepared.

N-(Mesitylsulfonyl)-2-(naphthalen-2-ylthio)acetamide (12a). A solution of naphthalene-2-thiol (10) (32 mg, 0.2 mmol) in dry DMF (1 mL) was cooled to 0° C. with ice bath, then added NaH (8 mg, 0.2 mmol). The mixture was stirred at 0° C. for 30 min, followed by adding 7 (64 mg, 0.2 mmol), and the added mixture was stirred at room temperature overnight. After the reaction completed, it was quenched with 2 mL NH₄Cl (sat. aq.) and 10 mL water, then extracted with EtOAc (15 mL × 2). The combined EtOAc extracts were successively washed with brine, then dried with Na₂SO₄, filtered, and concentrated to the residue. This residue was further purified by preparative TLC plates (CH₂Cl₂/MeOH = 50:1) to get 12a as a white solid (61 mg, 76 %). ¹H NMR (300 MHz, Chloroform-d) δ 9.33 (s, 1H), 7.82 (dt, J= 6.6, 2.0 Hz, 1H), 7.75 (d, J= 8.7 Hz, 1H), 7.56 - 7.46 (m, 3H), 7.38 (d, J= 2.0 Hz, 1H), 7.30 - 7.25 (m, 1H), 6.63 (s, 2H), 3.72 (s, 2H), 2.39 (s, 6H), 2.19 (s, 3H). ¹³C NMR (75 MHz, Chloroform-d) δ 166.9, 143.7, 140.6, 133.7, 132.0, 131.8, 131.5, 130.6, 129.2, 127.7, 127.3, 126.8, 126.3, 125.5, 125.3, 37.1, 22.5, 21.0. HRMS (ESI) calcd for C₂₁H₂₁NO₃S₂Na 422.0861 (M +Na)⁺, found 422.0848.

N-(Mesitylsulfonyl)-2-(naphthalen-2-ylamino)acetamide (12b). Following the synthetic procedure of compound 12a, compound 12b as a white solid (31 mg, 40%). ¹H NMR (300 MHz, Chloroform-d) δ 9.37 (s, 1H), 7.72 (dd, J = 11.8, 8.4 Hz, 2H), 7.41 (dd, J= 6.0, 1.5 Hz, 2H), 7.36 - 7.29 (m, 1H), 6.91 (dd, J = 8.8, 2.5 Hz, 1H), 6.81 (s, 2H), 6.48 (d, J= 2.4 Hz, 1H), 4.53 (s, 1H), 3.88 (s, 2H), 2.43 (s, 6H), 2.32 (s, 3H). ¹³C NMR (75 MHz, Chloroform-d) δ 169.8, 143.7, 143.6, 140.6, 134.5, 132.1, 129.6, 128.6, 127.6, 126.5, 126.5, 123.4, 117.5, 105.7, 49.2, 22.5, 21.1. HRMS (ESI) calcd for C₂₁H₂₂N₂O₃SNa 405.1249 (M + Na)⁺, found 405.1233.

tert-Butyl (2-(naphthalen-2-yloxy)ethyl)carbamate (15). To a solution of naphthalene-2-ol (8a) (720 mg, 5 mmol), tert-butyl (2-hydroxyethyl)carbamate (1.2 g, 7.5 mmol) and PPh₃ (2 g, 7.5 mmol) in dry THF (10 mL) was dropwise added DEAD (1.2 g, 7.5 mmol) at 0° C. The mixture was stirred at room temperature for 12 h, added with 10 mL water and then extracted with DCM (10 mL × 3). The combined DCM extracts were successively washed with brine, then dried with Na₂SO₄, filtered, and concentrated to the residue. This residue was further purified by preparative TLC plates (hexane/EtOAc = 10:1) to yield 15 as a colorless oil (1.2 g, 81%). ¹H NMR (300 MHz, Chloroform-d) δ 7.77 (qd, J= 7.9, 7.1, 1.2 Hz, 3H), 7.46 (ddd, J= 8.2, 6.8, 1.4 Hz, 1H), 7.36 (ddd, J = 8.1, 6.8, 1.3 Hz, 1H), 7.16 (d, J= 7.9 Hz, 2H), 5.06 (s, 1H), 4.17 (t, J= 5.1 Hz, 2H), 3.63 (q, J= 5.4 Hz, 2H), 1.49 (s, 9H).

tert-Butyl 4-(naphthalen-2-yloxy)piperidine-1-carboxylate (16). Following the synthetic procedure of compound 15, compound 16 as a white solid (576 mg, 88%). ¹H NMR (300 MHz, Chloroform-d) δ 7.83 - 7.69 (m, 3H), 7.46 (ddd, J= 8.2, 6.8, 1.3 Hz, 1H), 7.36 (ddd, J= 8.1, 6.9, 1.3 Hz, 1H), 7.18 (d, J= 8.4 Hz, 2H), 4.65 (dt, J= 7.1, 3.6 Hz, 1H), 3.76 (ddd, J = 12.1, 7.5, 3.8 Hz, 2H), 3.41 (ddd, J= 13.4, 7.6, 3.9 Hz, 2H), 2.06 - 1.95 (m, 2H), 1.85 (ddt, J= 13.9, 7.4, 3.7 Hz, 2H), 1.51 (s, 9H).

2,4,6-Trimethyl-N-(2-(naphthalen-2-yloxy)ethyl)benzenesulfonamide (12c). A solution of 15 (287 mg, 1 mmol) in 5 mL dry DCM was cooled to 0° C. with ice bath. TFA (0.2 ml) was successively added to the solution at 0° C. The mixture was stirred at room temperature for 6 h and then concentrated to residue. The residue was dissolved in dry DCM (10 mL) and was cooled to 0° C. with ice bath. NEt₃ (505 mg, 5 mmol), DMAP (22 mg, 0.2 mmol), 2,4,6-trimethylbenzenesulfonyl chloride (5) (329 mg, 1.5 mmol) were successively added to the solution at 0° C. The mixture was stirred at room temperature for 12 h, added with 20 mL water and then extracted with DCM (20 mL × 3). The combined DCM extracts were successively washed with brine, then dried with Na₂SO₄, filtered, and concentrated to the residue. This residue was further purified by preparative TLC plates (hexane/EtOAc = 5:1) to 12c as a white solid (337 mg, 91%). ¹H NMR (300 MHz, Chloroform-d) δ 7.82 - 7.66 (m, 3H), 7.46 (ddd, J= 8.2, 6.7, 1.3 Hz, 1H), 7.37 (ddd, J= 8.1, 6.8, 1.3 Hz, 1H), 7.06 (dd, J= 8.9, 2.5 Hz, 1H), 6.98 (d, J= 2.5 Hz, 1H), 6.91 (s, 2H), 5.14 (t, J= 6.2 Hz, 1H), 4.04 (t, J= 5.1 Hz, 2H), 3.46 - 3.37 (m, 2H), 2.69 (s, 6H), 2.22 (s, 3H). ¹³C NMR (75 MHz, Chloroform-d) δ 155.9, 142.3, 138.9, 134.3, 133.9, 132.0, 129.5, 129.2, 127.6, 126.8, 126.5, 124.0, 118.3, 106.8, 66.0, 42.2, 22.9, 20.8. HRMS (ESI) calcd for C₂₁H₂₃NO₃SNa 392.1296 (M + Na)⁺, found 392.1281.

1-(Mesitylsulfonyl)-4-(naphthalen-2-yloxy)piperidine (12d). Following the synthetic procedure of compound 12c, compound 12d as a white solid (117 mg, 92%). ¹H NMR (300 MHz, Chloroform-d) δ 7.85 - 7.65 (m, 3H), 7.46 (ddd, J= 8.2, 6.8, 1.3 Hz, 1H), 7.36 (ddd, J= 8.1, 6.8, 1.3 Hz, 1H), 7.15 (d, J = 7.7 Hz, 2H), 6.99 (s, 2H), 4.65 (tt, J= 6.7, 3.4 Hz, 1H), 3.49 (ddd, J= 12.2, 7.4, 3.7 Hz, 2H), 3.25 (ddd, J = 12.1, 6.8, 4.0 Hz, 2H), 2.67 (s, 6H), 2.33 (s, 3H), 2.08 (ddt, J= 12.1, 7.8, 3.6 Hz, 2H), 1.96 (ddt, J= 13.5, 6.7, 3.3 Hz, 2H). ¹³C NMR (75 MHz, CDCl₃) δ 154.8, 142.5, 140.5, 134.4, 131.9, 131.9, 129.7, 129.1, 127.6, 126.7, 126.4, 123.9, 119.5, 109.0, 77.4, 77.0, 76.6, 71.1, 41.1, 29.9, 22.8, 21.0. HRMS (ESI) calcd for C₂₄H₂₇NO₃S 432.1609 (M + H)⁺, found 432.1595.

Ethyl 2-fluoro-2-(naphthalen-2-yloxy)acetate (17). To a solution of ethyl 2-bromo-2-fluoroacetate (1.8 g, 10 mmol) in dry DMF (1 mL) was added K₂CO₃ (55.2 mg, 0.4 mmol) and naphthalen-2-ol (720 mg, 5 mmol). The mixture was stirred at room temperature overnight, added with 15 mL water and then extracted with EtOAc (20 mL × 3). The combined EtOAc extracts were successively washed with brine, then dried with Na₂SO₄, filtered, and concentrated to the residue. This residue was further purified by preparative TLC plates (CH₂Cl₂/MeOH = 100: 1) to compound 17 as a white solid (496 mg, 40%). ¹H NMR (300 MHz, Chloroform-d) δ 7.95 - 7.73 (m, 3H), 7.60 - 7.41 (m, 3H), 7.33 (dd, J = 8.9, 2.5 Hz, 1H), 6.12 (d, J= 59.5 Hz, 1H), 4.41 (q, J= 7.1 Hz, 2H), 1.40 (t, J= 7.1 Hz, 3H).

2-Fluoro-2-(naphthalen-2-yloxy)acetic acid (18). A solution of lithium hydroxide (84 mg, 2.0 mmol) in water (1 mL) was added to the solution of compound 17 (248 mg, 1.0 mmol) in tetrahydrofuran (3 mL). The mixture was stirred overnight. After removal of the solvent, the residue was diluted with water (2 mL), acidified with 4 N HCl to pH 4-5, and then extracted with EtOAc (15 mL × 2). The combined organic layer was washed with brine (10 mL), dried, filtered, and then evaporated to yield compound 18 as a white solid in 76% yield. ¹H NMR (300 MHz, Chloroform-d) δ 7.95 - 7.73 (m, 3H), 7.60 - 7.41 (m, 3H), 7.33 (dd, J = 8.9, 2.5 Hz, 1H), 6.12 (d, J= 59.5 Hz, 1H), 4.41 (q, J= 7.1 Hz, 2H), 1.40 (t, J= 7.1 Hz, 3H).

2-Fluoro-N-(mesitylsulfonyl)-2-(naphthalen-2-yloxy)acetamide (12e). A solution of 5 (40 mg, 0.2 mmol) and compound 18 (44 mg, 0.2 mmol) in 2 mL dry DMF was cooled to 0° C. with ice bath. DMAP (49 mg, 0.4 mmol) and EDCI (77 mg, 0.4 mmol) were successively added to the solution at 0° C. The mixture was stirred at room temperature for 12 h, added with 10 mL water and then extracted with DCM (10 mL × 3). The combined DCM extracts were successively washed with 1N HCl and brine, then dried with Na₂SO₄, filtered, and concentrated to the residue. This residue was further purified by preparative TLC plates (CH₂Cl₂/MeOH = 50:1) to compound 20 as a white solid (69 mg, 86%). ¹H NMR (300 MHz, Chloroform-d) δ 9.05 (s, 1H), 7.96 - 7.60 (m, 3H), 7.56 - 7.38 (m, 3H), 7.24 (d, J= 9.1 Hz, 1H), 6.96 (s, 2H), 6.02 (d, J= 60.4 Hz, 1H), 2.72 (s, 6H), 2.31 (s, 3H). ¹³C NMR (75 MHz, CDCl₃) δ 152.7, 140.7, 133.8, 132.0, 130.8, 130.1, 127.7, 127.5, 126.9, 125.5, 118.4, 113.1, 22.7, 21.1. HRMS (ESI) calcd for C₂₁H₂₀FNO₄SNa 424.0995 (M + Na)⁺, found 424.0984.

2-methyl-2-(2-naphthyloxy)propanoic acid (19). A solution of 2-naphthol (2.0 g, 13.9 mmol) and NaOH (2.8 g, 69.5 mmol) in acetone (13 mL) was stirred and heated at reflux. Then, CHCl₃ (1.1 mL, 13.8 mmol) was added dropwise over 20 min. The resulting solution was stirred and heated at reflux for 4 h then evaporated under reduced pressure. H₂O (15 mL) was added and the solids were filtered off. The filtrate was acidified via addition of 6 M HCl(_{aq}) and extracted with CH₂Cl₂ (15 mL × 3). The combined organic layers were dried (MgSO₄) and evaporated under reduced pressure to give the crude product. Recrystallization from 5% EtOAc in pentane gave acid 19 (714 mg, 22%) as a tan solid, m.p. 122-126° C.; IR (solid) 2994, 1705 (C=O str), 1630, 1599, 1435, 1253, 1115 cm⁻¹; ¹H NMR (300 MHz, CDCl₃) δ 7.84 - 7.68 (m, 3H, Ar), 7.46 (ddd, J = 8.0, 7.0, 1.5, 1 H, Ar), 7.40 (ddd, J= 8.0, 7.0, 1.5, 1 H, Ar), 7.27 (d, J = 2.5 Hz, 1 H, Ar), 7.17 (dd, J= 8.5, 2.5, 1 H, Ar), 1.68 (s, 6H); ¹³C NMR (75.5 MHz, CDCl₃) δ 177.5, 152.2, 134.1, 130.3, 129.6, 127.8, 127.3, 126.6, 124.9, 122.0, 115.8, 80.2, 25.3.

N ((2,4-Dimethylphenyl)sulfonyl)-2-methyl-2-(naphthalen-2-yloxy)propenamide (12f). A solution of sulfonamide 11 (100 mg, 0.54 mmol), acid 19 (124 mg, 0.54 mmol), EDCI(124 mg, 0.65 mmol) and DMAP (66 mg, 0.54 mmol) in CH₂Cl₂ (12 mL) was stirred at rt for 48 h. Then, CH₂Cl₂ (25 mL) was added and the resulting solution was washed with 10% HCl(_{aq}) (3 × 35 mL), water (25 mL) and brine (25 mL), then dried (MgSO₄) and evaporated under reduced pressure to give the crude product. Purification by flash column chromatography on silica with 8:2 petrol:EtOAc as eluent gave product 12f (17 mg, 8%) as a colorless viscous oil, IR (film) 3253 (O-H str), 2925, 1721 (C=O str), 1598, 1465, 1341, 1177, 1099 cm⁻¹; ¹H NMR (300 MHz, CDCl₃) δ 8.09 (d, J= 8.0 Hz, 1 H, Ar), 7.82 - 7.77 (m, 1H), 7.75 (d, J= 9.0 Hz, 1H, Ar), 7.48 - 7.37 (m, 3H, Ar), 7.19 (d, J= 8.0 Hz, 1H, Ar), 7.06 (dd, J= 9.0, 2.5 Hz, 1H, Ar), 6.89 (d, J= 2.5 Hz, 1H, Ar), 6.87 (s, 1H, NH), 2.40 (s, 3H, Me), 2.21 (s, 3H, Me), 1.55 (s, 6H, CMe₂); ¹³C NMR (75.5 MHz, CDCl₃) δ 172.7, 151.4, 145.1, 137.7, 133.9, 133.4, 133.2, 131.9, 130.2, 129.9, 127.7, 127.4, 127.1, 126.5, 125.1, 121.4, 114.7, 81.4, 24.5, 21.6, 20.1.

2-(Naphthalen-2-yloxy)ethan-1-amine (20). A solution of 1-naphthol (1.0 g 6.94 mmol), 2-chloroethylamine (15.1 g, 130 mmol) and KOH (25.8 g, 460 mmol) in 3:1 PhMe:dioxane (100 mL) was stirred and heated at reflux for 18 h then cooled to rt and washed with water (5 × 60 mL). The aqueous layers were washed with EtOAc (3 × 40 mL) and the EtOAc, PhMe and dioxane layers combined, dried (MgSO₄) and evaporated under reduced pressure to give 20 (886 mg, 57%) as a brown oil, IR (film) 3055 (N-H str), 2930, 2866, 1628, 1599, 1509, 1257, 1216, 1179 cm⁻¹; ¹H NMR (300 MHz, CDC13) δ 7.81 - 7.68 (m, 3 H, Ar), 7.44 (ddd, J= 8.0, 7.0, 1.5 Hz, 1H, Ar), 7.34 (ddd, J= 8.0, 7.0, 1.5 Hz, 1H, Ar), 7.21 - 7.12 (m, 2H), 4.11 (t, J= 5.0 Hz, 2H, NCH₂), 3.15 (t, J= 5.0 Hz, 2H, ArCH₂); ¹³C NMR (75.5 MHz, CDCl₃) δ 157.0, 134.7, 129.5, 129.1, 127.8, 126.9, 126.5, 123.8, 119.0, 106.8, 70.3, 41.7.

2,4-Dimethyl-N-(2-(naphthalen-2-yloxy)ethyl)benzenesulfonamide (12 g). A solution of amine 20 (131 mg, 0.59 mmol) in CH₂Cl₂ (2.5 mL) was added dropwise to a stirred solution of 2,4-dimethylbenzenesulfonyl chloride (100 mg, 0.49 mmol) in CH₂Cl₂ (2.5 mL) at 0° C. The resulting solution was allowed to warm to rt and stirred at rt for 20 min. Then, water (15 mL) was added and the layers were separated, extracting the aqueous with CH₂Cl₂ (3 × 10 mL). The combined organic layers were dried (MgSO₄) and evaporated under reduced pressure to give the crude product. Purification by flash column chromatography on silica with 9:1 petrol:EtOAc as eluent gave 12 g (70 mg, 36%) as a yellow oil, IR (film) 3253 (N-H str), 2933, 1719, 1629, 1600, 1236, 1170, 1156 cm⁻¹; ¹H NMR (300 MHz, CDCl₃) δ 7.90 (d, J= 8.0 Hz, 1H, Ar), 7.77 (d, J= 8.0 Hz, 1H, Ar), 7.72 (d, J= 9.0 Hz, 1H, Ar), 7.67 (d, J= 8.5 Hz, 1H, Ar), 7.44 (ddd, J= 8.0, 7.0, 1.5 Hz, 2H, Ar), 7.35 (ddd, J= 8.0, 7.0, 1.5 Hz, 1H, Ar), 7.12 - 6.94 (m, 4H, Ar), 5.15 (t, J= 6.0 Hz, 1H, NH), 4.01 (t, J= 5.5 Hz, 2H, ArCH₂), 3.41 (dt, J= 5.5, 6.0 Hz, NCH₂), 2.61 (s, 3H, Me), 2.27 (s, 3H, Me); ¹³C (75.5 MHz, CDCl₃) δ 156.0, 143.7, 136.9, 135.1, 134.4, 133.4, 129.7, 129.6, 129.3, 127.8, 126.9, 126.9, 126.6, 124.1, 118.5, 106.9, 66.1, 42.5, 21.3, 20.2.

Methyl 2-(naphthalen-2-yloxy)acetate (21). Following the same synthetic procedure to compound 17. Compound 21 as a white solid (3.7 g, 87%). ¹H NMR (300 MHz, Chloroform-d) δ 7.77 (dd, J= 15.7, 8.4 Hz, 3H), 7.52 - 7.43 (m, 1H), 7.38 (ddd, J= 8.1, 6.9, 1.3 Hz, 1H), 7.28 - 7.22 (m, 1H), 7.10 (d, J= 2.5 Hz, 1H), 4.78 (s, 2H), 3.86 (s, 3H).

Methyl 2-((7-methoxynaphthalen-2-yl)oxy)acetate (22). Following the synthetic procedure to compound 17, compound 22 as a white solid (3.7 g, 87%). ¹H NMR (300 MHz, Chloroform-d) δ 7.69 (t, J= 8.1 Hz, 2H), 7.16 - 6.95 (m, 4H), 4.77 (s, 2H), 3.93 (s, 3H), 3.85 (s, 3H).

2-(Naphthalen-2-yloxy)acetic acid (23). Following the synthetic procedure to compound 18, compound 23 as a white solid (0.9 g, 88%). ¹H NMR (300 MHz, DMSO-d₆) δ 13.04 (s, 1H), 7.82 (dd, J= 14.9, 8.8 Hz, 3H), 7.46 (d, J = 1.4 Hz, 1H), 7.40 - 7.34 (m, 1H), 7.27 (d, J = 2.6 Hz, 1H), 7.24 - 7.18 (m, 1H), 4.80 (s, 2H).

2-((7-Methoxynaphthalen-2-yl)oxy)acetic acid (24). Following the synthetic procedure to compound 18, compound 24 as a white solid (2.0 g, 86%). ¹HNMR(300 MHz, DMSO-d₆) δ 13.01 (s, 1H), 7.74 (dd, J= 8.9, 3.8 Hz, 2H), 7.19 (dd, J= 13.9, 2.6 Hz, 2H), 7.00 (ddd, J= 9.2, 7.0, 2.6 Hz, 2H), 4.77 (s, 2H), 3.85 (s, 3H).

N-((3,5-Dimethylphenyl)sulfonyl)-2-(naphthalen-2-yloxy)acetamide (25a). Following the synthetic procedure to compound 12e, compound 25a as a white solid (41 mg, 57%). ¹H NMR (300 MHz, Chloroform-d) δ 9.08 (s, 1H), 7.80 (d, J= 8.6 Hz, 2H), 7.67 (d, J= 9.4 Hz, 3H), 7.52 - 7.38 (m, 2H), 7.25 (s, 1H), 7.20 (dd, J = 9.0, 2.6 Hz, 1H), 7.01 (d, J= 2.6 Hz, 1H), 4.60 (s, 2H), 2.35 (s, 6H). ¹³C NMR (75 MHz, Chloroform-d) δ 166.6, 166.2, 154.3, 139.1, 137.8, 136.0, 134.0, 130.2, 129.7, 127.7, 127.0, 126.9, 125.8, 124.7, 117.8, 107.6, 67.2, 21.2. HRMS (ESI) calcd for C₂₀H₁₉NO₄SNa 392.0932 (M + Na)⁺, found 392.0919.

N-((2-Fluorophenyl)sulfonyl)-2-(naphthalen-2-yloxy)acetamide (25b). Following the synthetic procedure to compound 12e, compound 25b as a white solid (43 mg, 60%). ¹H NMR (300 MHz, Chloroform-d) δ 8.12 (t, J= 7.5 Hz, 1H), 7.88 - 7.73 (m, 2H), 7.63 (dd, J= 15.9, 7.4 Hz, 2H), 7.44 (p, J= 7.1 Hz, 2H), 7.31 (t, J= 7.7 Hz, 1H), 7.26 - 7.18 (m, 1H), 7.04 (d, J= 8.6 Hz, 2H), 4.61 (s, 2H). ¹³C NMR (75 MHz, Chloroform-d) δ 160.7, 157.3, 154.4, 136.6, 136.4, 134.1, 131.9, 130.2, 129.7, 127.7, 127.0, 126.8, 124.7, 124.5, 124.5, 118.0, 117.2, 116.9, 107.6, 67.3. HRMS (ESI) calcd for C₁₈H₁₄FNO₄SNa 382.0525 (M + Na)⁺, found 382.0511.

N-((3-Fluorophenyl)sulfonyl)-2-(naphthalen-2-yloxy)acetamide (25c). Following the synthetic procedure to compound 12e, compound 25c as a white solid (46 mg, 64%). ¹H NMR (300 MHz, Chloroform-d) δ 9.15 (s, 1H), 7.90 - 7.85 (m, 1H), 7.81 (dd, J= 9.4, 3.3 Hz, 3H), 7.68 (d, J= 8.1 Hz, 1H), 7.53 - 7.39 (m, 3H), 7.35 (dt, J = 8.3, 4.2 Hz, 1H), 7.19 (dd, J = 9.0, 2.6 Hz, 1H), 7.03 (d, J = 2.6 Hz, 1H), 4.61 (s, 2H). ¹³C NMR (75 MHz, Chloroform-d) δ 166.2, 163.8, 160.4, 154.2, 139.9 (d, J = 7.3 Hz), 134.0, 130.8, 130.7, 130.3, 129.8, 127.7, 127.0, 126.9, 124.8, 124.3, 124.2, 121.7, 121.4, 117.7, 116.1, 115.7, 107.7, 67.2. HRMS (ESI) calcd for C₁₈H₁₄FNO₄SNa 382.0525 (M + Na)⁺, found 382.0511.

N-((4-Fluorophenyl)sulfonyl)-2-(naphthalen-2-yloxy)acetamide (25d). Following the synthetic procedure to compound 12e, compound 25d as a white solid (44 mg, 61%). ¹H NMR (300 MHz, Chloroform-d) δ 9.24 (s, 1H), 8.12 - 8.04 (m, 2H), 7.79 (dd, J= 8.6, 4.1 Hz, 2H), 7.65 (d, J= 8.1 Hz, 1H), 7.51 - 7.38 (m, 2H), 7.21 - 7.10 (m, 3H), 6.99 (d, J= 2.6 Hz, 1H), 4.59 (s, 2H). ¹³C NMR (75 MHz, Chloroform-d) δ 167.7, 166.5, 164.3, 154.2, 134.0, 133.9, 131.6, 131.4, 130.2, 129.7, 127.7, 127.0, 126.9, 124.8, 117.8, 116.5, 116.2, 107.6, 67.2. HRMS (ESI) calcd for C₁₈H₁₄FNO₄SNa 382.0525 (M + Na)⁺, found 382.0511.

N-((3-Fluoro-4-nitrophenyl)sulfonyl)-2-(naphthalen-2-yloxy)acetamide (25e). Following the synthetic procedure to compound 12e, compound 25e as a yellow solid (113 mg, 56%). ¹H NMR (300 MHz, DMSO-d6) δ 8.11 (d, J= 9.1 Hz, 1H), 7.82 (d, J= 8.6 Hz, 2H), 7.78 - 7.60 (m, 4H), 7.39 (dt, J = 23.9, 7.1 Hz, 2H), 7.16 (dd, J= 9.0, 2.6 Hz, 1H), 7.09 - 6.96 (m, 2H), 4.80 (s, 2H). ¹³C NMR (75 MHz, DMSO) δ 168.1, 155.7, 145.9, 145.2, 134.2, 132.5, 129.9, 129.2, 127.9, 127.7, 127.0, 126.9, 124.4, 119.8, 118.8, 112.4, 107.4, 66.6. HRMS (ESI) calcd for C₁₈H₁₄FN₂O₆S 405.0557 (M + H)⁺, found 4050537.

N-((4-Amino-3-fluorophenyl)sulfonyl)-2-(naphthalen-2-yloxy)acetamide (25f). A solution of 25e (100 mg, 0.27 mmol) in EtOH (10 mL) was treated with Pd/C (10 mg) and purged with H₂ for 10 min. A slightly positive pressure of H₂ was introduced into the flask and the reaction mixture was heated at 60° C. with vigorous stirring for 3 hours. After cooling to room temperature, the reaction mixture was filtered through a pad of Celite, and the pad was washed with ethyl acetate. The combined filtrates were concentrated in vacuo and the residue was purified by silica gel chromatography (Eluent: 5% MeOH in DCM) to provide the product as a yellow solid (93 mg, 92%). ¹H NMR (300 MHz, DMSO-d₆) δ 7.97 (d, J= 9.0 Hz, 1H), 7.79 (dd, J= 8.5, 4.1 Hz, 2H), 7.70 - 7.51 (m, 4H), 7.36 (dt, J = 28.5, 7.2 Hz, 2H), 7.12 (dd, J= 8.9, 2.5 Hz, 1H), 7.05 - 6.90 (m, 2H), 4.54 (s, 2H), 3.57 (s, 2H). ¹³C NMR (75 MHz, DMSO) δ 171.4, 166.6, 156.5, 150.4, 146.1, 134.5, 131.2, 129.5, 128.9, 127.9, 127.0, 126.7, 126.3, 124.0, 119.1, 118.6, 113.4, 107.3, 68.4. HRMS (ESI) calcd for C₁₈H₁₆FN₂O₄S 375.0815 (M + H)⁺, found 375.0819.

N-((2,4-Dimethoxyphenyl)sulfonyl)-2-(naphthalen-2-yloxy)acetamide (25 g). Following the synthetic procedure to compound 12e, compound 25 g as a white solid (31 mg, 39%). ¹H NMR (300 MHz, Chloroform-d) δ 9.11 (s, 1H), 8.06 (d, J = 8.9 Hz, 1H), 7.82 (d, J= 8.6 Hz, 2H), 7.63 (d, J= 8.1 Hz, 1H), 7.45 (dt, J= 19.5, 7.2 Hz, 2H), 7.21 (dd, J= 9.0, 2.6 Hz, 1H), 6.98 (d, J= 2.6 Hz, 1H), 6.60 (dd, J= 9.0, 2.2 Hz, 1H), 6.26 (d, J= 2.3 Hz, 1H), 4.61 (s, 2H), 3.86 (s, 3H), 3.47 (s, 3H). ¹³C NMR (75 MHz, Chloroform-d) δ 166.6, 166.0, 158.3, 154.7, 134.1, 133.8, 130.1, 129. 7, 127.7, 127.0, 126.9, 124.7, 118.0, 107.4, 104.6, 99.2, 67.6, 55.9, 55.8. HRMS (ESI) calcd for C₂₀H₁₉NO₆SNa 424.0831 (M + Na)⁺, found 424.0816.

N-((2,5-Dimethoxyphenyl)sulfonyl)-2-(naphthalen-2-yloxy)acetamide (25h). Following the synthetic procedure to compound 12e, compound 25h as a white solid (64 mg, 80%). ¹H NMR (300 MHz, Chloroform-d) δ 9.17 (s, 1H), 7.83 (d, J= 9.2 Hz, 2H), 7.69 - 7.60 (m, 2H), 7.46 (dt, J= 19.7, 7.1 Hz, 2H), 7.21 (dd, J= 9.0, 2.6 Hz, 1H), 7.11 (dd, J= 9.0, 3.2 Hz, 1H), 7.00 (d, J= 2.6 Hz, 1H), 6.76 (d, J= 9.1 Hz, 1H), 4.64 (s, 2H), 3.86 (s, 3H), 3.51 (s, 3H). ¹³C NMR (75 MHz, CDCl₃) δ 166.6, 154.6, 153.0, 134.1, 130.2, 129.7, 127.7, 127.0, 126.9, 124.7, 122.6, 117. 9, 115.2, 113.6, 107.5, 67.6, 56.4, 56.1. HRMS (ESI) calcd for C₂₀H₂₀NO₆S 402.1011 (M + H)⁺, found 402.0999.

N-((3,4-Dimethoxyphenyl)sulfonyl)-2-(naphthalen-2-yloxy)acetamide (25i). Following the synthetic procedure to compound 12e, compound 25i as a white solid (60 mg, 75%). ¹H NMR (300 MHz, Chloroform-d) δ 9.00 (s, 1H), 7.81 (d, J= 8.5 Hz, 2H), 7.73 - 7.61 (m, 2H), 7.52 (d, J= 2.2 Hz, 1H), 7.45 (dt, J= 20.0, 7.0 Hz, 2H), 7.19 (dd, J= 9.0, 2.6 Hz, 1H), 6.99 (d, J= 2.6 Hz, 1H), 6.90 (d, J= 8.6 Hz, 1H), 4.60 (s, 2H), 3.95 (s, 3H), 3.87 (s, 3H). 13C NMR (75 MHz, CDCl₃) δ 166.6, 154.3, 153.9, 148.9, 134.0, 130.2, 129.7, 127.7, 127.0, 126.9, 124.7, 122.9, 117.8, 110.7, 110.3, 107.6, 67.3, 56.2 (2C). HRMS (ESI) calcd for C₂₀H₂₀NO₆S 402.1011 (M + H)⁺, found 402.0999.

N-((3-Bromo-5-methylphenyl)sulfonyl)-2-(naphthalen-2-yloxy)acetamide (25j). Following the synthetic procedure to compound 12e, compound 25j as a white solid (69 mg, 75%). ¹H NMR (300 MHz, Chloroform-d) δ 9.06 (s, 1H), 8.03 (d, J= 1.9 Hz, 1H), 7.82 (d, J= 8.6 Hz, 3H), 7.68 (d, J= 8.0 Hz, 1H), 7.57 (s, 1H), 7.45 (dt, J= 20.6, 7.1 Hz, 2H), 7.20 (dd, J= 9.0, 2.7 Hz, 1H), 7.03 (d, J= 2.7 Hz, 1H), 4.61 (s, 2H), 2.37 (s, 3H). ¹³C NMR (75 MHz, CDCl₃) δ 154.2, 141.4, 137.9, 134.0, 130.3, 129.7, 128.2, 127.7, 127.5, 127.0, 126.9, 124.8, 122.6, 117.7, 107.7, 67.2, 21.1. HRMS (ESI) calcd for C₁₉H₁₆BrNO₄SNa 455.9876 (M + Na)⁺, found 455.9872.

N-((3-Bromo-5-methylphenyl)sulfonyl)-2-((7-methoxynaphthalen-2-yl)oxy)acetamide (25k). Following the synthetic procedure to compound 12e, compound 25k as a white solid (61 mg, 66%). ¹H NMR (300 MHz, Chloroform-d) δ 9.09 (s, 1H), 8.02 (s, 1H), 7.79 (s, 1H), 7.69 (t, J= 9.0 Hz, 2H), 7.56 (s, 1H), 7.12 - 6.89 (m, 4H), 4.59 (s, 2H), 3.91 (s, 3H), 2.35 (s, 3H). ¹³C NMR (75 MHz, CDCl₃) δ 158.5, 154.8, 141.4, 137.8, 135.5, 129.9, 129.2, 128.2, 127.5, 125.1, 122.5, 117.3, 115.0, 107.0, 105.4, 67.3, 55.3, 21.0. HRMS (ESI) calcd for C₂₀H₁₈BrNO₅SNa 485.9987 (M + Na)⁺, found 485.9985.

N-((3,5-Dichlorophenyl)sulfonyl)-2-((7-methoxynaphthalen-2-yl)oxy)acetamide (251). Following the synthetic procedure to compound 12e, compound 251 as a white solid (67 mg, 76%). ¹H NMR (300 MHz, Chloroform-d) δ 9.11 (s, 1H), 7.96 (d, J= 1.9 Hz, 2H), 7.72 (dd, J= 11.2, 8.9 Hz, 2H), 7.59 (t, J= 1.9 Hz, 1H), 7.16 - 6.90 (m, 4H), 4.63 (s, 2H), 3.93 (s, 3H). ¹³C NMR (75 MHz, CDCl₃) δ 166.6, 158.6, 154.7, 136.0, 135.5, 134.2, 130.1, 129.3, 126.8, 125.1, 117.5, 114.9, 107.1, 105.3, 67.2, 55.3. HRMS (ESI) calcd for C₁₉H₁₆Cl₂NO₅S 440.0126 (M + H)⁺, found 440.0117.

N-((2-Methoxy-4-nitrophenyl)sulfonyl)-2-((7-methoxynaphthalen-2-yl)oxy)acetamide (25 m). Following the synthetic procedure to compound 12e, compound 25 m as a white solid (52 mg, 58%). ¹H NMR (300 MHz, DMSO-d6) δ 12.82 (s, 1H), 8.12 (d, J= 9.3 Hz, 1H), 8.00 - 7.87 (m, 2H), 7.73 (dd, J= 8.9, 2.2 Hz, 2H), 7.10 (d, J= 2.5 Hz, 1H), 7.06 - 6.88 (m, 3H), 4.78 (s, 2H), 4.05 (s, 3H), 3.85 (s, 3H). ¹³C NMR (75 MHz, DMSO) δ 168.0, 158.3, 157.6, 156.4, 152.2, 135.8, 132.8, 132.3, 129.6, 129.5, 124.5, 116.8, 115.8, 115.4, 108.7, 107.0, 105.7, 66.5, 57.8, 55.6. HRMS (ESI) calcd for C₂₀H₁₉N₂O₈S 447.0862 (M + H)⁺, found 447.0857.

N-((4-Amino-2-methoxyphenyl)sulfonyl)-2-((7-methoxynaphthalen-2-yl)oxy)acetamide (25n). Following the synthetic procedure to compound 25f, compound 25n as a white solid (87 mg, 94%). ¹H NMR (300 MHz, DMSO-d₆) δ 11.90 (s, 1H), 7.71 (d, J= 8.7 Hz, 2H), 7.44 (d, J= 8.8 Hz, 1H), 7.20 - 6.84 (m, 4H), 6.35 - 6.00 (m, 4H), 4.69 (s, 2H), 3.82 (d, J= 19.3 Hz, 5H). ¹³C NMR (75 MHz, DMSO) δ 167.0, 159.0, 158.3, 156.6, 156.3, 135.8, 133.1, 129.6, 129.5, 124.5, 116.7, 115.9, 112.2, 106.9, 105.6, 105.1, 96.5, 66.4, 56.1, 55.6. HRMS (ESI) calcd for C₂₀H₂₁N₂O₆S 417.1120 (M + H)⁺, found 417.1113.

N-((2,5-Dimethoxyphenyl)sulfonyl)-2-((7-methoxynaphthalen-2-yl)oxy)acetamide (25o). Following the synthetic procedure to compound 12e, compound 25o as a white solid (61 mg, 71%). ¹H NMR (300 MHz, Chloroform-d) δ 9.09 (s, 1H), 7.70 (t, J= 9.1 Hz, 2H), 7.62 (d, J= 2.9 Hz, 1H), 7.06 (qd, J= 9.0, 3.0 Hz, 3H), 6.91 (d, J= 10.8 Hz, 2H), 6.73 (d, J= 9.0 Hz, 1H), 4.60 (s, 2H), 3.90 (s, 3H), 3.84 (s, 3H), 3.49 (s, 3H). ¹³C NMR (75 MHz, CDCl₃) δ 158.5, 155.3, 153.0, 150.7, 135.6, 129.8, 129.1, 125.0, 122.4, 117.3, 115.2, 115.1, 113.6, 106.8, 105.3, 67.6, 56.3, 56.1, 55.3. HRMS (ESI) calcd for C₂₁H₂₂NO₇S 432.1117 (M + H)⁺, found 432.1116.

N-((3,4-Dimethoxyphenyl)sulfonyl)-2-((7-methoxynaphthalen-2-yl)oxy)acetamide (25p). Following the synthetic procedure to compound 12e, compound 25p as a white solid (53 mg, 62%). ¹H NMR (300 MHz, Chloroform-d) δ 8.99 (s, 1H), 7.77 - 7.59 (m, 3H), 7.51 (d, J = 2.3 Hz, 1H), 7.05 (ddd, J= 14.7, 8.8, 2.5 Hz, 2H), 6.97 - 6.84 (m, 3H), 4.59 (s, 2H), 3.94 (s, 3H), 3.91 (s, 3H), 3.87 (s, 3H). ¹³C NMR (75 MHz, CDCl₃) δ 166.6, 158.6, 154.9, 148.9, 135.5, 129.9, 129.2, 125.0, 122.9, 117.4, 115.1, 110.7, 110.3, 106.9, 105.3, 67.3, 56.2, 55.3. HRMS (ESI) calcd for C₂₁H₂₂NO₇S 402.1011 (M + H)⁺, found 402.0999.

2-((7-Methoxynaphthalen-2-yl)oxy)-N-(naphthalen-2-ylsulfonyl)acetamide (25q). Following the same synthetic procedure to compound 12e, compound 25q as a white solid (54 mg, 64%). ¹H NMR (300 MHz, Chloroform-d) δ 9.05 (s, 1H), 8.75 - 8.62 (m, 1H), 7.97 (d, J = 8.0 Hz, 1H), 7.93 -7.80 (m, 3H), 7.76 - 7.58 (m, 4H), 7.05 (td, J= 9.3, 2.5 Hz, 2H), 6.87 (dd, J= 9.1, 2.5 Hz, 2H), 4.58 (s, 2H), 3.83 (s, 3H). ¹³C NMR (75 MHz, CDCl₃) δ 166.3, 158.5, 154.8, 135.5, 134.7, 131.8, 130.8, 130.0, 129.7, 129.5, 129.2, 129.2, 127.9, 127.7, 125.1, 122.6, 117.4, 115.1, 106.9, 105.3, 67.3, 55.2. HRMS (ESI) calcd for C₂₃H₂₀NO₅S 422.1062 (M + H)⁺, found 422.1053.

N-((3-(5-Fluoropyridin-3-yl)-5-methylphenyl)sulfonyl)-2-(naphthalen-2-yloxy)acetamide (25r). To a stirred solution of compound 25j (87 mg, 0.2 mmol) in 1,4-dioxane (2 mL) was added (5-fluoropyridin-3-yl)boronic acid (28 mg, 0.2 mmol), K₂CO₃ (55.2 mg, 0.4 mmol) and [1,1′-bis(diphenylphosphino)ferrocene]dichloropalladium(II) (8 mg, 0.01 mmol), and the reaction mixture was heated at 110° C. for overnight. After cooling to room temperature, the reaction mixture was filtered through a pad of Celite, and the pad was washed with ethyl acetate. The combined filtrates were concentrated in vacuo and the residue was purified by silica gel chromatography (Eluent: 2% ethyl acetate in petroleum ether) to provide the product 25r (62 mg, 71%) as a white solid. ¹H NMR (300 MHz, DMSO-d₆) δ 12.48 (s, 1H), 8.76 (s, 1H), 8.64 (s, 1H), 8.04 (s, 2H), 7.93 (s, 1H), 7.84 - 7.74 (m, 3H), 7.58 (d, J= 8.0 Hz, 1H), 7.36 (p, J= 7.2 Hz, 2H), 7.15 (d, J = 8.4 Hz, 1H), 7.06 (s, 1H), 4.81 (s, 2H), 2.45 (s, 3H). ¹³C NMR (75 MHz, DMSO) δ 168.2, 155.8, 144.5, 144.4, 141.0, 140.5, 137.9, 137.6, 136.9, 136.4, 134.3, 133.6, 129.8, 129.1, 128.0, 127.9, 127.0, 126.8, 124.3, 123.4, 121.9, 121.7, 118.8, 107.4, 66.7, 21.3. HRMS (ESI) calcd for C₂₄H₂₀FN₂O₄S 451.1128 (M + H)⁺, found 451.1120.

N-((3-(Furan-2-yl)-5-methylphenyl)sulfonyl)-2-(naphthalen-2-yloxy)acetamide (25 s). Following the synthetic procedure to compound 25r, compound 25 s as a white solid (33 mg, 79%). ¹H NMR (300 MHz, Chloroform-d) δ 9.02 (s, 1H), 8.15 (s, 1H), 7.86 - 7.69 (m, 4H), 7.64 (d, J = 7.9 Hz, 1H), 7.53 -7.36 (m, 3H), 7.19 (dd, J = 9.0, 2.6 Hz, 1H), 7.01 (d, J= 2.6 Hz, 1H), 6.75 (d, J = 3.4 Hz, 1H), 6.50 (dd, J= 3.4, 1.8 Hz, 1H), 4.61 (s, 2H), 2.42 (s, 3H). ¹³C NMR (75 MHz, CDCl₃) δ 166.1, 154.2, 151.9, 143.0, 139.8, 138.6, 134.0, 131.8, 130.2, 129.7, 127.7, 127.1, 127.0, 126.9, 124.7, 120.8, 117.7, 111.9, 107.7, 106.9, 67.3, 21.4. HRMS (ESI) calcd for C₂₃H₂₀NO₅S 422.1062 (M + H)⁺, found 422.1056.

N-((3-Methyl-5-(1-methyl-1H-pyrazol-5-yl)phenyl)sulfonyl)-2-(naphthalen-2-yloxy)acetamide (25t). Following the synthetic procedure to compound 25r, compound 25t as a white solid (27 mg, 63%). ¹H NMR (300 MHz, Chloroform-d) δ 7.93 (d, J= 28.3 Hz, 2H), 7.75 (s, 2H), 7.50 (q, J= 20.7 Hz, 5H), 7.19 (d, J= 8.8 Hz, 1H), 7.00 (s, 1H), 6.35 (s, 1H), 4.57 (s, 2H), 3.88 (s, 3H), 2.45 (s, 3H). ¹³C NMR (75 MHz, CDCl₃) δ 154.4, 139.9, 138.7, 134.0, 131.6, 130.1, 129.6, 127.6, 126.9, 126.8, 124.6, 117.7, 107.8, 106.7, 37.6, 21.3. HRMS (ESI) calcd for C₂₃H₂₂N₃O₄S 436.1331 (M + H)⁺, found 436.1323.

N-((3-Methyl-5-((3-(trifluoromethyl)pyridin-2-yl)amino)phenyl)sulfonyl)-2-(naphthalen-2-yloxy)acetamide (25u). To a stirred solution of compound 25j (87 mg, 0.2 mmol) in 1,4-dioxane (2 mL) was added 3-(trifluoromethyl)pyridin-2-amine (32.4 mg, 0.2 mmol), Cs₂CO₃ (130 mg, 0.4 mmol), XantPhos (11.6 mg, 0.02) and Pd(OAc)₂ (2 mg, 0.01 mmol), and the reaction mixture was heated at 100° C. for overnight. After cooling to room temperature, the reaction mixture was filtered through a pad of Celite, and the pad was washed with ethyl acetate. The combined filtrates were concentrated in vacuo and the residue was purified by silica gel chromatography (Eluent: 50% ethyl acetate in petroleum ether) to provide the product 25u (52 g, 50%) as a white solid. ¹H NMR (300 MHz, DMSO-d₆) δ 12.43 (s, 1H), 8.42 (d, J= 25.1 Hz, 2H), 8.03 (d, J = 9.9 Hz, 2H), 7.80 (d, J = 8.4 Hz, 2H), 7.66 (d, J = 8.7 Hz, 2H), 7.36 (d, J = 15.0 Hz, 3H), 7.22 - 6.90 (m, 3H), 4.80 (s, 2H), 2.32 (s, 3H). ¹³C NMR (75 MHz, DMSO-d₆) δ 167.6, 166.6, 155.8, 152.0, 151.9, 141.3, 139.8, 139.1, 136.8 (d, J= 5.3 Hz), 134.3, 129.8, 129.2, 127.9, 127.6, 127.1, 126.9, 126.0, 124.4, 122.4, 121.9, 118.7, 117.9, 115.9, 110.6 (dd, J = 62.6, 31.3 Hz), 107.5, 66.6, 21.4. HRMS (ESI) calcd for C₂₅H₂₁F₃N₃O₄S 516.1205 (M + H)⁺, found 516.1199.

N-((3-Methyl-5-((5-(trifluoromethyl)pyridin-2-yl)amino)phenyl)sulfonyl)-2-(naphthalen-2-yloxy)acetamide (25v). Following the synthetic procedure to compound 25u, compound 25v as a white solid (37 mg, 72%). ¹H NMR (300 MHz, Chloroform-d) δ 8.50 (s, 1H), 7.97 (s, 1H), 7.75 (d, J= 20.6 Hz, 3H), 7.65 (d, J= 9.3 Hz, 2H), 7.56 (s, 1H), 7.49 - 7.38 (m, 2H), 7.19 (d, J = 8.9 Hz, 1H), 7.01 (s, 1H), 6.84 (d, J = 9.6 Hz, 2H), 4.62 (s, 2H), 2.40 (s, 3H). ¹³C NMR (75 MHz, CDCl₃) δ 154.2, 154.2, 140.6 (2C), 140.1, 134.9, 134.0, 130.3, 129.7, 127.7 (2C), 126.9, 126.1, 124.8, 123.0, 117.7, 116.7, 108.8, 107.7, 67.3, 21.5. HRMS (ESI) calcd for C₂₅H₂₁F₃N₃O₄S 516.1205 (M + H)⁺, found 516.1198.

2-(Naphthalen-2-yloxy)-N-tosylacetamide (25w). A solution of acid 23 (200 mg, 0.99 mmol), p-toluenesulfonamide (170 mg, 0.99 mmol), EDCI (228 mg, 1.19 mmol) and DMAP (121 mg, 0.99 mmol) in CH₂Cl₂ (15 mL) was stirred at rt for 44 h. Then, CH₂Cl₂ (25 mL) was added and the resulting solution was washed with 10% HCl_{(aq)} (3 × 25 mL), water (25 mL) and brine (25 mL), dried (MgSO₄) and evaporated under reduced pressure to give the crude product. Recrystallisation from MeOH gave 25w (205 mg, 58%) as a white solid, m.p. 165-168° C.; IR (solid) 3310 (N-H str), 1720 (C=O str), 1629, 1418, 1201, 1179, 1159 cm⁻¹; ¹H NMR (300 MHz, CDCl₃) δ 8.91 (s, 1H, NH), 7.93 (dt, J = 8.5, 2.0 Hz, 2H, Ar), 7.85 - 7.77 (m, 2H, Ar), 7.65 (d, J = 8.0, 1H, Ar), 7.48 (ddd, J = 8.0, 7.0, 1.5 Hz, 1H, Ar), 7.41 (ddd, J = 8.0, 7.0, 1.5 Hz, 1H, Ar), 7.29 (d, J = 8.0 Hz, 2H, Ar), 7.18 (dd, J = 9.0, 2.5 Hz, 2H, Ar), 6.99 (d, J = 2.5 Hz, 1H, Ar), 4.58 (s, 2H, OCH₂), 2.43 (s, 3H, Me); ¹³C NMR (75.5 MHz, CDCl₃) δ 166.2, 154.4, 145.6, 135.2, 134.2, 130.4, 129.9, 129.8, 128.7, 127.9, 127.2, 127.1, 124.9, 117.9, 107.8, 67.4, 21.9.

3-Nitrobenzenesulfonamide (26). 35% NH₄OH_{(aq)} (3 mL) was added dropwise to a stirred solution of 3-nitrobenzenesulfonyl chloride (1.0 g, 4.52 mmol) in THF (3 mL) at 0° C. The resulting solution was allowed to warm to rt and stirred at rt for 18 h. Water (10 mL) was added and the resulting solution was extracted with EtOAc (3 × 30 mL). The combined organic layers were dried (Na₂SO₄) and evaporated under reduced pressure to give 26 (875 mg, 96%) as a white solid, m.p. 164-167° C.; IR (solid) 3341 (N-H str), 3261 (N-H str), 3095, 1606, 1529, 1333, 1184 cm⁻¹; ¹H NMR (300 MHz, DMSO-d₆) δ 8.60 (t, J = 2.0 Hz, 1H, Ar), 8.45 (ddd, J = 8.0, 2.0, 1.0 Hz, 1H, Ar), 8.24 (ddd, J = 8.0, 2.0, 1.0 Hz, 1H, Ar), 7.89 (t, J = 8.0 Hz, 1H, Ar), 7.71 (br s, 2H, NH₂); ¹³C NMR (75.5 MHz, DMSO-d₆) δ 147.7, 145.6, 131.7, 131.1, 126.5, 120.5.

2-(Naphthalen-2-yloxy)-N-((3-nitrophenyl)sulfonyl)acetamide (25x). A solution of acid 23 (200 mg, 0.99 mmol), sulfonamide 26 (200 mg, 0.99 mmol), EDCI (228 mg, 1.19 mmol) and DMAP (121 g, 0.99 mmol) in CH₂Cl₂ (10 mL) was stirred at rt for 42 h. Then, CH₂Cl₂ (25 mL) was added and the resulting solution was washed with 10% HCl_{(aq)} (3 × 25 mL), water (25 mL) and brine (25 mL) then dried (MgSO₄) and evaporated under reduced pressure to give the crude product. Purification by flash column chromatography on silica with 8:2 CH₂Cl_{2:}MeOH gave 25x (167 mg, 44%) as a yellow solid, m.p. 145-149° C.; IR (solid) 3566 (N-H str), 3522, 3359, 1631, 1597, 1532, 1390, 1352, 1174, 1117; ¹H NMR (300 MHz, DMSO-d₆) δ 8.54 (t, J = 2.0 Hz, 1H, Ar), 8.29 (ddd, J = 8.0, 2.5, 1.0 Hz, 1H, Ar), 8.20 (dt, J = 8.0, 1.5 Hz, 1H, Ar), 7.83 - 7.66 (m, 3H, Ar), 7.61 (d, J = 8.0 Hz, 1H, Ar), 7.40 (ddd, J = 8.0, 7.0, 1.5 Hz, 1H, Ar), 7.31 (ddd, J = 8.0, 7.0, 1.5 Hz, 1H, Ar), 7.09 (dd, J = 9.0, 2.5 Hz, 1H, Ar), 7.00 (d, J = 2.5 Hz, 1H, Ar), 4.48 (s, 2H, OCH₂); ¹³C (75.5 MHz, DMSO-d₆) δ 171.7, 156.2, 147.2, 146.1, 134.1, 133.3, 130.1, 129.2, 128.5, 127.6, 126.6, 126.4, 125.7, 123.7, 121.9, 118.7, 107.0, 68.2.

Benzenesulfonamide (27). 35% NH₄OH_{(aq)} (3 mL) was added dropwise to a stirred solution of benzenesulfonyl chloride (1.0 g, 5.66 mmol) in THF (3 mL) at 0° C. The resulting solution was allowed to warm to rt and stirred at rt for 23 h. Then, water (15 mL) was added and the resulting solution was extracted with EtOAc (3 × 40 mL). The combined organic layers were dried (MgSO₄) and evaporated under reduced pressure to give 27 (476 mg, 54%) as a white solid, m.p. 150-152° C.; IR (solid) 3346 (N-H str), 3253 (N-H str), 1447, 1331, 1310, 1180, 1154 cm⁻¹; ¹H NMR (300 MHz, DMSO-d₆) δ 7.89 - 7.75 (m, 2H, Ar), 7.65 - 7.51 (m, 3H, Ar), 7.34 (s, 2H, NH₂); ¹³C NMR (75.5 MHz, DMSO-d₆) δ 144.1, 131.8, 128.9, 125.5.

2-(Naphthalen-2-yloxy)-N-(phenylsulfonyl)acetamide (25y). A solution of benzenesulfonamide 27 (455 mg, 0.99 mmol), acid 23 (200 mg, 0.99 mmol), EDCI (228 mg, 1.19 mmol) and DMAP (121 mg, 0.99 mmol) in CH₂Cl₂ (10 mL) was stirred at rt for 26 h. Then, CH₂Cl₂ (25 mL) was added and the resulting solution was washed with 10% HCl_{(aq)} (3 × 25 mL), water (25 mL) and brine (25 mL ), dried (MgSO₄) and evaporated under reduced pressure to give the crude product. Recrystallisation from MeOH gave 25y (146 mg, 43%) as a white solid, m.p. 174-178° C.; IR (solid) 3312 (N-H str), 1724 (C=O str), 1627, 1602, 1418, 1370, 1187, 1160 cm⁻¹; ¹H NMR (300 MHz, CDCl₃) δ 8.95 (s, 1H, NH), 8.11 - 8.02 (m, 2H, Ar), 7.81 (dd, J = 9.0, 2.5 Hz, 2H, Ar), 7.70 - 7.60 (m, 2H, Ar), 7.57 - 7.36 (m, 4H, Ar), 7.18 (dd, J = 9.0, 2.5 Hz, 1H, Ar), 7.01 (d, J = 2.5 Hz 1H, Ar), 4.59 (s, 2H, OCH₂); ¹³C NMR (75.5 MHz, CDCl₃) δ 166.2, 154.3, 138.2, 134.4, 134.2, 130.5, 129.9, 129.2, 128.7, 127.9, 127.2, 127.1, 125.0, 117.9, 107.8, 67.4.

3-Methoxybenzenesulfonamide (28). 35% NH₄OH_{(aq)} (3 mL) was added dropwise to a stirred solution of 3-methoxybenzenesulfonamide (0.5 g, 2.42 mmol) in THF (3 mL) at 0° C. The resulting solution was allowed to warm to rt and stirred at rt for 26 h. Then, water (20 mL) was added and the resulting solution was extracted with EtOAc (4 × 20 mL). The combined organic layers were dried (MgSO₄) and evaporated under reduced pressure to give 28 (413 mg, 91%) as an off-white solid, m.p. 131-134° C.; IR (solid) 3338 (N-H str), 3262 (N-H str), 1600, 1491, 1468, 1317, 1254, 1166 cm⁻¹; ¹H NMR (300 MHz, DMSO-d₆) δ 7.48 (t, J = 8.0 Hz, 1H, Ar), 7.39 (dt, J = 8.0, 1.5, 1.0 Hz, 1H, Ar), 7.38 - 7.30 (m, 3H, Ar + NH₂), 7.16 (ddd, J = 8.0, 2.5, 1.0 Hz, 1H, Ar), 3.82 (s, 3H, OMe); ¹³C NMR (75.5 MHz, DMSO-d₆) δ 159.2, 145.4, 130.1, 117.6, 117.6, 110.8, 55.5.

N-((3-methoxyphenyl)sulfonyl)-2-(naphthalen-2-yloxy)acetamide (25z). A solution of sulfonamide 28 (185 mg, 0.99 mmol), acid 23 (200 mg, 0.99 mmol), EDCI (228 mg, 1.19 mmol) and DMAP (121 mg, 0.99 mmol) in CH₂Cl₂ (10 mL) was stirred at rt for 26 h. Then, CH₂Cl₂ (25 mL) was added and the resulting solution was washed with 10% HCl (3 × 25 mL), water (25 mL) and brine (25 mL), dried (MgSO₄) and evaporated under reduced pressure to give the crude product. Recrystallisation from MeOH gave 26z (163 mg, 44%) as a white solid, m.p. 151-154° C.; IR (solid) 3313 (N-H str), 1730 (C=O str), 1630, 1601, 1582, 1416, 1258, 1187, 1076 cm⁻¹; ¹H NMR (300 MHz, CDCl₃) δ 8.97 (s, 1H, NH), 7.85 - 7.74 (m, 2H, Ar), 7.65 (d, J = 8.0 Hz 1H, Ar), 7.66 - 7.61 (m, 1H, Ar), 7.57 (dd, J= 2.5, 1.5 Hz, 1H, Ar), 7.47 (ddd, J= 8.0, 7.0, 1.5 Hz, 1H, Ar), 7.41 (ddd, J= 8.0, 7.0, 1.5 Hz, 1H, Ar), 7.40 (dd, J= 8.0, 8.0 Hz, 1H, Ar), 7.18 (dd, J= 6.5, 3.0 Hz, 1H, Ar), 7.16 (ddd, J= 8.5, 2.5, 1.0 Hz, 1H, Ar), 7.00 (d, J= 2.5 Hz, 1H, Ar), 4.60 (s, 2H, OCH₂), 3.81 (s, 3H, OMe); ¹³C NMR (75.5 MHz, CDCl₃) δ 166.2, 159.9, 154.3, 139.2, 134.2, 130.4, 130.2, 129.9, 127.9, 127.2, 127.1, 124.9, 121.2, 120.7, 117.9, 112.8, 107.8, 67.4, 55.8.

Cyclohexylsulfonamide (29). 35% NH₄OH_{(aq)} (3 mL) was added dropwise to a stirred solution of cyclohexylsulfonyl chloride (200 mg, 1.09 mmol) in THF (3 mL) at 0° C. The resulting solution was allowed to warm to rt and stirred at rt for 16 h. Then, water (20 mL) was added and the resulting solution was extracted with EtOAc (3 × 25 mL). The combined organic layers were dried (MgSO₄) and evaporated under reduced pressure to give 29 (91 mg, 51%) as a white solid, m.p. 86-88° C.; IR (solid) 3353 (N-H str), 3255 (N-H str), 2940, 2859, 1313, 1139, 1116 cm⁻¹; ¹H NMR (300 MHz, CDCl₃) δ 4.90 (s, 2H, NH₂), 2.90 (tt, J = 12.0, 3.5 Hz, 1H), 2.21 (ddd, J= 13.0, 3.5, 1.5 Hz, 2H), 1.89 (dt, J= 12.0, 3.0 Hz, 2H), 1.70 (dtt, J = 11.0, 3.0, 1.5 Hz, 1H), 1.48 (qd, J = 12.0, 3.0 Hz, 2H), 1.37 -1.13 (m, 3H); ¹³C NMR (75 MHz, CDCl₃) δ 62.8, 26.6, 25.2.

N-(Cyclohexylsulfonyl)-2-(naphthalen-2-yloxy)acetamide (25aa). A solution of cyclohexylsulfonamide 29 (50 mg, 0.31 mmol), acid 23 (62 mg, 0.31 mmol), EDCI(71 mg, 0.37 mmol) and DMAP (37 mg, 0.31 mmol) in CH₂Cl₂ (5 mL) was stirred at rt for 60 h. Then, CH₂Cl₂ (25 mL) was added and the resulting solution was washed with 10% HCl_{(aq)} (3 × 25 mL), water (25 mL) and brine (25 mL), dried (MgSO₄), and evaporated under reduced pressure to give the crude product. Purification by flash column chromatography on silica with 8:2 petrol:EtOAc as eluent gave 25aa (36 mg, 34%) as a colourless oil, IR (film) 3236 (N-H str), 2934, 2858, 1716 (C=O str), 1630, 1468, 1418, 1390, 1338, 1217, 1180, 1145 cm⁻¹; ¹H NMR (300 MHz, CDCl₃) δ 8.71 (s, 1H, NH), 7.86 - 7.77 (m, 2H, Ar), 7.74 (dd, J= 8.0, 1.0 Hz, 1H, Ar), 7.48 (ddd, J= 8.0, 7.0, 1.5 Hz, 1H, Ar), 7.40 (ddd, J = 8.0, 7.0, 1.5 Hz, 1H, Ar), 7.20 (dd, J = 9.0, 2.5 Hz, 1H, Ar), 7.13 (d, J = 2.5 Hz, 1H, Ar), 4.69 (s, 2H, OCH₂), 3.57 (tt, J = 12.0, 3.5 Hz, 1H, SCH), 2.21 -2.07 (m, 2H), 1.86 (dt, J = 13.0 3.5 Hz, 2H), 1.73 - 1.49 (m, 2H), 1.36 - 1.10 (m, 4H); ¹³C NMR (101 MHz, CDCl₃) δ 167.5 (C), 154.4 (C), 134.2 (C), 130.5 (CH), 129.9 (C), 127.9 (CH), 127.2 (CH), 127.1 (CH), 125.0 (CH), 118.0 (CH), 107.9 (CH), 67.4 (CH₂), 62.1 (CH), 25.8 (CH₂), 25.0 (2 × CH₂).

3-(Furan-3-yl)benzenesulfonamide (31). A solution of 3-bromobenzenesulfonamide (231 mg, 1.0 mmol), furan-3-boronic acid (130 mg, 1.2 mmol), K₂CO₃ (201 mg, 1.5 mmol) and Pd(PPh₃)₂Cl₂ (35 mg, 0.05 mmol) in dioxane (7 mL) and water (0.4 mL) was stirred and heated at reflux for 2 h. Then, the resulting solution was allowed to cool to rt and filtered over a silica plug, washing with EtOAc. The filtrate was evaporated under reduced pressure to give the crude product. Purification by flash column chromatography on silica with 8:2 petrol:EtOAc as eluent gave 3-(furan-3-yl)benzenesulfonamide 31 (241 mg, 86%) as a white solid, m.p. 132-134° C.; IR (solid) 3341 (N-H str), 3259 (N-H str), 1316, 1306, 1157, 1112, 1052, 1013, 904, 872 cm⁻¹; ¹H NMR (300 MHz, CDCl₃) δ 8.03 (dd, J = 2.0, 0.5 Hz, 1H, Ar), 7.84 - 7.79 (m, 2H, Ar), 7.69 (ddd, J = 8.0, 1.5, 1.0 Hz, 1H, Ar), 7.55 (dd, J = 8.0, 0.5 Hz, 1H, Ar), 7.55 - 7.48 (m, 1H, Ar), 6.73 (dd, J= 2.0, 1.0 Hz, 1H, Ar), 4.79 (s, 2H, NH₂); ¹³C NMR (75.5 MHz, CDCl₃) δ 144.4 (CH), 142.7 (C), 139.5 (CH), 134.1 (C), 130.1 (CH), 129.9 (CH), 125.2 (C), 124.8 (CH), 123.8 (CH), 108.7 (CH).

N-3-(Furan-3-yl)phenylsulfonyl-2-(naphthalene-2-yloxy)acetamide (25ab). A solution of sulphonamide 31 (59 mg, 0.28 mmol), acid 23 (57 mg, 0.28 mmol), EDCI (64 mg, 0.34 mmol) and DMAP (34 mg, 0.28 mmol) in CH₂Cl₂ (5 mL) was stirred at rt for 72 h. Then, CH₂Cl₂ (10 mL) was added and the resulting solution was washed with 10% HCl_{(aq)} (3 × 5 mL), water (5 mL) and brine (5 mL), dried (MgSO₄) and evaporated under reduced pressure to give the crude product. Recrystallisation from PhMe/hexane gave 25ab (10 mg, 9%) as a white solid, m.p. 156-158° C.; IR (solid) 3247 (N-H str), 1725 (C=O str), 1630, 1601, 1510, 1416, 1353, 1216, 1160, 839, 750 cm⁻¹; ¹H NMR (400 MHz, CDCl₃) δ 9.02 (br s, 1H, NH), 8.16 (t, J= 2.0 Hz, 1H, Ar), 7.94 (d, J= 8.0 Hz, 1H, Ar), 7.83 - 7.76 (m, 3H, Ar), 7.73 (d, J= 8.0 Hz, 1H, Ar), 7.64 (d, J= 8.0 Hz, 1H, Ar), 7.55 - 7.33 (m, 4H, Ar), 7.18 (dd, J= 9.0, 2.5 Hz, 1H, Ar), 7.00 (d, J= 2.5 Hz, 1H, Ar), 6.71 (d, J= 0.5 Hz, 1H, Ar), 4.60 (s, 2H, CH₂); ¹³ C NMR (101 MHz, CDCl₃) δ 166.3 (C), 154.3 (C), 144.4 (C), 139.6 (CH), 138.8 (C), 134.1 (C), 134.0 (C), 131.5 (CH), 130.4 (CH), 129.9 (C), 129.7 (CH), 127.9 (CH), 127.2 (CH), 127.1 (CH), 126.8 (CH), 125.6 (CH), 124.9 (2 × CH), 117.9 (CH), 108.7 (CH), 107.8 (CH), 67.4 (CH₂); HRMS (ESI) calcd for C₂₂H₁₈NO₅ S 408.0900 (M + H)⁺, found 408.0893.

(1,1′-Biphenyl)-3-sulfonamide (30). A solution of 3-bromobenzenesulfonamide (300 mg, 1.27 mmol), benzeneboronic acid (186 mg, 1.52 mmol), K₂CO₃ (264 mg, 1.91 mmol) and Pd(PPh₃)₂Cl₂ (45 mg, 0.06 mmol) in dioxane (7.5 mL) and water (0.4 mL) was stirred and heated at reflux for 18 h. The resulting solution was allowed to cool to rt and CH₂Cl₂ (10 mL) and water (10 mL) were added. The layers were separated, extracting the aqueous with CH₂Cl₂ (7 × 10 mL). The combined organic layers were dried (MgSO₄) and evaporated under reduced pressure to give the crude product. Purification by flash column chromatography on silica with 7:3 petrol:EtOAc as eluent gave 30 (241 mg, 81%) as a white solid, mp 124-126° C.; IR (solid) 3345 (N-H str), 3246 (N-H str), 1564, 1469, 1408, 1327, 1307, 1287, 1158, 1149, 1092, 905, 894 cm⁻¹; ¹H NMR (400 MHz, CDCl₃) δ 8.16 (t, J= 2.0 Hz, 1H, Ar), 7.90 (d, J= 8.0 Hz, 1H, Ar), 7.79 (d, J = 8.0 Hz, 1H, Ar), 7.63 - 7.35 (m, 6H, Ar), 5.06 (s, 2H, NH₂); ¹³C NMR (101 MHz, CDCl₃) δ 142.7 (C), 142.6 (C), 139.3 (C), 131.5 (CH), 129.8 (CH), 129.2 (CH), 128.4 (CH), 127.3 (2 × CH), 125.1 (CH).Spectroscopic data consistent with that reported in the literature.⁵⁶

N-([1,1′-Biphenyl]-3-ylsulfonyl)-2-(naphthalene-2yloxy)acetamide (25ac). A solution of sulphonamide 30 (204 mg, 0.87 mmol), acid 23 (176 mg, 0.87 mmol), EDCI (203 mg, 1.06 mmol) and DMAP (106 mg, 0.87 mmol) in CH₂Cl₂ (15 mL) was stirred at rt for 72 h. Then, CH₂Cl₂ (30 mL) was added and the resulting solution was washed with 10% HCl_{(aq)} (3 × 15 mL), water (15 mL) and brine (15 mL), dried (MgSO₄) and evaporated under reduced pressure to give the crude product. Purification by flash column chromatography on silica with 7:3 petrol:EtOAc as eluent gave 25ac (49 mg, 13%) as a beige solid, mp 181-182° C.; IR (solid) 3299 (N-H str), 1730 (C=O str), 1628, 1471, 1417, 1354, 1260, 1152, 871, 851, 752 cm⁻¹; ¹H NMR (400 MHz, CDCl₃) δ 9.11 (br s, 1H, NH), 8.32 (t, J= 2.0 Hz, 1H, Ar), 8.03 (dt, J = 8.0, 1.5 Hz, 1H, Ar), 7.85 (dt, J = 8.0, 1.5 Hz, 1H, Ar), 7.81 - 7.72 (m, 3H, Ar), 7.64 (d, J= 8.0 Hz, 1H, Ar), 7.61 - 7.51 (m, 3H, Ar), 7.51 - 7.31 (m, 5H, Ar), 7.18 (dd, J= 9.0, 2.5 Hz, 1H, Ar), 7.01 (d, J= 2.5 Hz, 1H, Ar), 4.59 (s, 2H, CH₂); ¹³C NMR (101 MHz, CDCl₃) δ 166.3 (C), 154.3 (C), 142.5 (C), 139.0 (C), 138.8 (C), 134.1 (C), 133.0 (CH), 130.4 (CH), 129.9 (C), 129.6 (CH), 129.2 (2 × CH), 128.5 (CH), 127.9 (CH), 127.4 (2 × CH), 127.2 (CH), 127.1 (CH), 127.1 (CH), 127.1 (CH), 124.9 (CH), 117.9 (CH), 107.8 (CH), 67.3 (CH₂); HRMS (ESI) calcd for C₂₄H₂₀NO₄ S 418.1108 (M + H)⁺, found 418.1109.

Methyl 3-(N-(2-(naphthalene-2-yloxy)acetyl)sulfamoyl)benzoate (25ad). A solution of methyl 3-sulfamoylbenzoate (158 mg, 0.73 mmol), acid 23 (148 mg, 0.73 mmol), EDCI (169 mg, 0.88 mmol) and DMAP (90 mg, 0.73 mmol) in CH₂Cl₂ (13 mL) was stirred at rt for 72 h. CH₂Cl₂ (25 mL) was added and the resulting solution was washed with 10% HCl_{(aq)} (3 × 15 mL), water (15 mL) and brine (15 mL), dried (MgSO₄) and evaporated under reduced pressure to give the crude product. Recrystallisation from acetone/hexane gave 25ad (103 mg, 35%) as a white solid, mp 104-106° C.; IR (solid) 3278 (N-H str), 3071, 1718 (br, 2 × C=O str), 1629, 1603, 1511, 1422, 1365, 1304, 1275, 1265, 1167, 1131, 1068, 866, 848, 753 cm⁻¹; ¹H NMR (400 MHz, CDCl₃) δ 8.68 (t, J = 2.0 Hz, 1H, Ar), 8.27 (t, J = 8.0 Hz, 2H, Ar), 7.77 (d, J = 8.5 Hz, 2H, Ar), 7.67 - 7.53 (m, 2H, Ar), 7.50 - 7.28 (m, 2H, Ar), 7.17 (dd, J = 9.0, 2.5 Hz, 1H, Ar), 6.96 (d, J= 2.5 Hz, 1H, Ar), 4.58 (s, 2H, CH₂), 3.91 (s, 3H, CH₃); ¹³C NMR (101 MHz, CDCl₃) δ 166.5 (C), 165.2 (C), 154.3 (C), 138.7 (C), 135.2 (CH), 134.1 (C), 132.8 (CH), 131.4 (C), 130.4 (CH), 129.8 (C), 129.6 (CH), 129.4 (CH), 127.8 (CH), 127.1 (CH), 127.1 (CH), 124.9 (CH), 117.9 (CH), 107.7 (CH), 67.3 (CH₂), 52.8 (CH₃); HRMS (ESI) calcd for C₂₀H₁₈NO₆S 400.0849 (M + H)⁺, found 400.0859.

Recombinant Protein Purification. Recombinant EPAC1-CNBD (169-318), EPAC2-CNBD (304-453), EPAC1-ΔDEP (149-881), EPAC2-ΔDEP (280-993) and RalGDS-RBD (aa 788-884) cloned into vectors ofpGEX series were expressed as glutathione-S transferase (GST) fusion proteins in chemically competent Escherichia coli (E. coli) strain BL21 Star™ (DE3) One Shot® (Invitrogen). Expression and purification procedures were based on previously described methods.⁵¹

8-NBD-cAMP Competition Binding Assay. The previously described fluorescence-based 8-NBD-cAMP competition binding assay was used to screen compound 3 analogues for binding to the EPAC1-CNBD.^{51,}⁵⁷ Experiments were carried out in black 96-well plates in Assay Buffer (50 mM Tris-HCl, pH = 7.5, 50 mM NaCl, 1 mM EDTA, 1 mM DTT). Studied compounds, EPAC1-CNBD and 8-NBD-cAMP were combined at 10 µM, 0.8 µM and 62.5 nM concentrations, respectively. Eleven-point dose-response experiments were performed on compound 3 and selected analogues to compare their binding to EPAC1-CNBD and EPAC1-ΔDEP. Experiments were carried out in black 96-well plates in Assay Buffer. Studied compounds, proteins and 8-NBD-cAMP were combined at 1-100 µM, 0.8 µM and 62.5 nM concentrations, respectively. Plates were then incubated for 4 h at room temperature, protected from light. Fluorescence intensity was then measured using a FLUOstar Omega microplate reader (BMG LABTECH) at excitation/emission wavelengths of 485/520 nm. Relative fluorescence intensity (RFI) = (Fluorescence intensity of studied compounds, EPAC1-CNBD/EPAC1-ΔDEP and 8-NBD-cAMP combined at 10 µM, 0.8 µM and 62.5 nM concentrations) ÷ (Fluorescence intensity of EPAC1-CNBD/EPAC1-ΔDEP and 8-NBD-cAMP combined at 0.8 µM and 62.5 nM concentrations) × 100%

Active Rap1 Pull-down. U2OS cells (from Professor Holger Rehmann, University of Utrecht) stably transfected with EPAC1 or EPAC2 were cultured in 6-well plates in DMEM, high glucose, supplemented with 10% (v/v) FBS, 1% (v/v) GlutaMAX, 1% (v/v) Penicillin-Streptomycin and 2 mg/l puromycin (to ensure selection of stable transfectants). 80% confluent cells were starved in culture medium with reduced FBS concentration (0.5 %) for 16 h and then stimulated for 10 min with either vehicle, 100 µM of studied compounds, or 50 µM of 2 in case of U2OS-EPAC1 or 100 µM of compound 4 for U2OS-EPAC2. Cells were then rinsed with ice-cold PBS and lysed in 0.5 ml cell lysis buffer (Cell Signaling Technologies) supplemented with 10 mM MgCl₂ and 1 mM PMSF, followed by clearing the lysates by centrifugation. Cell lysates were incubated for 1 h (4° C., gentle agitation) with 40 µg GST-RalGDS-RBD immobilized on Glutathione Sepharose 4B (GE Healthcare) to selectively capture GTP-bound Rap1. Later on, the glutathione resin was separated from supernatant by centrifugation, washed three times with cell lysis buffer, then resuspended in 2x SDS sample loading buffer and denatured for 5 min at 95° C.

SDS-PAGE and Western Blotting. Samples were prepared by mixing equal volumes of cell lysate and 2x SDS sample loading buffer and denaturing for 5 min at 95° C., unless indicated otherwise. Protein samples were separated by SDS-PAGE on 10% (v/v) polyacrylamide gels, for EPAC1 and VASP, or on 12.5% (v/v), for Rap1, and then transferred to nitrocellulose membranes. Membranes were then blocked for 1 h at room temperature in 5% (w/v) non-fat dry milk or 5% (w/v) BSA in Tris-buffered saline containing 0.1% (v/v) Tween 20, followed by an overnight incubation with primary antibody diluted in blocking buffer at 4° C. Subsequently, the membranes were incubated with appropriate horseradish peroxidase-conjugated secondary antibodies for 1 h at room temperature. For signal detection the SuperSignal West Pico PLUS Chemiluminescent Substrate (Thermo Scientific) was used. Images were acquired using the Fusion FX7 camera platform (Vilber). Densitometry was performed with ImageJ.

REFERENCES
1. Robichaux, W. G., III; Cheng, X. Intracellular cAMP sensor EPAC: physiology, pathophysiology, and therapeutics development. Physiol. Rev. 2018, 98, 919-1053.
2. Cohen, P. Protein kinases--the major drug targets of the twenty-first century? Nat. Rev. Drug Discov. 2002, 1, 309-315.
3. Taylor, S. S.; Zhang, P.; Steichen, J. M.; Keshwani, M. M.; Kornev, A. P. PKA: lessons learned after twenty years. Biochim. Biophys. Acta. 2013, 1834, 1271-1278.
4. Torres-Quesada, O.; Mayrhofer, J. E.; Stefan, E. The many faces of compartmentalized PKA signalosomes. Cell. Signal. 2017, 37, 1-11.
5. Biel, M. Cyclic nucleotide-regulated cation channels. J. Biol. Chem. 2009, 284, 9017-9021.
6. Bos, J. L. Epac: a new cAMP target and new avenues in cAMP research. Nat. Rev. Mol. Cell Biol. 2003, 4, 733-738.
7. Cheng, X.; Ji, Z.; Tsalkova, T.; Mei, F. Epac and PKA: a tale of two intracellular cAMP receptors. Acta. Biochim. Biophys. Sin. (Shanghai) 2008, 40, 651-662.
8. Chen, H.; Wild, C.; Zhou, X.; Ye, N.; Cheng, X.; Zhou, J. Recent Advances in the Discovery of Small Molecules Targeting Exchange Proteins Directly Activated by cAMP (EPAC). J. Med. Chem. 2014, 57, 3651-3665.
9. Parnell, E.; Palmer, T. M.; Yarwood, S. J. The future of EPAC-targeted therapies: agonism versus antagonism. Trends Pharmacol. Sci. 2015, 36, 203-214.
10. Wang, P.; Liu, Z.; Chen, H.; Ye, N.; Cheng, X.; Zhou, J. Exchange proteins directly activated by cAMP (EPACs): Emerging therapeutic targets. Bioorg. Med. Chem. Lett. 2017, 27, 1633-1639.
11. de Rooij, J.; Zwartkruis, F. J.; Verheijen, M. H.; Cool, R. H.; Nijman, S. M.; Wittinghofer, A.; Bos, J. L. Epac is a Rap1 guanine-nucleotide-exchange factor directly activated by cyclic AMP. Nature 1998, 396, 474-477.
12. Kawasaki, H.; Springett, G. M.; Mochizuki, N.; Toki, S.; Nakaya, M.; Matsuda, M.; Housman, D. E.; Graybiel, A. M. A family of cAMP-binding proteins that directly activate Rap1. Science 1998, 282, 2275-2279.
13. Banerjee, U.; Cheng, X. Exchange protein directly activated by cAMP encoded by the mammalian rapgef3 gene: Structure, function and therapeutics. Gene 2015, 570, 157-167.
14. Rehmann, H.; Das, J.; Knipscheer, P.; Wittinghofer, A.; Bos, J. L. Structure of the cyclic-AMP-responsive exchange factor Epac2 in its auto-inhibited state. Nature 2006, 439, 625-628.
15. Rehmann, H.; Arias-Palomo, E.; Hadders, M. A.; Schwede, F.; Llorca, O.; Bos, J. L. Structure of Epac2 in complex with a cyclic AMP analogue and RAP1B. Nature 2008, 455, 124-127.
16. Laudette, M.; Zuo, H.; Lezoualc’h, F.; Schmidt, M. Epac function and cAMP scaffolds in the heart and lung. J. Cardiovasc. Dev. Dis. 2018, 5, 50/51-50/16.
17. Ye, N.; Zhu, Y.; Liu, Z.; Mei, F. C.; Chen, H.; Wang, P.; Cheng, X.; Zhou, J. Identification of novel 2-(benzo[d]isoxazol-3-yl)-2-oxo-N-phenylacetohydrazonoyl cyanide analoguesas potent EPAC antagonists. Eur. J. Med. Chem. 2017, 134, 62-71.
18. Liu, Z.; Zhu, Y.; Chen, H.; Wang, P.; Mei, F. C.; Ye, N.; Cheng, X.; Zhou, J. Structure-activity relationships of 2-substituted phenyl-N-phenyl-2-oxoacetohydrazonoyl cyanides as novel antagonists of exchange proteins directly activated by cAMP (EPACs). Bioorg. Med. Chem. Lett. 2017, 27, 5163-5166.
19. Wild, C. T.; Zhu, Y.; Na, Y.; Mei, F.; Ynalvez, M. A.; Chen, H.; Cheng, X.; Zhou, J. Functionalized N,N-Diphenylamines as Potent and Selective EPAC2 Inhibitors. ACS Med. Chem. Lett. 2016, 7, 460-464.
20. Zhu, Y.; Chen, H.; Boulton, S.; Mei, F.; Ye, N.; Melacini, G.; Zhou, J.; Cheng, X. Biochemical and pharmacological characterizations of ESI-09 based EPAC inhibitors: defining the ESI-09 “therapeutic window”. Sci. Rep. 2015, 5, 9344.
21. Ye, N.; Zhu, Y.; Chen, H.; Liu, Z.; Mei, F. C.; Wild, C.; Chen, H.; Cheng, X.; Zhou, J. Structure-Activity Relationship Studies of Substituted 2-(Isoxazol-3-yl)-2-oxo-N′-phenyl-acetohydrazonoyl Cyanide Analogues: Identification of Potent Exchange Proteins Directly Activated by cAMP (EPAC) Antagonists. J. Med. Chem. 2015, 58, 6033-6047.
22. Chen, H.; Tsalkova, T.; Chepurny, O. G.; Mei, F. C.; Holz, G. G.; Cheng, X.; Zhou, J. Identification and characterization of small molecules as potent and specific EPAC2 antagonists. J. Med. Chem. 2013, 56, 952-962.
23. Chen, H.; Ding, C.; Wild, C.; Liu, H.; Wang, T.; White, M. A.; Cheng, X.; Zhou, J. Efficient Synthesis of ESI-09, A Novel Non-cyclic Nucleotide EPAC Antagonist. Tetrahedron Lett. 2013, 54, 1546-1549.
24. Chen, H.; Tsalkova, T.; Mei, F. C.; Hu, Y.; Cheng, X.; Zhou, J. 5-Cyano-6-oxo-1,6-dihydro-pyrimidines as potent antagonists targeting exchange proteins directly activated by cAMP. Bioorg. Med. Chem. Lett. 2012, 22, 4038-4043.
25. Kumar, N.; Prasad, P.; Jash, E.; Saini, M.; Husain, A.; Goldman, A.; Sehrawat, S. Insights into exchange factor directly activated by cAMP (EPAC) as potential target for cancer treatment. Mol. Cell. Biochem. 2018, 447, 77-92.
26. Gong, B.; Shelite, T.; Mei, F. C.; Ha, T.; Hu, Y.; Xu, G.; Chang, Q.; Wakamiya, M.; Ksiazek, T. G.; Boor, P. J.; Bouyer, D. H.; Popov, V. L.; Chen, J.; Walker, D. H.; Cheng, X. Exchange protein directly activated by cAMP plays a critical role in bacterial invasion during fatal rickettsioses. Proc. Natl. Acad. Sci. U. S. A. 2013, 110, 19615-19620.
27. Choi, E.-J.; Ren, Y.; Chen, Y.; Liu, S.; Wu, W.; Ren, J.; Wang, P.; Garofalo, R. P.; Zhou, J.; Bao, X. Exchange proteins directly activated by cAMP and their roles in respiratory syncytial virus infection. J. Virol. 2018, 92, e01200-01218.
28. Yan, J.; Mei, F. C.; Cheng, H.; Lao, D. H.; Hu, Y.; Wei, J.; Patrikeev, I.; Hao, D.; Stutz, S. J.; Dineley, K. T.; Motamedi, M.; Hommel, J. D.; Cunningham, K. A.; Chen, J.; Cheng, X. Enhanced leptin sensitivity, reduced adiposity, and improved glucose homeostasis in mice lacking exchange protein directly activated by cyclic AMP isoform 1. Mol. Cell. Biol. 2013, 33, 918-926.
29. Wang, H.; Heijnen, C. J.; van Velthoven, C. T.; Willemen, H. L.; Ishikawa, Y.; Zhang, X.; Sood, A. K.; Vroon, A.; Eijkelkamp, N.; Kavelaars, A. Balancing GRK2 and EPAC1 levels prevents and relieves chronic pain. J. Clin. Invest. 2013, 123, 5023-5034.
30. Zhou, L.; Ma, S. L.; Yeung, P. K.; Wong, Y. H.; Tsim, K. W.; So, K. F.; Lam, L. C.; Chung, S. K. Anxiety and depression with neurogenesis defects in exchange protein directly activated by cAMP 2-deficient mice are ameliorated by a selective serotonin reuptake inhibitor, Prozac. Transl. Psychiatry. 2016, 6, e881.
31. Liu, L.; Jiang, Y.; Steinle, J. J. Epac1 and Glycyrrhizin Both Inhibit HMGB1 Levels to Reduce Diabetes-Induced Neuronal and Vascular Damage in the Mouse Retina. J. Clin. Med. 2019, 8.
32. Christensen, A. E.; Selheim, F.; de Rooij, J.; Dremier, S.; Schwede, F.; Dao, K. K.; Martinez, A.; Maenhaut, C.; Bos, J. L.; Genieser, H. G.; Doskeland, S. O. cAMP analog mapping of Epac1 and cAMP kinase. Discriminating analogs demonstrate that Epac and cAMP kinase act synergistically to promote PC-12 cell neurite extension. J. Biol. Chem. 2003, 278, 35394-35402.
33. Kiermayer, S.; Biondi, R. M.; Imig, J.; Plotz, G.; Haupenthal, J.; Zeuzem, S.; Piiper, A. Epac activation converts cAMP from a proliferative into a differentiation signal in PC12 cells. Mol. Biol. Cell 2005, 16, 5639-5648.
34. Emery, A. C.; Eiden, M. V.; Eiden, L. E. Separate cyclic AMP sensors for neuritogenesis, growth arrest, and survival of neuroendocrine cells. J. Biol. Chem. 2014, 289, 10126-10139.
35. Yang, Y.; Shu, X.; Liu, D.; Shang, Y.; Wu, Y.; Pei, L.; Xu, X.; Tian, Q.; Zhang, J.; Qian, K.; Wang, Y. X.; Petralia, R. S.; Tu, W.; Zhu, L. Q.; Wang, J. Z.; Lu, Y. EPAC null mutation impairs learning and social interactions via aberrant regulation of miR-124 and Zif268 translation. Neuron 2012, 73, 774-788.
36. Yarwood, S. J.; Borland, G.; Sands, W. A.; Palmer, T. M. Identification of CCAAT/enhancer-binding proteins as exchange protein activated by cAMP-activated transcription factors that mediate the induction of the SOCS-3 gene. J. Biol. Chem. 2008, 283, 6843-6853.
37. Parnell, E.; Smith, B. O.; Palmer, T. M.; Terrin, A.; Zaccolo, M.; Yarwood, S. J. Regulation of the inflammatory response of vascular endothelial cells by EPAC1. Br. J. Pharmacol. 2012, 166, 434-446.
38. Borland, G.; Smith, B. O.; Yarwood, S. J. EPAC proteins transduce diverse cellular actions of cAMP. Br. J. Pharmacol. 2009, 158, 70-86.
39. Jiang, Y.; Liu, L.; Curtiss, E.; Steinle, J. J. Epac1 Blocks NLRP3 Inflammasome to Reduce IL-1beta in Retinal Endothelial Cells and Mouse Retinal Vasculature. Mediators Inflamm. 2017, 2017, 2860956.
40. Liu, L.; Jiang, Y.; Chahine, A.; Curtiss, E.; Steinle, J. J. Epac1 agonist decreased inflammatory proteins in retinal endothelial cells, and loss of Epac1 increased inflammatory proteins in the retinal vasculature of mice. Mol. Vis. 2017, 23, 1-7.
41. Fujita, T.; Umemura, M.; Yokoyama, U.; Okumura, S.; Ishikawa, Y. The role of Epac in the heart. Cell Mol. Life Sci. 2017, 74, 591-606.
42. Laubner, K.; Kieffer, T. J.; Lam, N. T.; Niu, X.; Jakob, F.; Seufert, J. Inhibition of preproinsulin gene expression by leptin induction of suppressor of cytokine signaling 3 in pancreatic beta-cells. Diabetes 2005, 54, 3410-3417.
43. Wan, X.; Torregrossa, M. M.; Sanchez, H.; Nairn, A. C.; Taylor, J. R. Activation of exchange protein activated by cAMP in the rat basolateral amygdala impairs reconsolidation of a memory associated with self-administered cocaine. PLoS One 2014, 9, e107359.
44. Eijkelkamp, N.; Wang, H.; Garza-Carbajal, A.; Willemen, H. L.; Zwartkruis, F. J.; Wood, J. N.; Dantzer, R.; Kelley, K. W.; Heijnen, C. J.; Kavelaars, A. Low nociceptor GRK2 prolongs prostaglandin E2 hyperalgesia via biased cAMP signaling to Epac/Rap1, protein kinase Cepsilon, and MEK/ERK. J. Neurosci. 2010, 30, 12806-12815.
45. Lezoualc’h, F.; Fazal, L.; Laudette, M.; Conte, C. Cyclic AMP Sensor EPAC Proteins and Their Role in Cardiovascular Function and Disease. Circ. Res. 2016, 118, 881-897.
46. Barker, G.; Parnell, E.; van Basten, B.; Buist, H.; Adams, D. R.; Yarwood, S. J. The potential of a novel class of EPAC-selective agonists to combat cardiovascular inflammation. J. Cardiovasc. Dev. Dis. 2017, 4, 22/21-22/16.
47. Enserink, J. M.; Christensen, A. E.; de Rooij, J.; van Triest, M.; Schwede, F.; Genieser, H. G.; Doskeland, S. O.; Blank, J. L.; Bos, J. L. A novel Epac-specific cAMP analogue demonstrates independent regulation of Rap1 and ERK. Nat. Cell Biol. 2002, 4, 901-906.
48. Schwede, F.; Bertinetti, D.; Langerijs, C. N.; Hadders, M. A.; Wienk, H.; Ellenbroek, J. H.; de Koning, E. J.; Bos, J. L.; Herberg, F. W.; Genieser, H. G.; Janssen, R. A.; Rehmann, H. Structure-guided design of selective Epac1 and Epac2 agonists. PLoS Biol. 2015, 13, e1002038.
49. Tsalkova, T.; Mei, F. C.; Li, S.; Chepurny, O. G.; Leech, C. A.; Liu, T.; Holz, G. G.; Woods, V. L., Jr.; Cheng, X. Isoform-specific antagonists of exchange proteins directly activated by cAMP. Proc. Natl. Acad. Sci. U. S. A. 2012, 109, 18613-18618.
50. Tsalkova, T.; Mei, F. C.; Cheng, X. A fluorescence-based high-throughput assay for the discovery of exchange protein directly activated by cyclic AMP (EPAC) antagonists. PLoS One 2012, 7, e30441.
51. Parnell, E.; McElroy, S. P.; Wiejak, J.; Baillie, G. L.; Porter, A.; Adams, D. R.; Rehmann, H.; Smith, B. O.; Yarwood, S. J. Identification of a Novel, Small Molecule Partial Agonist for the Cyclic AMP Sensor, EPAC1. Sci. Rep. 2017, 7, 294.
52. Besnard, J.; Ruda, G. F.; Setola, V.; Abecassis, K.; Rodriguiz, R. M.; Huang, X. P.; Norval, S.; Sassano, M. F.; Shin, A. I.; Webster, L. A.; Simeons, F. R.; Stojanovski, L.; Prat, A.; Seidah, N. G.; Constam, D. B.; Bickerton, G. R.; Read, K. D.; Wetsel, W. C.; Gilbert, I. H.; Roth, B. L.; Hopkins, A. L. Automated design of ligands to polypharmacological profiles. Nature 2012, 492, 215-220.
53. Feng, J. B.; Wu, X. F. A general iodine-mediated synthesis of primary sulfonamides from thiols and aqueous ammonia. Org. Biomol. Chem. 2016, 14, 6951-6954.
54. Arakawa, Y.; Nakajima, S.; Kang, S.; Shigeta, M.; Konishi, G.-i.; Watanabe, J. Synthesis and evaluation of dinaphthylacetylene nematic liquid crystals for high-birefringence materials. Liquid Crystals 2012, 39, 1063-1069.
55. Han, S. H.; Pandey, A.; Lee, H.; Kim, S.; Kang, D.; Jung, Y.; Kim, N.-H.; Hong, S.; Kim, I. One-pot Synthesis of 2-Naphthols from Nitrones and MBH Adducts via Decarboxylative N-O Bond Cleavage. Org. Chem. Front. 2018, 5, 3210- 3218.
56. Charlton, M. H.; Aleksis, R.; Saint-Leger, A.; Gupta, A.; Loza, E.; Ribas de Pouplana, L.; Kaula, I.; Gustina, D.; Madre, M.; Lola, D.; Jaudzems, K.; Edmund, G.; Randall, C. P.; Kime, L.; O’Neill, A. J.; Goessens, W.; Jirgensons, A.; Finn, P. W. N-Leucinyl Benzenesulfonamides as Structurally Simplified Leucyl-tRNA Synthetase Inhibitors. ACS Med. Chem. Lett. 2018, 9, 84-88.
57. Zakrzewicz, D.; Didiasova, M.; Giaimo, B. D.; Borggrefe, T.; Preissner, K. T.; Kruger, M.; Mieth, M.; Hocke, A. C.; Zakrzewicz, A.; Schaefer, L.; Wygrecka, M. Protein arginine methyltransferase 5 mediates enolase-1 cell surface trafficking in human lung adenocarcinoma cells. Biochim. Biophys. Acta. 2018, 1864, 1816-1827.

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.

1. A compound according to Formula I or a pharmaceutically acceptable salt thereof, wherein: 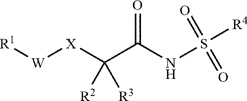
wherein:
R¹ is independently chosen from H, alkyl, alkoxy, halogen, cyan, amino, hydroxyl, NO₂, CF₃ and -OCF₃;
W is independently chosen from forming a 5-12 membered aryl, heteroaryl and heterocycle having 1-3 heteroatoms;
X is independently chosen from O, S, NH and CH₂;
or W and X are optionally joined to form a 5-12 membered heteroaryl or heterocycle having 1-3 heteroatoms and optionally substituted with one or more substituents selected from H, alkyl, alkoxy, halogen, cyan, amino, NO₂, hydroxyl, CF₃ and —OCF₃;
R² and R³ is independently chosen from H, alkyl and F;
R⁴ is 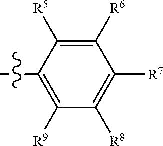 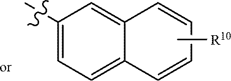
wherein R⁵, R⁶, R⁷, R⁸, R⁹ and R¹⁰ is independently chosen from H, alkyl, cycloalkyl, alkenyl, aryl, heteroaryl, benzyl, alkoxy, halogen, cyan, nitro, amino, hydroxyl, CF₃ and —OCF₃, wherein R⁵, R⁶, R⁷, R⁸, R⁹ and R¹⁰ is optionally substituted with one or more chosen substituents chosen from hydroxyl, cyan, amino, halogen, heteroaryl and heterocycle, wherein said heteroaryl and said heterocycle is optionally substituted with one or more substituents selected from H, alkyl, alkoxy, halogen, cyan, amino, NO₂, hydroxyl, CF₃ and —OCF₃.

2. A compound according to Formula II or a pharmaceutically acceptable salt thereof, wherein: 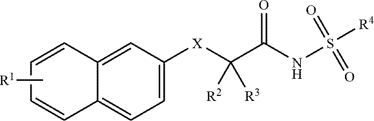
wherein:
R¹ is independently chosen from H, alkyl, alkoxy, halogen, cyan, amino, hydroxyl, nitro, CF₃ and -OCF₃;
X is independently chosen from O, S, NH and CH₂;
R² and R³ is independently chosen from H, alkyl and F;
R⁴ is 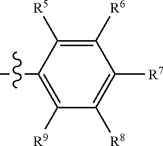 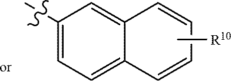
wherein R⁵, R⁶, R⁷, R⁸, R⁹ and R¹⁰ is independently chosen from H, alkyl, cycloalkyl, alkenyl, aryl, heteroaryl, benzyl, alkoxy, halogen, cyan, nitro, amino, hydroxyl, CF₃ and —OCF₃, wherein R⁵, R⁶, R⁷, R⁸, R⁹ and R¹⁰ is optionally substituted with one or more chosen substituents chosen from hydroxyl, cyan, amino, halogen, heteroaryl or heterocycle, wherein heteroaryl or heterocycle is optionally substituted with one or more substituents selected from H, alkyl, alkoxy, halogen, cyan, amino, NO₂, hydroxyl, CF₃ or —OCF_{3.}

3. The compound according to claim 2, wherein R² and R³ are H, and 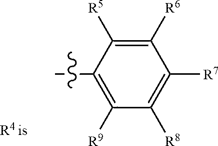 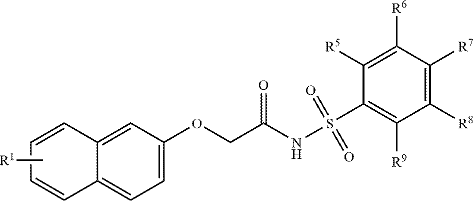
or
wherein R² and R³ are H, and R⁴ is

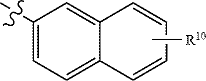

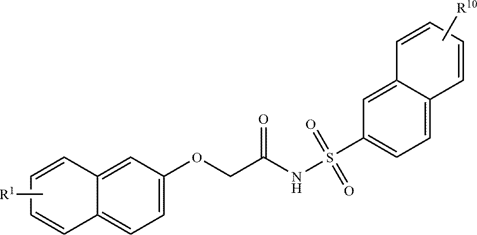

4. The compound according to claim 2, wherein the compound is: 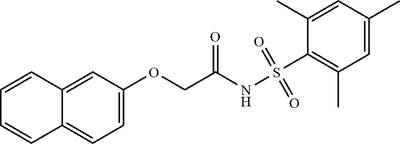 [SMILES: Cc1cc(C)c(S(=O)(=O)NC(=O)COc2ccc3ccccc3c2)c(C)c1] 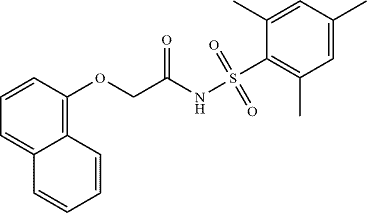 [SMILES: Cc1cc(C)c(S(=O)(=O)NC(=O)COc2cccc3ccccc23)c(C)c1] 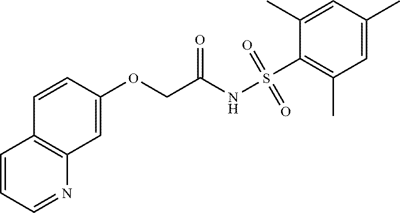 [SMILES: Cc1cc(C)c(S(=O)(=O)NC(=O)COc2ccc3cccnc3c2)c(C)c1] 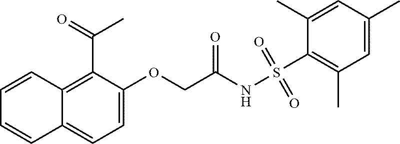 [SMILES: CC(=O)c1c(OCC(=O)NS(=O)(=O)c2c(C)cc(C)cc2C)ccc2ccccc12] 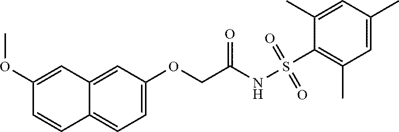 [SMILES: COc1ccc2ccc(OCC(=O)NS(=O)(=O)c3c(C)cc(C)cc3C)cc2c1] 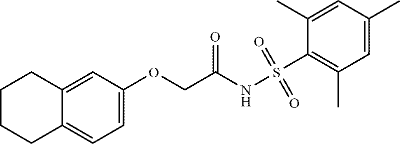 [SMILES: Cc1cc(C)c(S(=O)(=O)NC(=O)COc2ccc3c(c2)CCCC3)c(C)c1] 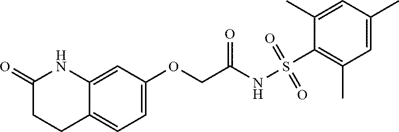 [SMILES: Cc1cc(C)c(S(=O)(=O)NC(=O)COc2ccc3c(c2)NC(=O)CC3)c(C)c1] 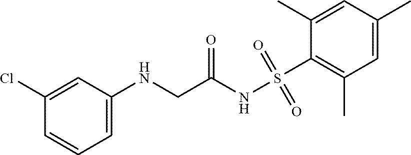 [SMILES: Cc1cc(C)c(S(=O)(=O)NC(=O)CNc2cccc(Cl)c2)c(C)c1] 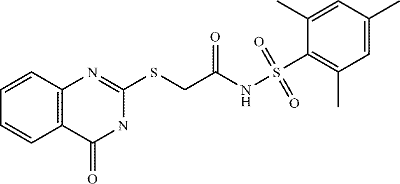 [SMILES: Cc1cc(C)c(S(=O)(=O)NC(=O)CSc2nc3ccccc3c(=O)[nH]2)c(C)c1] 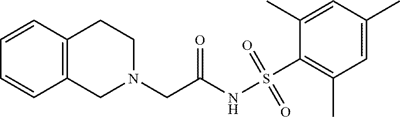 [SMILES: Cc1cc(C)c(S(=O)(=O)NC(=O)CN2CCc3ccccc3C2)c(C)c1] 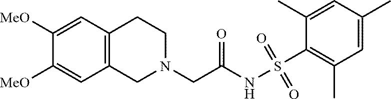 [SMILES: COc1cc2c(cc1OC)CN(CC(=O)NS(=O)(=O)c1c(C)cc(C)cc1C)CC2] 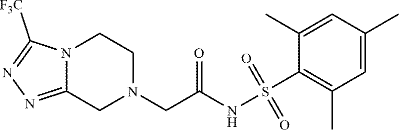 [SMILES: Cc1cc(C)c(S(=O)(=O)NC(=O)CN2CCn3c(nnc3C(F)(F)F)C2)c(C)c1] 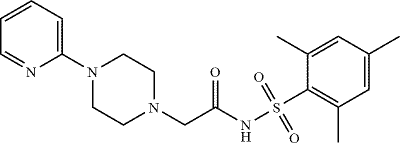 [SMILES: Cc1cc(C)c(S(=O)(=O)NC(=O)CN2CCN(c3ccccn3)CC2)c(C)c1] 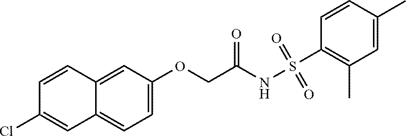 [SMILES: Cc1ccc(S(=O)(=O)NC(=O)COc2ccc3cc(Cl)ccc3c2)c(C)c1] 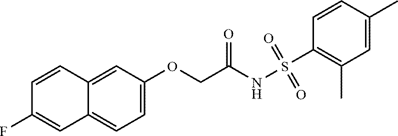 [SMILES: Cc1ccc(S(=O)(=O)NC(=O)COc2ccc3cc(F)ccc3c2)c(C)c1] 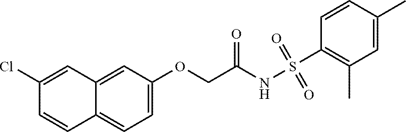 [SMILES: Cc1ccc(S(=O)(=O)NC(=O)COc2ccc3ccc(Cl)cc3c2)c(C)c1] 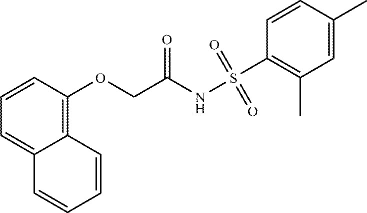 [SMILES: Cc1ccc(S(=O)(=O)NC(=O)COc2cccc3ccccc23)c(C)c1] 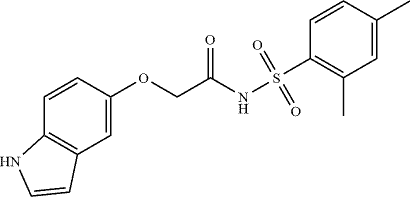 [SMILES: Cc1ccc(S(=O)(=O)NC(=O)COc2ccc3[nH]ccc3c2)c(C)c1] 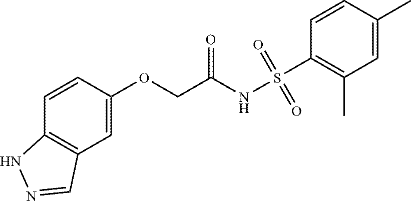 [SMILES: Cc1ccc(S(=O)(=O)NC(=O)COc2ccc3[nH]ncc3c2)c(C)c1] 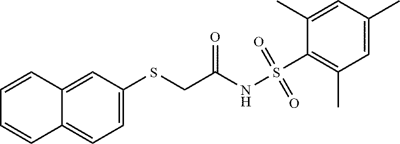 [SMILES: Cc1cc(C)c(S(=O)(=O)NC(=O)CSc2ccc3ccccc3c2)c(C)c1] 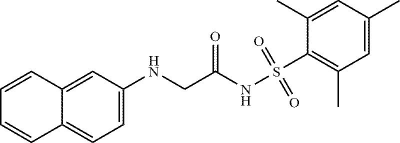 [SMILES: Cc1cc(C)c(S(=O)(=O)NC(=O)CNc2ccc3ccccc3c2)c(C)c1] 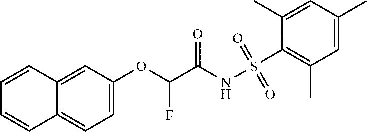 [SMILES: Cc1cc(C)c(S(=O)(=O)NC(=O)C(F)Oc2ccc3ccccc3c2)c(C)c1] 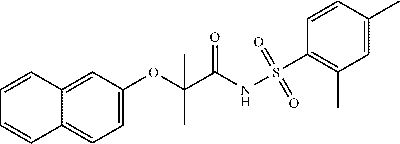 [SMILES: Cc1ccc(S(=O)(=O)NC(=O)C(C)(C)Oc2ccc3ccccc3c2)c(C)c1] 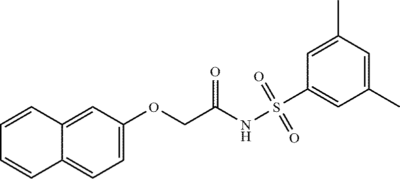 [SMILES: Cc1cc(C)cc(S(=O)(=O)NC(=O)COc2ccc3ccccc3c2)c1] 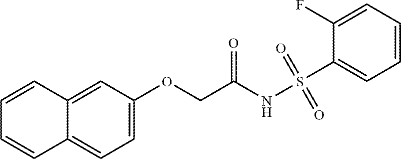 [SMILES: O=C(COc1ccc2ccccc2c1)NS(=O)(=O)c1ccccc1F] 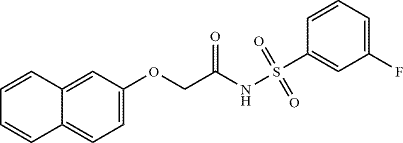 [SMILES: O=C(COc1ccc2ccccc2c1)NS(=O)(=O)c1cccc(F)c1] 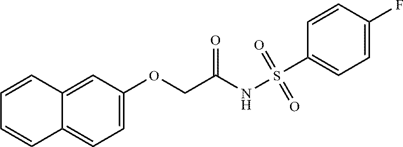 [SMILES: O=C(COc1ccc2ccccc2c1)NS(=O)(=O)c1ccc(F)cc1] 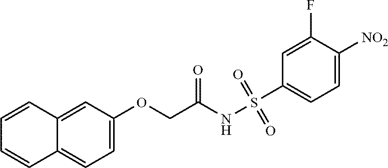 [SMILES: O=C(COc1ccc2ccccc2c1)NS(=O)(=O)c1ccc([N+](=O)[O-])c(F)c1] 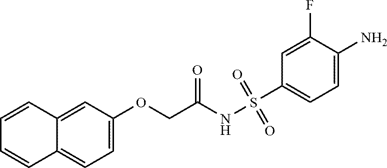 [SMILES: Nc1ccc(S(=O)(=O)NC(=O)COc2ccc3ccccc3c2)cc1F] 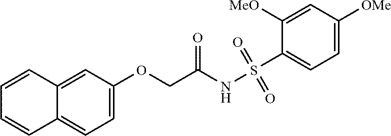 [SMILES: COc1ccc(S(=O)(=O)NC(=O)COc2ccc3ccccc3c2)c(OC)c1] 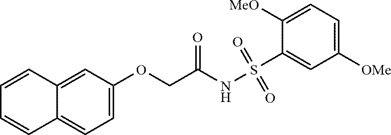 [SMILES: COc1ccc(OC)c(S(=O)(=O)NC(=O)COc2ccc3ccccc3c2)c1] 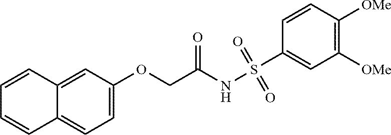 [SMILES: COc1ccc(S(=O)(=O)NC(=O)COc2ccc3ccccc3c2)cc1OC] 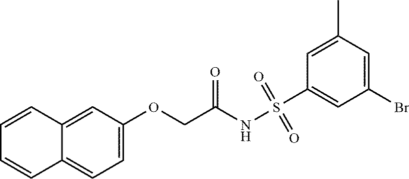 [SMILES: Cc1cc(Br)cc(S(=O)(=O)NC(=O)COc2ccc3ccccc3c2)c1] 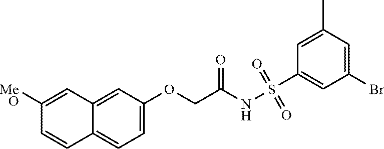 [SMILES: COc1ccc2ccc(OCC(=O)NS(=O)(=O)c3cc(C)cc(Br)c3)cc2c1] 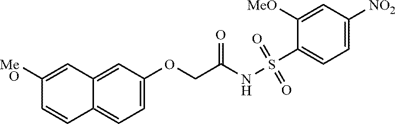 [SMILES: COc1ccc2ccc(OCC(=O)NS(=O)(=O)c3ccc([N+](=O)[O-])cc3OC)cc2c1] 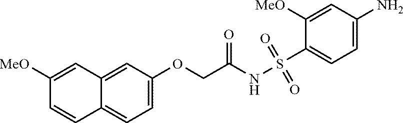 [SMILES: COc1ccc2ccc(OCC(=O)NS(=O)(=O)c3ccc(N)cc3OC)cc2c1] 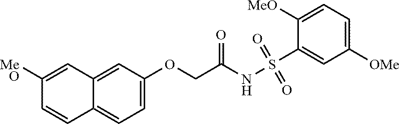 [SMILES: COc1ccc(OC)c(S(=O)(=O)NC(=O)COc2ccc3ccc(OC)cc3c2)c1] 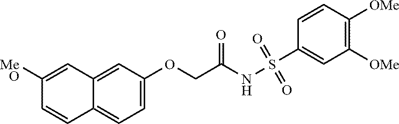 [SMILES: COc1ccc2ccc(OCC(=O)NS(=O)(=O)c3ccc(OC)c(OC)c3)cc2c1] 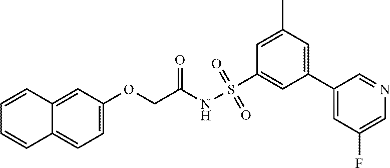 [SMILES: Cc1cc(-c2cncc(F)c2)cc(S(=O)(=O)NC(=O)COc2ccc3ccccc3c2)c1] 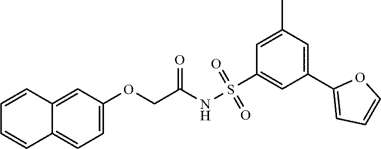 [SMILES: Cc1cc(-c2ccco2)cc(S(=O)(=O)NC(=O)COc2ccc3ccccc3c2)c1] 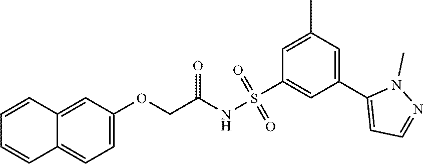 [SMILES: Cc1cc(-c2ccnn2C)cc(S(=O)(=O)NC(=O)COc2ccc3ccccc3c2)c1] 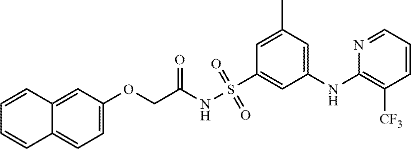 [SMILES: Cc1cc(Nc2ncccc2C(F)(F)F)cc(S(=O)(=O)NC(=O)COc2ccc3ccccc3c2)c1] 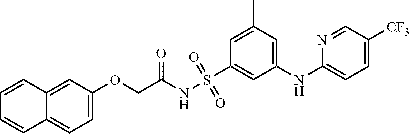 [SMILES: Cc1cc(Nc2ccc(C(F)(F)F)cn2)cc(S(=O)(=O)NC(=O)COc2ccc3ccccc3c2)c1] 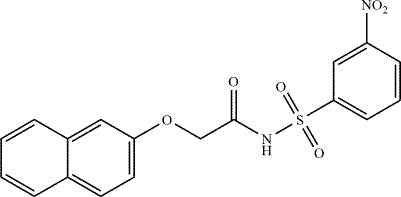 [SMILES: O=C(COc1ccc2ccccc2c1)NS(=O)(=O)c1cccc([N+](=O)[O-])c1] 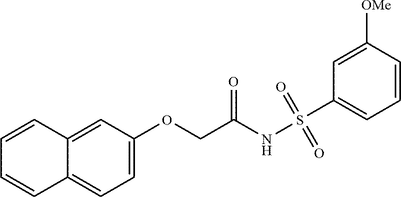 [SMILES: COc1cccc(S(=O)(=O)NC(=O)COc2ccc3ccccc3c2)c1] 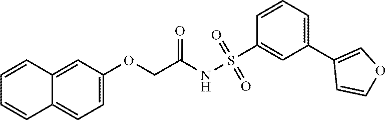 [SMILES: O=C(COc1ccc2ccccc2c1)NS(=O)(=O)c1cccc(-c2ccoc2)c1] 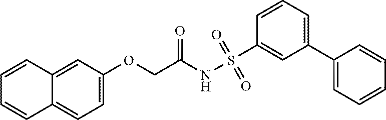 [SMILES: O=C(COc1ccc2ccccc2c1)NS(=O)(=O)c1cccc(-c2ccccc2)c1] 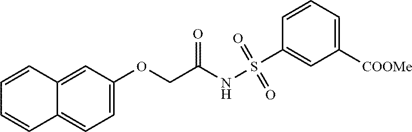 [SMILES: COC(=O)c1cccc(S(=O)(=O)NC(=O)COc2ccc3ccccc3c2)c1]
.

5. The compound according to claim 2, wherein R² and R³ are H, and R⁴ is 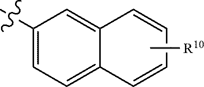 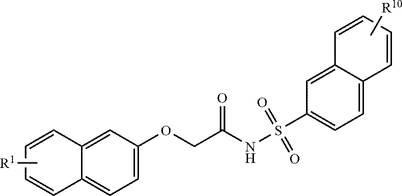
.

6. The compound according to claim 5, wherein the compound is: 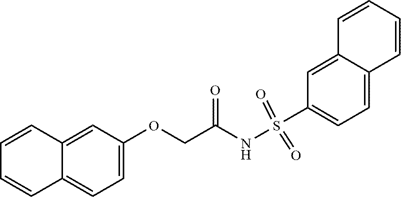 [SMILES: O=C(COc1ccc2ccccc2c1)NS(=O)(=O)c1ccc2ccccc2c1]
.

7. The compound according to claim 2, wherein R⁴ is: 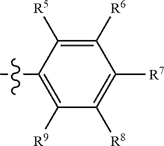
and R⁵ = R⁷ = R⁹ = alkyl.

8. The compound according to claim 7, wherein R⁵ = R⁷ = R⁹ = methyl.

9. The compound according to claim 8, wherein the compound is chosen from: 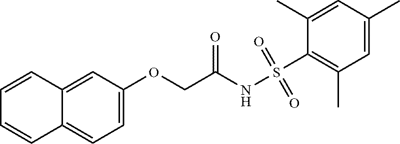 [SMILES: Cc1cc(C)c(S(=O)(=O)NC(=O)COc2ccc3ccccc3c2)c(C)c1] 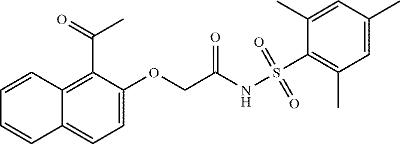 [SMILES: CC(=O)c1c(OCC(=O)NS(=O)(=O)c2c(C)cc(C)cc2C)ccc2ccccc12] 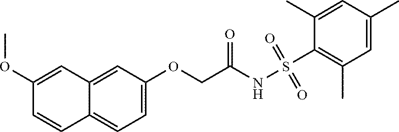 [SMILES: COc1ccc2ccc(OCC(=O)NS(=O)(=O)c3c(C)cc(C)cc3C)cc2c1] 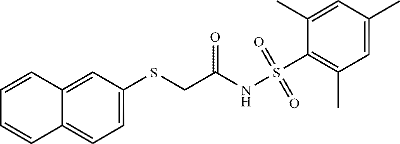 [SMILES: Cc1cc(C)c(S(=O)(=O)NC(=O)CSc2ccc3ccccc3c2)c(C)c1] 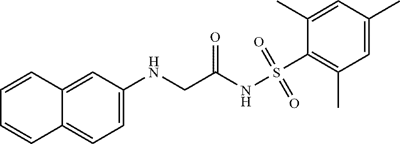 [SMILES: Cc1cc(C)c(S(=O)(=O)NC(=O)CNc2ccc3ccccc3c2)c(C)c1]
.

10. The compound according to claim 1, wherein the compound is chosen from: 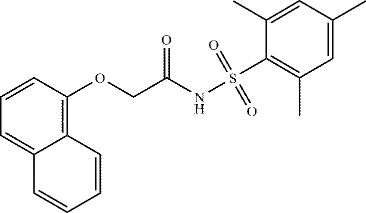 [SMILES: Cc1cc(C)c(S(=O)(=O)NC(=O)COc2cccc3ccccc23)c(C)c1] 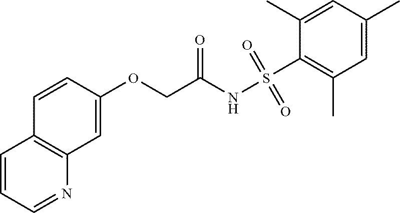 [SMILES: Cc1cc(C)c(S(=O)(=O)NC(=O)COc2ccc3cccnc3c2)c(C)c1] 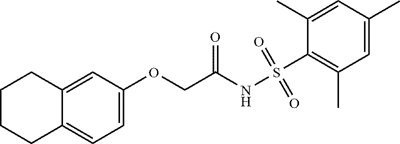 [SMILES: Cc1cc(C)c(S(=O)(=O)NC(=O)COc2ccc3c(c2)CCCC3)c(C)c1] 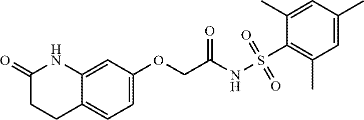 [SMILES: Cc1cc(C)c(S(=O)(=O)NC(=O)COc2ccc3c(c2)NC(=O)CC3)c(C)c1] 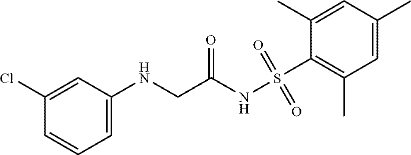 [SMILES: Cc1cc(C)c(S(=O)(=O)NC(=O)CNc2cccc(Cl)c2)c(C)c1] 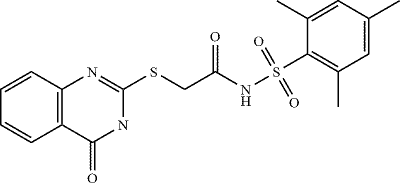 [SMILES: Cc1cc(C)c(S(=O)(=O)NC(=O)CSc2nc3ccccc3c(=O)[nH]2)c(C)c1] 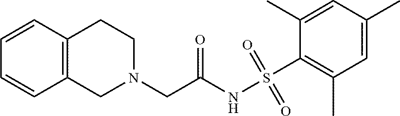 [SMILES: Cc1cc(C)c(S(=O)(=O)NC(=O)CN2CCc3ccccc3C2)c(C)c1] 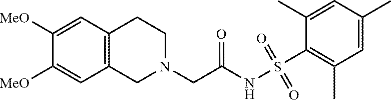 [SMILES: COc1cc2c(cc1OC)CN(CC(=O)NS(=O)(=O)c1c(C)cc(C)cc1C)CC2] 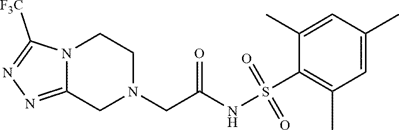 [SMILES: Cc1cc(C)c(S(=O)(=O)NC(=O)CN2CCn3c(nnc3C(F)(F)F)C2)c(C)c1] 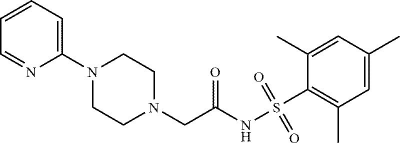 [SMILES: Cc1cc(C)c(S(=O)(=O)NC(=O)CN2CCN(c3ccccn3)CC2)c(C)c1] 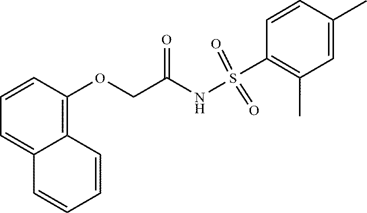 [SMILES: Cc1ccc(S(=O)(=O)NC(=O)COc2cccc3ccccc23)c(C)c1] 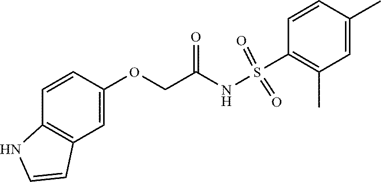 [SMILES: Cc1ccc(S(=O)(=O)NC(=O)COc2ccc3[nH]ccc3c2)c(C)c1] 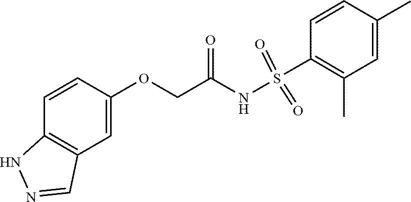 [SMILES: Cc1ccc(S(=O)(=O)NC(=O)COc2ccc3[nH]ncc3c2)c(C)c1]
.

11. A method of activating EPAC1 protein in cells, wherein said method comprises contacting one more cells with one or more compounds according to Formulas I, II, IIa, and/or IIb.

12. The method of claim 11, wherein said cells comprises a gene for expressing EPAC1 protein.

13. The method of claim 11, wherein said compound is a compound according to Formula IIa.

14. The method of claim 13, wherein said compound is chosen from: 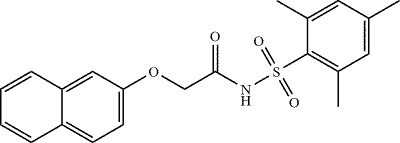 [SMILES: Cc1cc(C)c(S(=O)(=O)NC(=O)COc2ccc3ccccc3c2)c(C)c1] 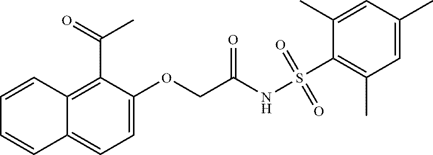 [SMILES: CC(=O)c1c(OCC(=O)NS(=O)(=O)c2c(C)cc(C)cc2C)ccc2ccccc12] 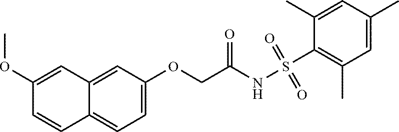 [SMILES: COc1ccc2ccc(OCC(=O)NS(=O)(=O)c3c(C)cc(C)cc3C)cc2c1] 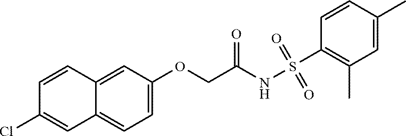 [SMILES: Cc1ccc(S(=O)(=O)NC(=O)COc2ccc3cc(Cl)ccc3c2)c(C)c1] 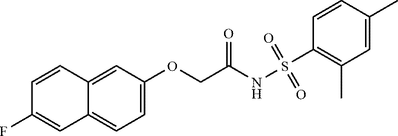 [SMILES: Cc1ccc(S(=O)(=O)NC(=O)COc2ccc3cc(F)ccc3c2)c(C)c1] 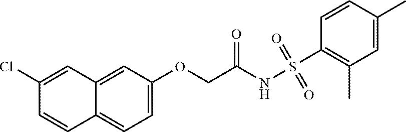 [SMILES: Cc1ccc(S(=O)(=O)NC(=O)COc2ccc3ccc(Cl)cc3c2)c(C)c1] 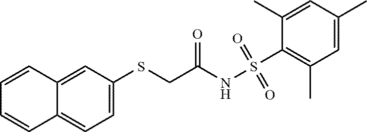 [SMILES: Cc1cc(C)c(S(=O)(=O)NC(=O)CSc2ccc3ccccc3c2)c(C)c1] 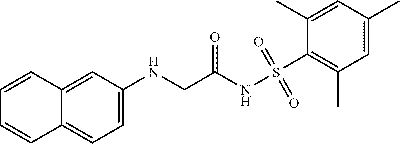 [SMILES: Cc1cc(C)c(S(=O)(=O)NC(=O)CNc2ccc3ccccc3c2)c(C)c1] 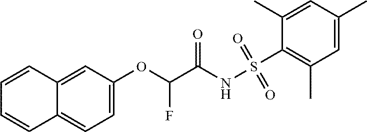 [SMILES: Cc1cc(C)c(S(=O)(=O)NC(=O)C(F)Oc2ccc3ccccc3c2)c(C)c1] 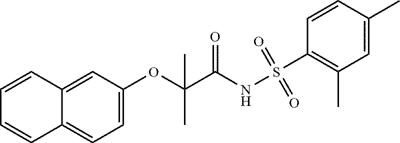 [SMILES: Cc1ccc(S(=O)(=O)NC(=O)C(C)(C)Oc2ccc3ccccc3c2)c(C)c1] 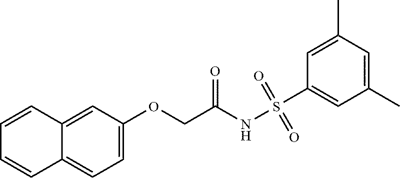 [SMILES: Cc1cc(C)cc(S(=O)(=O)NC(=O)COc2ccc3ccccc3c2)c1] 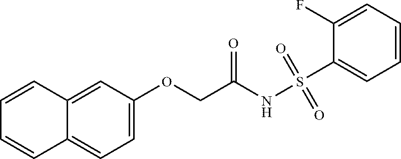 [SMILES: O=C(COc1ccc2ccccc2c1)NS(=O)(=O)c1ccccc1F] 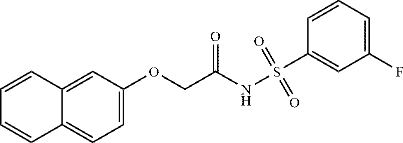 [SMILES: O=C(COc1ccc2ccccc2c1)NS(=O)(=O)c1cccc(F)c1] 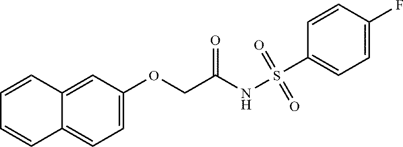 [SMILES: O=C(COc1ccc2ccccc2c1)NS(=O)(=O)c1ccc(F)cc1] 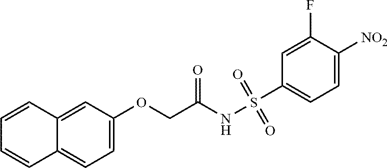 [SMILES: O=C(COc1ccc2ccccc2c1)NS(=O)(=O)c1ccc([N+](=O)[O-])c(F)c1] 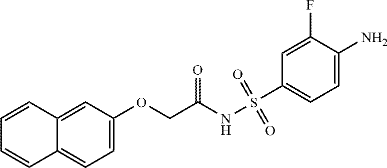 [SMILES: Nc1ccc(S(=O)(=O)NC(=O)COc2ccc3ccccc3c2)cc1F] 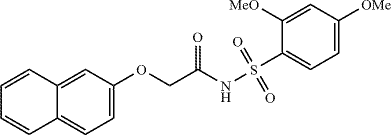 [SMILES: COc1ccc(S(=O)(=O)NC(=O)COc2ccc3ccccc3c2)c(OC)c1] 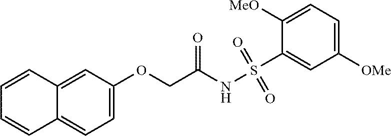 [SMILES: COc1ccc(OC)c(S(=O)(=O)NC(=O)COc2ccc3ccccc3c2)c1] 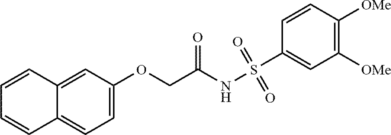 [SMILES: COc1ccc(S(=O)(=O)NC(=O)COc2ccc3ccccc3c2)cc1OC] 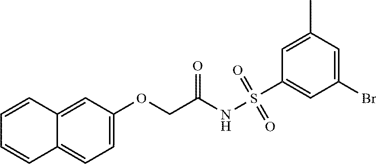 [SMILES: Cc1cc(Br)cc(S(=O)(=O)NC(=O)COc2ccc3ccccc3c2)c1] 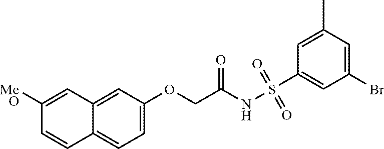 [SMILES: COc1ccc2ccc(OCC(=O)NS(=O)(=O)c3cc(C)cc(Br)c3)cc2c1] 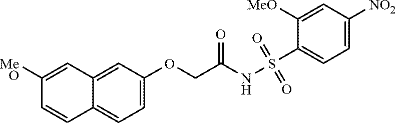 [SMILES: COc1ccc2ccc(OCC(=O)NS(=O)(=O)c3ccc([N+](=O)[O-])cc3OC)cc2c1] 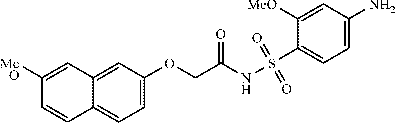 [SMILES: COc1ccc2ccc(OCC(=O)NS(=O)(=O)c3ccc(N)cc3OC)cc2c1] 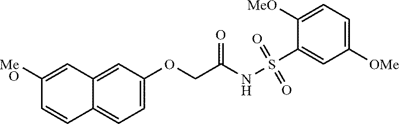 [SMILES: COc1ccc(OC)c(S(=O)(=O)NC(=O)COc2ccc3ccc(OC)cc3c2)c1] 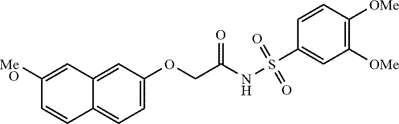 [SMILES: COc1ccc2ccc(OCC(=O)NS(=O)(=O)c3ccc(OC)c(OC)c3)cc2c1] 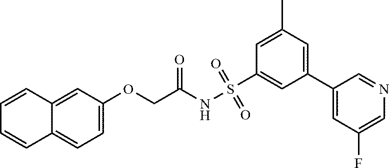 [SMILES: Cc1cc(-c2cncc(F)c2)cc(S(=O)(=O)NC(=O)COc2ccc3ccccc3c2)c1] 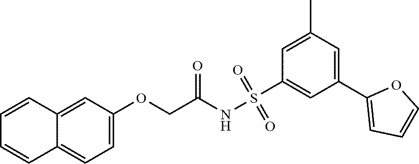 [SMILES: Cc1cc(-c2ccco2)cc(S(=O)(=O)NC(=O)COc2ccc3ccccc3c2)c1] 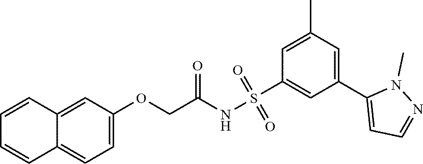 [SMILES: Cc1cc(-c2ccnn2C)cc(S(=O)(=O)NC(=O)COc2ccc3ccccc3c2)c1]
.

15. The method of claim 11, wherein said compound wherein said compound is a compound according to Formula IIb.

16. The method of claim 15, wherein the compound is: 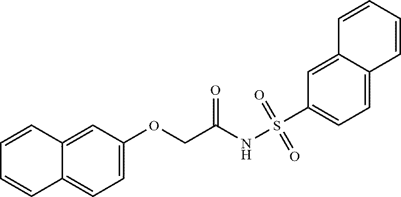 [SMILES: O=C(COc1ccc2ccccc2c1)NS(=O)(=O)c1ccc2ccccc2c1]
.
